(19) United States
(12) Patent Application Publication
(10) Pub. No.: US 2026/0335314 A1
(43) Pub. Date: Oct. 29, 2026

(54) CRYSTALLINE SALTS OR AMORPHOUS FORMS OF 2-((1H-PYRAZOL-3-YL)METHYL)-6-((6-AMINOPYRIDIN-2-YL)METHYL)-4-METHYL-4,6-DIHYDRO -5H-THIAZOLO[5',4':4,5]PYRROLO[2,3-D]PYRIDAZIN-5-ONE

(71) Applicant: Agios Pharmaceuticals, Inc., Cambridge, MA (US)

(72) Inventors: Daniel R. Fandrick, Littleton, MA (US); Louis Grenier, Sherbrooke (CA); Benjamin S. Lane, Lynnfield, MA (US); Jaemoon Lee, Andover, MA (US); Cheuk-Yui Leung, Acton, MA (US); Hui Li, Arlington, MA (US); Karlie E. Angelillo, Medford, MA (US); Eric Simone, West Newbury, MA (US); Jacob P. Sizemore, Dickinson, TX (US)

(21) Appl. No.: 19/484,160

(22) PCT Filed: May 15, 2024
(86) PCT No.: PCT/US2024/029471

§ 371 Date: Nov. 13, 2025

Related U.S. Application Data
(60) Provisional application No. 63/466,552, filed on May 15, 2023.

(51) Int. Cl. A61K 31/5025; A61K 9/20; C07C 59/255; C07D 513/14

(52) U.S. Cl. CPC A61K 31/5025; A61K 9/2027; A61K 9/2054; C07C 59/255; C07D 513/14

(57) ABSTRACT

Provided herein are crystalline salts and free base forms as well as amorphous forms of the compound having the formula:(I) Also provided are pharmaceutical compositions comprising such crystalline and amorphous forms, methods for their manufacture, and uses thereof for treating various conditions such as, hemolytic anemia, sickle cell disease and MDS (very low risk MDS, low risk MDS, lower risk MDS and/or intermediate risk MDS).

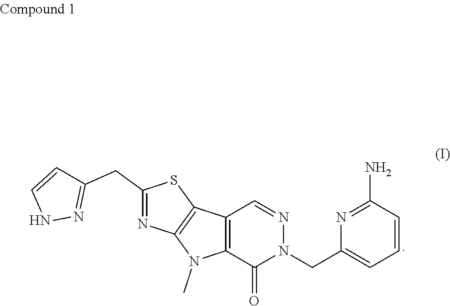 [SMILES: Cn1c2nc(Cc3cc[nH]n3)sc2c2cnn(Cc3cccc(N)n3)c(=O)c21]

### RELATED APPLICATIONS

This application claims priority to U.S. Provisional Application No. 63/466,552, filed May 15, 2023, the contents of which is incorporated herein in its entirety.

### BACKGROUND

Pyruvate kinase (PK) is a metabolic enzyme that converts phosphoenolpyruvate to pyruvate during glycolysis. Four PK isoforms exist in mammals: the L and R isoforms are expressed in liver and red blood cells, the M1 isoform is expressed in most adult tissues, and the M2 isoform is a splice variant of M1 expressed during embryonic development. A well-known difference between the M1 and M2 isoforms of PK is that M2 is a low-activity enzyme that relies on allosteric activation by the upstream glycolytic intermediate, fructose-1,6-bisphosphate (FBP), whereas M1 is a constitutively active enzyme. PK activators can be used to treat a number of different disorders including PKD (Pyruvate Kinase Deficiency), thalassemia (e.g., alpha and beta-thalassemia), hereditary elliptocytosis, abetalipoproteinemia or Bassen-Kornzweig syndrome, sickle cell disease, paroxysmal nocturnal hemoglobinuria, and various anemias which include congenital anemias (e.g., enzymopathies) and various hemolytic anemias, (e.g. hereditary and/or congenital hemolytic anemia, acquired hemolytic anemia, chronic hemolytic anemia caused by phosphoglycerate kinase deficiency, anemia due to MDS (myelodysplastic syndromes) including very low risk, low risk, lower risk and/or intermediate risk MDS, non-spherocytic hemolytic anemia and hereditary spherocytosis).

2-((1H-pyrazol-3-yl)methyl)-6-((6-aminopyridin-2-yl)methyl)-4-methyl-4,6-dihydro-5H-thiazolo[5′,4′:4,5]pyrrolo[2,3-d]pyridazin-5-one, herein referred to as Compound 1, is useful for activating pyruvate kinase in a subject in need thereof. See e.g., WO 2019/035865 and WO 2019/035864, the contents of which are incorporated herein by reference.

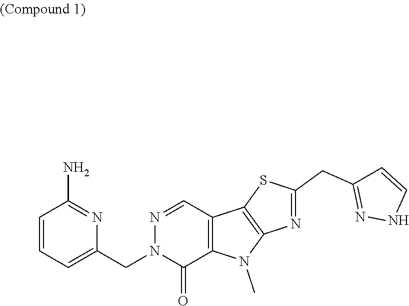 [SMILES: Cn1c2nc(Cc3cc[nH]n3)sc2c2cnn(Cc3cccc(N)n3)c(=O)c21]

Compound 1 is currently being investigated in clinical trials for use in adult patients with sickle cell disease as well as in patients with anemia due to MDS (lower risk to intermediate risk MDS). See e.g., U.S. clinical trials identifiers NCT04536792 and NCT05490446.

### SUMMARY

Provided herein are crystalline free base forms of Compound 1.

Also provided herein are crystalline salt forms of Compound 1.

In one embodiment, the crystalline salt form described herein is a hemi-hydrate hemi-sulfate salt of Compound 1. In some aspects the hemi-hydrate hemi-sulfate salt of Compound 1 is represented as

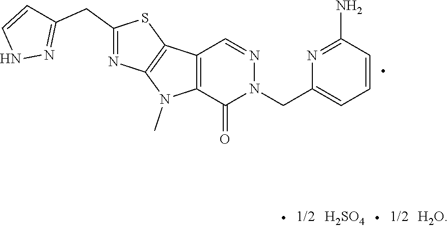 [SMILES: Cn1c2nc(Cc3cc[nH]n3)sc2c2cnn(Cc3cccc(N)n3)c(=O)c21 ; "H2SO4" ; "1/2" ; "H2O."]

In one embodiment, the crystalline salt form described herein is a phosphate salt of Compound 1. In some aspects the phosphate salt of Compound 1 is represented as

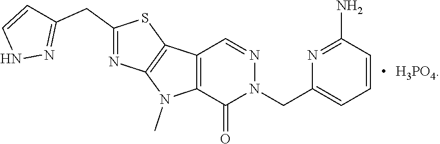

In one embodiment, the crystalline salt form described herein is a DL-tartrate salt of Compound 1. In some aspects the DL-tartrate salt of Compound 1 is represented as

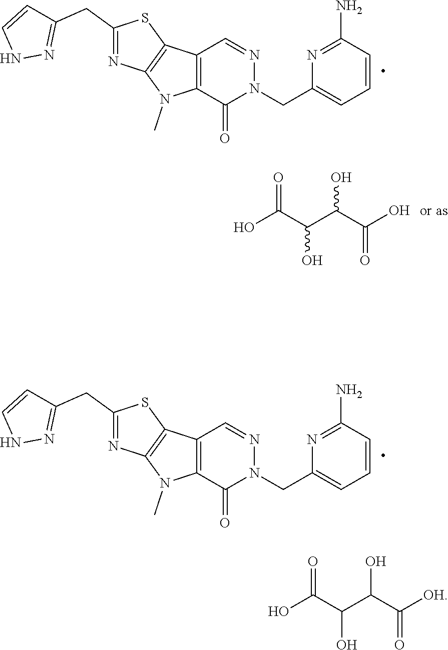

In one embodiment, the crystalline salt form described herein is an L-tartrate salt of Compound 1. In some aspects the L-tartrate salt of Compound 1 is represented as

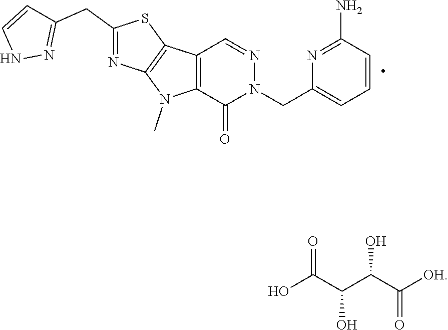 [SMILES: Cn1c2nc(Cc3cc[nH]n3)sc2c2cnn(Cc3cccc(N)n3)c(=O)c21 ; O=C(O)[C@@H](O)[C@H](O)C(=O)O]

In one embodiment, the crystalline salt form described herein is a hydrochloride salt of Compound 1. In some aspects the hydrochloride salt of Compound 1 is represented as

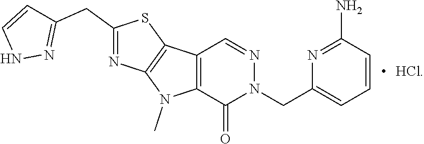

In one embodiment, the crystalline form described herein is a free base monohydrate form of Compound 1. In some aspects the free base monohydrate of Compound 1 is represented as

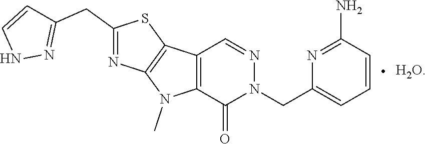

Also provided herein are pharmaceutical compositions comprising one or more of the described crystalline forms or an amorphous form of Compound 1. Methods for the preparation of such forms and uses thereof, including their use for treating conditions such as, e.g., hemolytic anemia, sickle cell disease, and anemia due to MDS (very low risk MDS, low risk MDS, lower risk MDS and/or intermediate risk MDS) are also described.

### BRIEF DESCRIPTION OF THE FIGURES

FIG. 1 depicts an X-ray powder diffraction pattern (XRPD) for crystalline hemi-hydrate hemi-sulfate salt Form A of Compound 1.

FIG. 2 depicts the combined thermogravimetric analysis (TGA) thermogram and differential scanning calorimetry (DSC) thermogram for crystalline hemi-hydrate hemi-sulfate salt Form A of Compound 1.

FIG. 3 depicts the dynamic vapor sorption (DVS) isotherm for crystalline hemi-hydrate hemi-sulfate salt Form A of Compound 1.

FIG. 4 depicts an X-ray powder diffraction pattern for crystalline phosphate salt Form B of Compound 1.

FIG. 5 depicts the combined thermogravimetric analysis (TGA) thermogram and differential scanning calorimetry (DSC) thermogram for crystalline phosphate salt Form B of Compound 1

FIG. 6 depicts the dynamic vapor sorption (DVS) isotherm for crystalline phosphate acid salt Form B.

FIG. 7 depicts an X-ray powder diffraction pattern for crystalline DL-tartrate salt Form C of Compound 1.

FIG. 8 depicts the combined thermogravimetric analysis (TGA) thermogram and differential scanning calorimetry (DSC) thermogram for crystalline DL-tartrate salt Form C of Compound 1.

FIG. 9 depicts the dynamic vapor sorption (DVS) isotherm for crystalline DL-tartrate salt Form C of Compound 1.

FIG. 10 depicts an X-ray powder diffraction pattern for crystalline hydrochloride salt Form D of Compound 1.

FIG. 11 depicts the combined thermogravimetric analysis (TGA) thermogram and differential scanning calorimetry (DSC) thermogram for crystalline hydrochloride salt Form D of Compound 1.

FIG. 12 depicts an X-ray powder diffraction pattern for crystalline free base monohydrate Form E of Compound 1.

FIG. 13 depicts the combined thermogravimetric analysis (TGA) thermogram and differential scanning calorimetry (DSC) thermogram for crystalline free base monohydrate Form E of Compound 1.

FIG. 14 depicts the dynamic vapor sorption (DVS) isotherm for crystalline free base monohydrate Form E of Compound 1.

FIG. 15 depicts a graph showing the pH solubility plot of Forms B and C of Compound 1 in pH of 2, 4.5, and 6.8.

FIG. 16 depicts an overlay of X-ray powder diffraction patterns of solid residue of Form B of Compound 1 taken at each timestamp in Table 1.

FIG. 17 depicts an overlay of X-ray powder diffraction patterns of solid residue of Form C of Compound 1 taken at each timestamp in Table 3.

FIG. 18 depicts a graph showing the solubility in bio relevant media plot of Form B and C of Compound 1.

FIG. 19 depicts an overlay of X-ray powder diffraction patterns of solid residue of Form B of Compound 1 taken at each timestamp in Table 4.

FIG. 20 depicts an overlay of X-ray powder diffraction patterns of solid residue of Form C of Compound 1 taken at each timestamp in Table 5.

FIG. 21A depicts mean plasma concentration-time profiles of Compound 1 after a PO (per os) dose of free base monohydrate Form E.

FIG. 21B depicts mean plasma concentration-time profiles of Compound 1 after a PO dose of free base monohydrate Form E (unmilled).

FIG. 22A depicts mean plasma concentration-time profiles of Compound 1 after a PO dose of Form B.

FIG. 22B depicts mean plasma concentration-time profiles of Compound 1 after a PO dose of Form C.

FIG. 23A depicts mean plasma concentration-time profiles of Compound 1 after a PO dose of Form A.

FIG. 23B depicts mean plasma concentration-time profiles of Compound 1 after a PO dose of a formulation (SDD with PVPVA and 35% loading of Compound 1) of Compound 1.

FIG. 24A depicts mean plasma concentration-time profiles of Compound 1 after a PO dose of a formulation (SDD with HPMC-AS and 35% loading of Compound 1) of Compound 1.

FIG. 24B depicts a graph showing the aqueous solubility of Forms A, B, C, and E.

FIG. 25A depicts the solubility of Forms A, B, C, and E.

FIG. 25B depicts the stability of two formulations comprising amorphous Compound 1.

FIG. 26 depicts an X-ray powder diffraction pattern for a formulation comprising amorphous Compound 1.

FIG. 27 depicts an X-ray powder diffraction pattern (XRPD) for crystalline hydrochloride salt Form D-1 of Compound 1.

FIG. 28 depicts the thermogravimetric analysis (TGA) thermogram for crystalline hydrochloride salt Form D-1 of Compound 1.

FIG. 29 depicts the differential scanning calorimetry (DSC) thermogram for crystalline hydrochloride salt Form D-1 of Compound 1.

FIG. 30 depicts the dynamic vapor sorption (DVS) isotherm for crystalline hydrochloride salt Form D-1 of Compound 1.

FIG. 31 depicts an X-ray powder diffraction pattern (XRPD) for crystalline hydrochloride salt Form D-2 of Compound 1.

FIG. 32 depicts the thermogravimetric analysis (TGA) thermogram for crystalline hydrochloride salt Form D-2 of Compound 1.

FIG. 33 depicts the differential scanning calorimetry (DSC) thermogram for crystalline hydrochloride salt Form D-2 of Compound 1.

FIG. 34 depicts the dynamic vapor sorption (DVS) isotherm for crystalline hydrochloride salt Form D-2 of Compound 1.

FIG. 35 depicts an X-ray powder diffraction pattern (XRPD) for crystalline L-tartrate salt Form I of Compound 1.

FIG. 36 depicts the thermogravimetric analysis (TGA) thermogram for crystalline L-tartrate salt Form I of Compound 1.

FIG. 37 depicts the differential scanning calorimetry (DSC) thermogram for crystalline L-tartrate salt Form I of Compound 1.

FIG. 38 depicts the dynamic vapor sorption (DVS) isotherm for crystalline L-tartrate salt Form I of Compound 1.

FIG. 39 depicts an X-ray powder diffraction pattern (XRPD) for crystalline hemi-hydrate hemi-sulfate salt Form H of Compound 1.

FIG. 40 depicts the differential scanning calorimetry (DSC) thermogram for crystalline hemi-hydrate hemi-sulfate salt Form H of Compound 1.

FIG. 41 depicts the thermogravimetric analysis (TGA) thermogram for crystalline hemi-hydrate hemi-sulfate salt Form L of Compound 1.

FIG. 42 depicts an X-ray powder diffraction pattern (XRPD) for crystalline DL-tartrate salt Form F of Compound 1.

FIG. 43 depicts the combined thermogravimetric analysis (TGA) thermogram and differential scanning calorimetry (DSC) thermogram for crystalline DL-tartrate salt Form F of Compound 1.

FIG. 44 depicts an X-ray powder diffraction pattern (XRPD) for crystalline DL-tartrate salt Form G of Compound 1.

FIG. 45 depicts the combined thermogravimetric analysis (TGA) thermogram and differential scanning calorimetry (DSC) thermogram for crystalline DL-tartrate salt Form G of Compound 1.

FIG. 46 depicts an X-ray powder diffraction pattern (XRPD) for crystalline hydrochloride salt Form J of Compound 1.

FIG. 47 depicts the combined thermogravimetric analysis (TGA) thermogram and differential scanning calorimetry (DSC) thermogram for crystalline hydrochloride salt Form J of Compound 1.

FIG. 48 depicts an X-ray powder diffraction pattern (XRPD) for crystalline hydrochloride salt Form K of Compound 1.

FIG. 49 depicts the combined thermogravimetric analysis (TGA) thermogram and differential scanning calorimetry (DSC) thermogram for crystalline hydrochloride salt Form K of Compound 1.

FIG. 50 depicts an X-ray powder diffraction pattern for crystalline DL-tartrate salt Form C-1 of Compound 1.

FIG. 51 depicts the differential scanning calorimetry (DSC) thermogram for crystalline DL-tartrate salt Form C-1 of Compound 1.

FIG. 52 depicts the thermogravimetric analysis (TGA) thermogram for crystalline DL-tartrate salt Form C-1 of Compound 1.

FIG. 53 depicts the dynamic vapor sorption (DVS) isotherm for crystalline DL-tartrate salt Form C-1 of Compound 1.

### DETAILED DESCRIPTION

Provided herein are various crystalline free-base and crystalline salt forms of Compound 1 and methods for preparing and using such crystalline free-base and crystalline salt forms of Compound 1. Also provided herein is an amorphous form of Compound 1 and the methods for preparing an amorphous form of Compound 1. Also provided herein are pharmaceutical formulations comprising crystalline and/or amorphous forms of Compound 1 and methods for preparing and using such formulations comprising crystalline and/or amorphous forms of Compound 1.

### Definitions

As used herein, the recitation of a range of values is intended to serve as a shorthand method of referring individually to each separate value falling within the range as well as the highest and lowest values that define the range and that each value is incorporated into the specification as if it were individually recited herein, unless expressly stated to the contrary. For example, a range of values from X to Y includes both X and Y and all the values in between X and Y.

The use of any and all examples, or exemplary language (e.g., “such as” and “e.g.” and “i.e.”) provided herein, is intended to better illustrate the disclosure and is not a limitation on the scope of the disclosure unless otherwise claimed. Phrases such as “in one aspect” or “in one embodiment” should not be construed as indicating that such elements occur or exist in isolation or that such elements are not shared by other aspects or embodiments of the disclosure. Rather, it should be understood that all aspects and embodiments may be freely combined with any and all other aspects and embodiments of the disclosure as described herein. No language in the specification should be construed as indicating that any non-claimed element is essential to the practice of the disclosure.

When used alone, the terms “Form A”, “Form B”, “Form C”, “Form D”, and “Form E refer to the crystalline forms A, B, C, D, and E of Compound 1, respectively. Alternatively, when used alone, the terms “Form F”, “Form G”, “Form H”, “Form I”, “Form J”, and “Form K” refer to the crystalline forms F, G, H, I, J, and K. The terms “Form A”, “crystalline Form A”, and “crystalline hemi-hydrate hemi-sulfate salt Form A of Compound 1” are used interchangeably. Similarly, “Form B”, “crystalline Form B”, and “crystalline phosphate salt Form B of Compound 1” are used interchangeably. Similarly, “Form C”, “crystalline Form C”, and “crystalline DL-tartrate salt Form C of Compound 1” are used interchangeably. Additional examples of crystalline DL-tartrate salt Form C include crystalline DL-tartrate Forms C-1 and C-2. Without being bound by theory, Form C-1 may be the same or substantially the same crystalline form as Form C. More specifically, Form C-1 may have different water content levels, yet still have the same or substantially the same XRPD as well as other characterization data, including DSC data, as Form C. Similarly, “Form D”, “crystalline Form D”, and “crystalline hydrochloride salt Form D of Compound 1” are used interchangeably. Additional examples of crystalline hydrochloride salt Form D include crystalline hydrochloride Forms D-1 and D-2. Without being bound by theory, Forms D-1 and D-2 may be the same or substantially the same crystalline form as Form D. More specifically, Forms D-1 and D-2 may have different water content levels, yet still have the same or substantially the same XRPD as well as other characterization data, including DSC data, as Form D. Similarly, “Form E”, “crystalline Form E”, and “crystalline free base monohydrate Form E of Compound 1” are used interchangeably. Similarly, “Form F”, “crystalline Form F”, and “crystalline DL-tartrate salt Form F of Compound 1” are used interchangeably. Similarly, “Form G”, “crystalline Form G”, and “crystalline DL-tartrate salt Form G of Compound 1” are used interchangeably. Similarly, “Form I”, “crystalline Form I”, and “crystalline L-tartrate salt Form I of Compound 1” are used interchangeably. Similarly, “Form J”, “crystalline Form J”, and “crystalline hydrochloride salt Form K of Compound 1” are used interchangeably. Similarly, “Form K”, “crystalline Form K”, and “crystalline hydrochloride salt Form K of Compound 1” are used interchangeably. Similarly, the terms “Form H”, “crystalline Form H”, and “crystalline hemi-hydrate hemi-sulfate salt Form H of Compound 1” are used interchangeably.

“Pattern A”, “Pattern B”, “Pattern C”, “Pattern D”, and “Pattern E” refer to the X-ray powder diffraction pattern (XRPD) for crystalline Form A, Form B, Form C, Form D, and Form E respectively. Alternatively, “Pattern C”, “Pattern D”, “Pattern F”, “Pattern G”, “Pattern H”, “Pattern I”, “Pattern J”, and “Pattern K” refer to the X-ray powder diffraction pattern (XRPD) for crystalline Form C-1, Form D-1, Form D-2, Form F, Form G, Form H, Form I, Form J, and Form K, respectively.

As used herein, the terms “crystalline free base,” “free-base crystalline form of Compound 1,” “crystalline free base form of Compound 1,” and “crystalline free base of Compound 1” are used interchangeably and mean the monohydrate free base (i.e., monohydrate non-salt) Form E of Compound 1, which is present in a crystalline form as Form E, unless expressly stated to the contrary.

Unless defined otherwise, the crystalline forms described herein are present as a single crystal or a plurality of crystals in which each crystal in the plurality is the same crystal form, i.e., a single crystalline form in which no other detectable amounts of other crystalline forms are present. In some aspects, however, where a crystal form is defined as a specified percentage of one particular single crystalline form of the compound, the remainder is made up of amorphous form and/or crystalline forms other than the one or more particular forms that are specified. In some embodiments this is referred to as “phase purity”. As used herein, the term “phase purity” refers to a determination of the number of crystalline phases of the same material in a sample or composition and can be expressed as a percent by weight of the material or sample. In such aspects, the recited crystalline forms may comprise at least 60% of a single crystalline form, at least 70% of a single crystalline form, at least 80% of a single crystalline form, at least 90% of a single crystalline form, at least 95% of a single crystalline form, or at least 99% of a single crystalline form by weight. Accordingly, for example, if a material is described as comprising at least 60% of a single crystalline form, that material may be described as being at least 60% phase pure or as having a phase purity of at least 60%. Percent by weight of a particular crystal form is determined by the weight of the particular crystalline form divided by the sum weight of the particular crystal, plus the weight of the other crystal forms present plus the weight of amorphous form present multiplied by 100%.

As used herein, the term “anhydrous” means that the referenced crystalline form has substantially no water in the crystal lattice e.g., less than about 0.1% by weight as determined by Karl Fisher analysis. In some embodiments, the crystalline forms as described herein may have a certain level of hydration or a particular water content but may or may not be a true hydrate of the particular crystalline salt.

As used herein, the term “amorphous” means a solid material that is present in a non-crystalline state or form. Amorphous solids are disordered arrangements of molecules and therefore possess no distinguishable crystal lattice or unit cell and consequently have no definable long-range ordering. Solid state ordering of solids may be determined by standard techniques known in the art, e.g., by X-ray powder diffraction (XRPD) or differential scanning calorimetry (DSC). Amorphous solids can also be differentiated from crystalline solids e.g., by birefringence using polarized light microscopy.

As used herein, “solid state” or “solid state form” refers to a compound, composition, formulation, or solid state dispersion that is an amorphous solid or an crystalline solid. In some embodiments, the solid state form is an amorphous (i.e., non-crystalline) form. In some embodiments, the solid state form is a crystalline form (e.g., a polymorph, salt, free base, solvate, or hydrate).

As used herein, chemical purity refers to the extent by which the disclosed crystalline or amorphous form(s) is free from other materials having a structural formula that is different from the structural formula of the crystalline salts, crystalline free base forms or amorphous forms of Compound 1 disclosed herein and are referred to as impurities. The chemical purity of the disclosed crystalline (or amorphous) form(s) is represented as the weight of the crystalline (or amorphous) form divided by the sum of the weight of the crystalline (or amorphous) form plus the weight of the impurities, multiplied by 100%, i.e., percent by weight. In various embodiments, a disclosed crystalline or amorphous form(s) has a chemical purity of at least 60%, at least 70%, at least 80%, at least 90%, at least 95%, or at least 99% by weight, as measured by HPLC.

As used herein, “crystalline” refers to a solid form of a compound wherein there exists long-range atomic order in the positions of the atoms. The crystalline nature of a solid can be confirmed, for example, by examination of the X-ray powder diffraction pattern. If the XRPD of a particular solid compound shows peaks (as opposed to broad, substantially featureless humps that would be expected for an amorphous or non-crystalline material) in the XRPD spectra (or pattern), then the compound is crystalline.

As used herein, the term “solvate” refers to a crystalline compound having a stoichiometric or non-stoichiometric amount of solvent, or mixture of solvents, incorporated into its crystal structure or crystal lattice. As used herein, the term “unsolvated” refers to a crystalline compound that has substantially no solvent molecules incorporated into its crystal structure or crystal lattice.

The term “hydrate” refers to a crystalline compound having a stoichiometric or non-stoichiometric amount of water incorporated into or associated with its crystal structure or crystal lattice. A hydrate is a solvate wherein the solvent incorporated into or associated with the crystal structure or crystal lattice is water.

As used herein, “2-((1H-pyrazol-3-yl)methyl)-6-((6-aminopyridin-2-yl)methyl)-4-methyl-4,6-dihydro-5H-thiazolo[5′,4′:4,5]pyrrolo[2,3-d]pyridazin-5-one”, “Compound 1”, and “free base of Compound 1” and “free base (non-salt) of Compound 1” are used interchangeably and refer to the compound having the following structural formula:

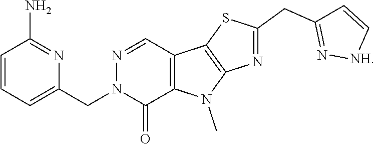 [SMILES: Cn1c2nc(Cc3cc[nH]n3)sc2c2cnn(Cc3cccc(N)n3)c(=O)c21]

As used herein, the term “hemi-hydrate hemi-sulfate” means the stoichiometric ratio of Compound 1 to H₂SO₄ and H₂O is 2:1:1 in a crystalline form (i.e. a crystalline form contains two molecules of Compound 1 per one molecule of H₂SO₄ and H₂O). The hemi-hydrate hemi-sulfate is also depicted herein as 1:0.5:0.5 or as

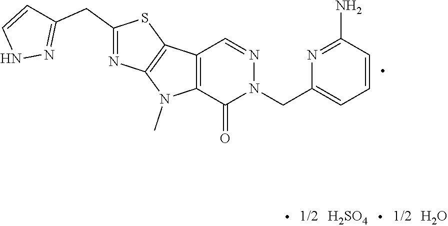 [SMILES: Cn1c2nc(Cc3cc[nH]n3)sc2c2cnn(Cc3cccc(N)n3)c(=O)c21 ; "H2SO4" ; "1/2" ; O]

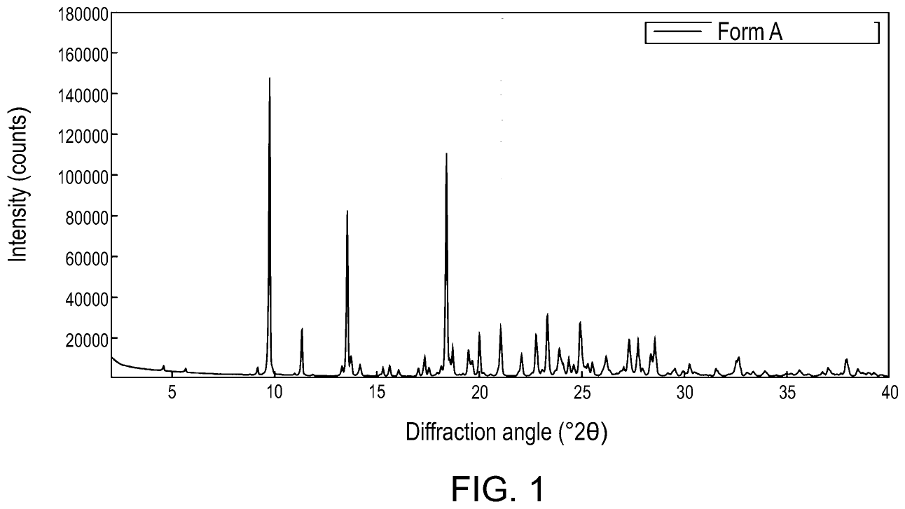

As used herein, the terms “substantially the same XRPD pattern” or “an X-ray powder diffraction pattern substantially similar to” when used with reference to a specific figure included in this disclosure (i.e., FIGS. 1, 4, 7, 10, 12, 26, 27, 31, 35, 39, 42, 44, 46, and/or 48) mean that for comparison purposes, at least 90% of the peaks shown in the XRPD of the specified figure (i.e., FIGS. 1, 4, 7, 10, 12, 26, 27, 31, 35, 39, 42, 44, 46, and/or 48) are present. It should be understood that unless expressly stated to the contrary the intended comparison is between a sample of isolated, substantially chemically pure, crystalline material (i.e., drug substance or API) and a particular crystalline form or Figure from the present disclosure and not a sample where the crystalline material is present together or mixed with other crystalline materials such as crystalline excipients, i.e., drug product, or other crystalline impurities (such as a mixture of different crystalline forms of the same compound).

One skilled in the art will understand that an XRPD pattern or diffractogram may be obtained which has one or more measurement errors depending on the recording conditions, such as the equipment or machine used. Similarly, it is generally known that intensities in an XRPD pattern may fluctuate depending on measurement conditions or sample preparation as a result of preferred orientation or crystal quality or size. Persons skilled in the art of XRPD will further realize that the relative intensity of peaks can also be affected by, for example, particles above 30 m in size and non-unitary aspect ratios. The skilled person understands that the position of reflections can be affected by the precise height at which the sample sits in the diffractometer, and also the zero calibration of the diffractometer. The surface planarity of the sample may also have a small effect on the XRPD pattern or diffractogram.

As a result of these considerations, the diffraction pattern data presented are not to be taken as absolute values (Jenkins, R & Snyder, R. L. ‘Introduction to X-Ray Powder Diffractometry’ John Wiley & Sons 1996; Bunn, C. W. (1948), ‘Chemical Crystallography’, Clarendon Press, London; Klug, H. P. & Alexander, L. E. (1974), ‘X-Ray Diffraction Procedures’). It should also be understood that the solid forms embodied herein are not limited to those that provide XRPD patterns that are identical to the XRPD pattern shown in the Figures, and any solid forms providing XRPD patterns substantially the same as those shown in the Figures fall within the scope of the corresponding embodiment and claims. A person skilled in the art of XRPD is able to judge the substantial identity of XRPD patterns. Generally, a measurement error of a diffraction angle in an XRPD is approximately 2θ (±0.2°), and such degree of a measurement error for each “peak” in the diffractogram pattern should be taken into account when considering the X-ray powder diffraction. It should be further understood that the 2-theta values provided herein were obtained using Cu Kal radiation.

A person skilled in the art also understands that the value or range of values observed in a particular compound's DSC thermogram will show variation between batches of different purities. Therefore, whilst for one compound the range may be small, for others the range may be quite large. Generally, a measurement error in DSC thermal events is approximately plus or minus 5° C., and such degree of a measurement error should be taken into account when considering the DSC data included herein. TGA thermograms show similar variations, such that a person skilled in the art recognizes that such measurement errors should be taken into account when judging the substantial identity between TGA thermograms.

Certain values provided herein may be rounded to avoid reporting insignificant figures. For example, the X-ray diffraction two theta values may be rounded to the tenths. One of skill in the art would readily understand the use of rounding in significant figures. With respect to the number “5” or greater in the hundredth position, the number in the tenth position is rounded up. However, if a value has the number “4” or less in the hundredth position, the number in the tenth position is not changed.

As used herein, the terms “effective amount” or “therapeutically effective amount” are used interchangeably and refer to an amount of a crystalline or amorphous form of Compound 1 described herein that is sufficient to provide a therapeutic benefit in the treatment of a condition or to delay the onset of or to minimize or reduce one or more symptoms associated with the condition. A therapeutically effective amount of a described crystalline or amorphous form of Compound 1 means an amount of the crystalline or amorphous form, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In some embodiments an effective amount of a crystalline or amorphous form of Compound 1 is between about 0.01 mg to about 100 mg that is administered once or twice daily. In some embodiments an effective amount of a crystalline salt of Compound 1 is an amount that is equivalent to about 0.01 mg to about 100 mg of the non-hydrated non-salt (free base) of Compound 1 that is administered once or twice daily. In some embodiments an effective amount of a crystalline salt form of Compound 1 that is a hydrate or solvate is an amount that is equivalent to about 0.01 mg to about 100 mg of the non-hydrated non-salt (free base) of Compound 1 that is administered once or twice daily. In some aspects, an effective amount of any of the crystalline or amorphous forms of Compound 1 described herein is an amount that is equivalent to about 2 mg, about 5 mg, about 7 mg, about 10 mg, about 15 mg, about 20 mg, about 25 mg, or about 30 mg of the non-hydrated non-salt (free base) of Compound 1 that is administered once daily.

In certain embodiments, a therapeutically effective amount is an amount sufficient for regulating 2,3-diphosphoglycerate and/or ATP levels in the blood of a patient in need thereof. In other embodiments, a therapeutically effective amount is an amount sufficient for treating hemolytic anemia. In other embodiments, a therapeutically effective amount is an amount sufficient for treating sickle cell disease or acquired hemolytic anemia including anemia due to MDS (including very low risk MDS, low risk MDS, lower risk MDS and/or intermediate risk MDS). In other embodiments, a therapeutically effective amount is an amount sufficient for treating acquired PKD. In other embodiments, a therapeutically effective amount is an amount sufficient for treating PKD that is acquired as a result of having another disease such as MDS (e.g. acquired PKD), including very low risk MDS, low risk MDS, lower risk MDS and/or intermediate risk MDS.

In one aspect, the MDS described herein is low risk MDS as characterized by the Revised International Prognostic Scoring System (IPSS-R) for MDS. See, for example, Greenberg PL, Tuechler H, Schanz J, et al. Revised International Prognostic Scoring System for myelodysplastic syndromes. Blood. 2012; 120:2454-2465. Low risk MDS includes, for example, an IPSS-R score of greater than 1.5 to 3. Alternatively, the MDS described herein is very low risk MDS as characterized by the Revised International Prognostic Scoring System (IPSS-R) for MDS. Very low risk MDS includes, for example, an IPSS-R score of less than or equal to 1.5. In another alternative, the MDS described herein is intermediate risk MDS as characterized by the Revised International Prognostic Scoring System (IPSS-R) for MDS. Intermediate risk MDS includes, for example, an IPSS-R score of greater than 3 to 4.5. In still another alternative, the term “lower risk MDS” used herein to describe MDS encompasses very low risk MDS and low risk MDS as described above.

In certain aspects, a therapeutically effective amount of a crystalline or amorphous form of Compound 1 is the amount required to generate a hemoglobin (Hb) response in a patient in need thereof is an increase of about ≥1.0 g/dL, ≥1.5 g/dL or ≥2.0 g/dL in Hb concentration from a baseline Hb concentration. In certain embodiments a patient in need thereof is a patient that has been diagnosed with hemolytic anemia. In other embodiments, a patient in need thereof has been diagnosed with sickle cell disease or acquired hemolytic anemia including anemia due to MDS. In still other embodiments a patient in need thereof is a patient that has been diagnosed with PKD that has been acquired as a result of the patient having MDS, including very low risk MDS, low risk MDS, lower risk MDS and/or intermediate risk MDS.

In certain aspects, a therapeutically effective amount of a crystalline or amorphous form of Compound 1 is the amount required to reduce a patient's transfusion burden over a period of time. As used herein, the term “reduction in transfusion burden” means at least a 20% reduction in the number of RBC (red blood cell) units transfused within at least 5 weeks of treatment. In certain embodiments, a reduction in transfusion burden is about a ≥33% reduction in the number of RBC units transfused within at least 5 weeks of treatment. In certain embodiments, a reduction of transfusion burden is about a ≥33% reduction in the number of RBC units transfused within at least 10 weeks, at least 20 weeks or at least 24 weeks of treatment.

As used herein, the term “hemolytic anemia” refers to a sub-type of anemia where a subject's low red blood cell count is caused by the destruction rather than the underproduction of red blood cells. Unless stated otherwise the term “anemia” as used herein refers to a low red blood cell count that is caused by underproduction of red blood cells, including ineffective erythropoiesis.

As used herein, the terms “MDS-associated anemia”, “anemia associated with MDS”, “anemia due to MDS”, “acquired hemolytic anemia associated with MDS” and “acquired anemia associated with MDS” are used interchangeably and refer to anemia that has developed or has been acquired in a subject as a result of having or suffering from MDS (very low risk MDS, low risk MDS, lower risk MDS and/or intermediate risk MDS).

As used herein, the terms “anemia associated with acquired PK deficiency (PKD)” and “PKD-associated anemia” or “acquired PK deficiency” and “acquired PKD” are used interchangeably and refer to anemia related to pyruvate kinase deficiency (PKD) which has developed in a subject that has or is suffering from MDS (very low risk MDS, low risk MDS, lower risk MDS and/or intermediate risk MDS). In some embodiments the anemia associated with acquired PK deficiency in a subject suffering from MDS (very low MDS, low risk MDS, lower risk MDS and/or intermediate risk MDS) is hemolytic anemia.

As used herein the terms “treatment,” “treat,” and “treating” refer to reversing, alleviating, delaying the onset of, reducing the likelihood of developing, or inhibiting the progress of a disease or one or more symptoms of a disease described herein. In some embodiments, treatment may be administered after one or more signs or symptoms of the disease have developed or have been observed (i.e., therapeutic treatment). In other embodiments, treatment may be administered in the absence of signs or symptoms of the disease. For example, treatment may be administered to a susceptible subject prior to the onset of symptoms (i.e., prophylactic treatment) (e.g., in light of a history of symptoms and/or in light of exposure to a pathogen). Treatment may also be continued after symptoms have resolved, for example, to delay or prevent recurrence. In certain embodiments, treatment includes delaying the onset of at least one symptom of the disorder for a period of time.

As used herein the term “myelodysplastic syndromes” (MDS) refers to a heterogeneous group of rare hematological malignancies characterized by dysfunctional hematopoiesis, progressive cytopenia, and an increased risk of progression to acute myeloid leukemia (AML). MDS occurs when the blood-forming cells in the bone marrow become abnormal (dysplastic) and have problems making new blood cells. Many of the blood cells formed by the bone marrow cells often die or are destroyed by the body, thus leaving the individual without enough normal blood cells. Although different cell types are affected by this phenomenon, the most common finding in MDS is a shortage of red blood cells (anemia). However, hemolytic anemia has also been found to occur, albeit rarely, in patients suffering from MDS. See, for example, Leukemia Research Reports, Vol. 5, 2016, pp 23-26.

With an average age at diagnosis of 71 years, MDS patients can be severely impacted by chronically low levels of hemoglobin. This can lead to fatigue, worsening cardiopulmonary function, increased falls, and significant cognitive decline. As such, treatment of anemia is essential for overall health and quality of life. Currently, transfusions of packed red blood cells (PRBC) is the standard of care for MDS-associated anemia. The problem with transfusion dependent MDS-associated anemia patients, however, is that these patients are at a higher risk of iron overload and transfusion reactions and report a decreased quality of life.

As used herein, the terms “sickle cell disease” (SCD), “Hemoglobin SS disease”, and “sickle cell anemia” are used interchangeably. Sickle cell disease (SCD) is an inherited blood disorder that is characterized by a single-nucleotide mutation in the β-globin chain, encoding the production of an abnormal type of hemoglobin (Hb): hemoglobin S (HbS). HbS polymerizes upon deoxygenation, causing red blood cells (RBC) to sickle. Sickled RBCs are poorly deformable, which leads to vasoocclusion and hemolytic anemia. In addition, increased red cell adhesion, endothelial dysfunction, inflammation, oxidative stress, hemostatic activation, and rheological abnormalities all contribute to the complex pathophysiology of SCD. Among the factors that influence sickling are RBC metabolic intermediates, in particular, levels of 2,3-diphosphyglycerate (2,3-DPG) and adenosine triphosphate (ATP). 2,3-DPG is produced in the Rapoport-Luebering shunt, a unique RBC-specific glycolytic bypass, and serves as an important regulator of oxygen affinity of Hb. The increased intracellular 2,3-DPG levels lower oxygen affinity, thereby promoting polymerization of HbS upon deoxygenation and, hence, sickling. ATP is critical for maintaining RBC membrane integrity and deformability, and ~50% of the cell's ATP is generated in the last step of glycolysis catalyzed by pyruvate kinase (PK). Decreased levels of ATP have been reported in SCD mice, and ATP depletion has been associated with an increased number of irreversibly sickled cells.

As used herein the term “activating” means an agent, such as the crystalline and amorphous forms of Compound 1 described herein, that (measurably) increases the activity of wild type pyruvate kinase R (wt PKR) or causes wild type pyruvate kinase R (wt PKR) activity to increase to a level that is greater than wt PKR's basal levels of activity and/or an agent, such as the crystalline and amorphous forms of Compound 1 described herein, that (measurably) increases the activity of a mutant pyruvate kinase R (mPKR) or causes mutant pyruvate kinase R (mPKR) activity to increase to a level that is greater than that mutant PKR's basal levels of activity. In some embodiments the increase in activity of wtPKR or mPKR is, for example, about 20%, about 40%, about 50%, about 60%, about 70%, about 80%, about 90% or about 100% greater than the baseline activity of wtPKR or mPKR, respectively.

As used herein, the terms “red blood cells” or “RBCs”, “packed red blood cells” and “PRBCs” are used interchangeably and refer to red blood cells made from whole blood by removing the plasma. As used herein, the term “ex vivo” refers to a method that takes place outside an organism. For example, a cell (e.g., red blood cells), a tissue or blood (containing at least red blood cells, plasma and hemoglobin) may be extracted from the organism to be contacted with one or more crystalline or amorphous forms of Compound 1 as provided herein or a pharmaceutical composition thereof, optionally under artificially controlled conditions (e.g., temperature).

As used herein the term “in vitro” refers to a method that takes place outside an organism and is contained within an artificial environment. For example, a cell (e.g., red blood cells), a tissue or blood (containing at least red blood cells, plasma and hemoglobin) may be extracted from the organism to be contacted with one or more crystalline or amorphous forms of Compound 1 as provided herein or a pharmaceutical composition thereof, in a contained, artificial environment (e.g., a culture system), such as in a test tube, in a culture, in flask, in a microtiter plate, on a Petri dish, and the like.

As used herein, the terms “subject” and “patient” are used interchangeably, and mean a mammal in need of treatment, e.g., humans, companion animals (e.g., dogs, cats, and the like), farm animals (e.g., cows, pigs, horses, sheep, goats and the like) and laboratory animals (e.g., rats, mice, guinea pigs and the like). Unless stated to the contrary, a subject (or patient) is a human in need of treatment. In certain embodiments a subject is an adult human (e.g., ≥18 years of age). In other embodiments a subject is a human child (e.g., <18 years of age). In still other embodiments a subject is a human female (adult or child). In yet other embodiments a subject is a human male (adult or child). As used herein, the term subject may refer to a single subject or may refer to a plurality of subjects (i.e., two or more subjects).

In certain embodiments, a subject is a human in need of treatment of a disease, for example a disease associated with pyruvate kinase. In certain embodiments, a subject is a human in need of treatment of anemia due to MDS (e.g., very low risk MDS, low risk MDS, lower risk MDS and/or intermediate risk MDS). In certain embodiments, a subject is a human in need of treatment of sickle cell disease. In certain embodiments, a subject is a human in need of treatment of hemolytic anemia. In certain embodiments, a subject is a human in need of treatment of acquired PKD. In certain embodiments, a subject is a human in need of treatment of anemia associated with acquired PKD. In certain embodiments, a subject is a human in need of treatment of hemolytic anemia associated with acquired PKD. In other certain embodiments, a subject is a human suffering from MDS and is in need of treatment of anemia associated with acquired PKD. In still other certain embodiments, a subject is a human suffering from MDS and is in need of treatment of hemolytic anemia associated with acquired PKD.

In certain embodiments, a subject is a patient in need of regular blood transfusions (and is referred to as being “transfusion dependent” or “TD”). As used herein, the term “regular blood transfusion” refers to at least 4 transfusion episodes in a 52-week period prior to treatment with a crystalline or amorphous form of Compound 1 as disclosed herein or a composition comprising a crystalline or amorphous form of Compound 1 as disclosed herein. In certain embodiments, a regular blood transfusion refers to at least 5 transfusion episodes in a 52-week period prior to treatment with a crystalline or amorphous form of Compound 1 as disclosed herein or a composition comprising a crystalline or amorphous form of Compound 1 as disclosed herein. In certain embodiments, a regular blood transfusion refers to at least 6 transfusion episodes in a 52-week period prior to treatment with a crystalline or amorphous form of Compound 1 as disclosed herein or a composition comprising a crystalline or amorphous form of Compound 1 as disclosed herein. In certain embodiments, a regular blood transfusion refers to at least 7 transfusion episodes in a 52-week period prior to treatment with a crystalline or amorphous form of Compound 1 as disclosed herein or a composition comprising a crystalline or amorphous form of Compound 1 as disclosed herein. In certain embodiments, the subject has sickle cell disease, or anemia due to MDS (very low risk MDS, low risk MDS, lower risk MDS and/or intermediate risk MDS).

In other embodiments, a subject is referred to as being non-transfusion dependent (NTD) prior to treatment with a crystalline or amorphous form of Compound 1 as disclosed herein or a composition comprising a crystalline or amorphous form of Compound 1 as disclosed herein. As used herein, the terms “non-transfusion dependent” or “NTD” and “nontransfused” are used interchangeably and refer to subjects who do not require regular blood transfusions prior to treatment with a crystalline or amorphous form of Compound 1 as disclosed herein or a composition comprising a crystalline or amorphous form of Compound 1 as disclosed herein. In some aspects, a subject that is NTD refers to a subject that has had <3 red blood cell (RBC) units in the 16 week period before treatment with a crystalline or amorphous form of Compound 1 as disclosed herein or a composition comprising a crystalline or amorphous form of Compound 1 as disclosed herein and no transfusions in the 8 week period prior to treatment with a crystalline or amorphous form of Compound 1 as disclosed herein or a composition comprising a crystalline or amorphous form of Compound 1 as disclosed herein. In some embodiments, the subject is classified as being NTD prior to the administration of a first dose of a crystalline or amorphous form of Compound 1 as disclosed herein or a composition comprising a crystalline or amorphous form of Compound 1 as disclosed herein.

As used herein, the terms “transfusion independent” or “transfusion independence” or “transfusion free” are used interchangeably and refer to a subject that has not had an RBC transfusion for a certain period of time. In some embodiments, a subject who is transfusion independent has not had an RBC transfusion for a period of 16 consecutive weeks. In other embodiments, a subject who is transfusion independent has not had an RBC transfusion for a period of ≥8 consecutive weeks. In some embodiments, a subject becomes transfusion independent during treatment with a crystalline or amorphous form of Compound 1 as disclosed herein or a composition comprising a crystalline or amorphous form of Compound 1 as disclosed herein. In other embodiments, a subject is transfusion independent prior to treatment with a crystalline or amorphous form of Compound 1 as disclosed herein or a composition comprising a crystalline or amorphous form of Compound 1 as disclosed herein.

As used herein, the terms “high transfusion burden” or “HTB” are used interchangeably and refer to a subject that has had at least 8 RBC units over a 16 week period and has had greater than or equal to (≥) 4 transfusion episodes. In some embodiments, HTB refers to a subject that has had at least 8 RBC units over a 16 week period and has had greater than or equal to (≥) 4 transfusion episodes over the course of 8 weeks prior to treatment with a crystalline or amorphous form of Compound 1 as disclosed herein or a composition comprising a crystalline or amorphous form of Compound 1 as disclosed herein. In some embodiments, HTB refers to a subject that has had at least 8 RBC units over a 16 week period and has had greater than or equal to (≥) 4 transfusion episodes over the course of 8 weeks in the 16 weeks prior to treatment with a crystalline or amorphous form of Compound 1 as disclosed herein or a composition comprising a crystalline or amorphous form of Compound 1 as disclosed herein.

As used herein, the terms “hemoglobin response” and “Hb response” are used interchangeably and refer to an increase from a baseline Hb level (i.e., Hb concentration) of a subject, where the subject's hemoglobin response is measured over a period of time during treatment with a crystalline or amorphous form of Compound 1 as disclosed herein or a composition comprising a crystalline or amorphous form of Compound 1 as disclosed herein or following the administration of a crystalline or amorphous form of Compound 1 as disclosed herein or a composition comprising a crystalline or amorphous form of Compound 1 as disclosed herein. Unless stated to the contrary, the terms “during treatment” or “following administration” when used in connection with a crystalline or amorphous form of Compound 1 as disclosed herein or a composition comprising a crystalline or amorphous form of Compound 1 as disclosed herein, refer to ongoing treatment or administration (i.e., the subject will continue to be treated with or administered the disclosed crystalline or amorphous form of Compound 1 or composition comprising a crystalline or amorphous form of Compound 1). The terms “hemoglobin (Hb) level” and “hemoglobin concentration” are used interchangeably herein.

As used herein, the term “baseline” refers to a level or concentration that is measured or established prior to treatment or during treatment with a crystalline or amorphous form of Compound 1 as disclosed herein or a composition comprising a crystalline or amorphous form of Compound 1 as disclosed herein. For example, as used herein, the term “baseline hemoglobin level” refers to a subject's hemoglobin (Hb) level that is measured or established prior to treatment with a crystalline or amorphous form of Compound 1 as disclosed herein or a composition comprising a crystalline or amorphous form of Compound 1 as disclosed herein. In some aspects, a subject's baseline hemoglobin level (or hemoglobin concentration) may be measured or established during treatment with a crystalline or amorphous form of Compound 1 as disclosed herein or a composition comprising a crystalline or amorphous form of Compound 1 as disclosed herein. In one aspect, “hemoglobin response” refers to an increase from a baseline Hb level of the subject, where the subject's hemoglobin response is measured over a period of time during treatment. In another aspect, “hemoglobin response” refers to an increase from a baseline Hb level of the subject, where the subject's hemoglobin response is measured over a period of time following administration, e.g., for about 1 week of treatment, about 2 weeks of treatment, about 3 weeks of treatment, about 4 weeks of treatment, about 3 months of treatment, about 6 months of treatment or about 1 year of treatment or longer.

In one aspect, the hemoglobin level of the subject being treated increases from baseline over a period of at least 1 week, at least 2 weeks, at least 3 weeks, at least 4 weeks, at least 6 weeks, at least 8 weeks, at least 10 weeks, at least 12 weeks, at least 14 weeks, at least 16 weeks, at least 18 weeks, at least 20 weeks, at least 30 weeks, at least 40 weeks, or at least 50 weeks during treatment with a crystalline or amorphous form of Compound 1 as disclosed herein or a composition comprising a crystalline or amorphous form of Compound 1 as disclosed herein. In other aspects, the hemoglobin level of the subject being treated increases from baseline over a period of at least 4 weeks, at least 6 weeks, at least 8 weeks, at least 10 weeks, at least 12 weeks, at least 14 weeks, at least 16 weeks, at least 18 weeks, or at least 20 weeks during treatment with a crystalline or amorphous form of Compound 1 as disclosed herein or a composition comprising a crystalline or amorphous form of Compound 1 as disclosed herein.

The term “pharmaceutically acceptable” refers to a material that is, within the scope of sound medical judgment, suitable for use in humans without undue toxicity, irritation, allergic response, and the like, and such use is commensurate with a reasonable benefit/risk ratio.

As used herein the term “pharmaceutically acceptable excipient” or “excipient” refers to an inert substance, such as a carrier, adjuvant, additive, diluent or vehicle that does not adversely affect the pharmacological activity of the compound (including the crystalline or amorphous forms of Compound 1 as disclosed herein) with which it is formulated.

As used herein, the terms “about” and “approximately” when used in combination with a numeric value or range of values used to characterize, for example, a particular crystal form, amorphous form, or mixture thereof, or weight or a particular analytical measurement such as an endothermic event of a compound, should be understood to mean that the numeric value or range of values may deviate to an extent deemed reasonable to one of ordinary skill in the art while describing a particular crystal form, amorphous form, or mixture thereof or weight or endothermic event of a compound.

### Crystalline Forms

In a first embodiment, provided is a crystalline form of a hemi-sulfate salt of Compound 1. Alternatively, as part of a first embodiment, provided is a crystalline hemi-sulfate salt of Compound 1 having the structural formula:

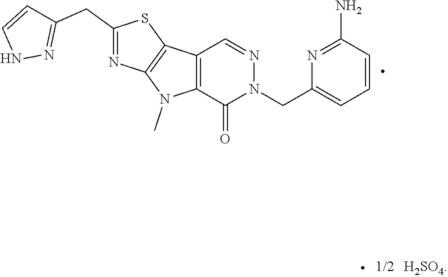 [SMILES: Cn1c2nc(Cc3cc[nH]n3)sc2c2cnn(Cc3cccc(N)n3)c(=O)c21 ; "H2SO4." ; "1/2"]

Alternatively, as part of a first embodiment, the hemi-sulfate salt described above is a solvate. In another alternative, as part of a first embodiment, the hemi-sulfate salt described above is a hydrate.

In a second embodiment, the hemi-sulfate salt described in the first embodiment is a hemi-hydrate. Alternatively, as part of a second embodiment, the hemi-sulfate salt described in the first embodiment has the structural formula:

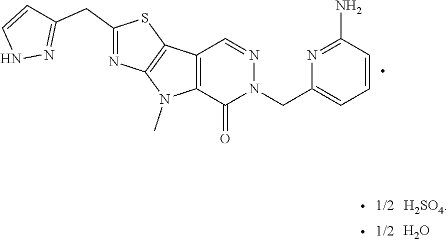

In a third embodiment, the crystalline hemi-hydrate hemi-sulfate salt of Compound 1 described herein is crystalline Form A. Alternatively, as part of a third embodiment, crystalline Form A is characterized by at least two x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°). In another alternative, as part of a third embodiment, crystalline Form A is characterized by at least three x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°). In yet another alternative, as part of a third embodiment, crystalline Form A is characterized by at least four x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°). In another alternative, as part of a third embodiment, crystalline Form A is characterized by at least five x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°). In another alternative, as part of a third embodiment, crystalline Form A is characterized by x-ray powder diffraction peaks at 20 angles selected from 9.8°(±0.2°), 11.3°(±0.2°), 13.6°(±0.2°), 18.4°(±0.2°), 22.8°(±0.2°), 23.3°(±0.2°), and 28.6°(±0.2°). In yet another alternative, as part of a third embodiment, crystalline Form A is characterized by an x-ray powder diffraction pattern comprising a peak at 2Θ angle 9.8°(±0.2°) and one or more peaks selected from 11.3°(±0.2°), 13.6°(±0.2°), 18.4°(±0.2°), 22.8°(+0.2°), 23.3°(±0.2°), and 28.6°(±0.2°). In another alternative, as part of a third embodiment, the crystalline Form A is characterized by an x-ray powder diffraction pattern comprising a peak at 2Θ angles selected from 9.8°(±0.2°) and 13.6°(±0.2°) and one or more peaks selected from 11.3°(+0.2°), 18.4°(±0.2°), 22.8°(+0.2°), 23.3°(±0.2°), and 28.6°(±0.2°). In another alternative, as part of a third embodiment, crystalline Form A is characterized by an x-ray powder diffraction pattern comprising a peak at 2Θ angles selected from 9.8°(±0.2°), 13.6°(±0.2°), and 11.3°(±0.2°), and one or more peaks selected from 18.4°(±0.2°), 22.8°(+0.2°), 23.3°(±0.2°), and 28.6°(±0.2°). In yet another alternative, as part of a third embodiment, crystalline Form A is characterized by an x-ray powder diffraction pattern comprising a peak at 2Θ angles selected from 9.8°(±0.2°), 13.6°(±0.2°), 11.3°(±0.2°), and 22.8°(±0.2°), and one or more peaks selected from 18.4°(±0.2°), 23.3°(±0.2°), and 28.6°(±0.2°). In yet another alternative, as part of a third embodiment, crystalline Form A is characterized by at least three, at least four, at least five, at least six, at least seven, at least eight or at least nine x-ray powder diffraction peaks at 2Θ angles selected from those in Table 10 (±0.2°). In yet another alternative, as part of a third embodiment, crystalline Form A is characterized by x-ray powder diffraction peaks at 2Θ angles selected from those in Table 10 (±0.2°). In a further alternative, as part of a third embodiment, the crystalline Form A is characterized by an x-ray powder diffraction pattern that is substantially the same as depicted in FIG. 1.

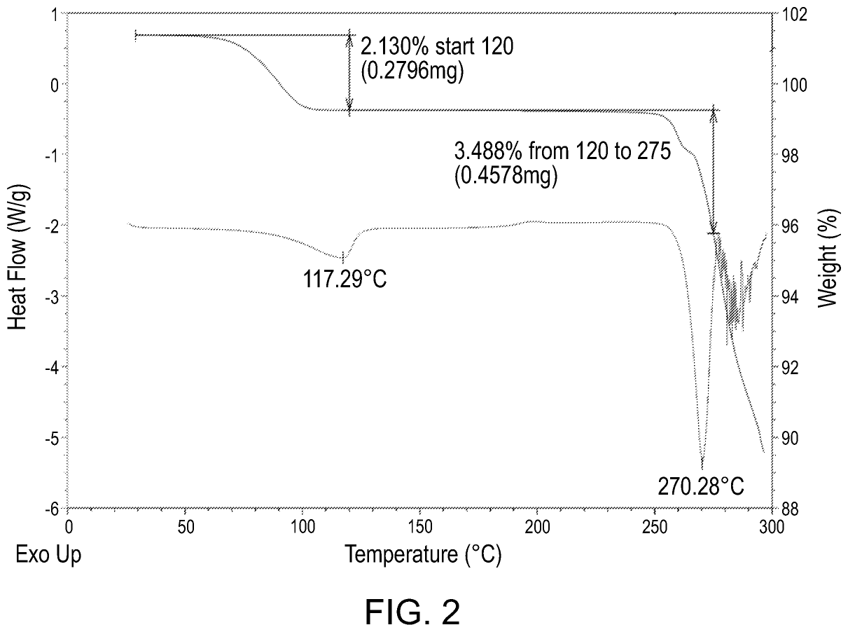

In a fourth embodiment, crystalline Form A as described in the first, second and/or third embodiments, is characterized by a differential scanning calorimetry (DSC) thermograph comprising endotherm peaks at 117° C.±5° C. and 270° C.±5° C. Alternatively, as part of a fourth embodiment, crystalline Form A as described in the first, second or third embodiments, is characterized by a differential scanning calorimetry (DSC) thermogram that is substantially similar to the one depicted in FIG. 2. In another alternative of a fourth embodiment, crystalline Form A as described in the first, second, or third embodiments, is characterized by having a moisture content of about 1% to about 4% as measured by Karl-Fischer titration.

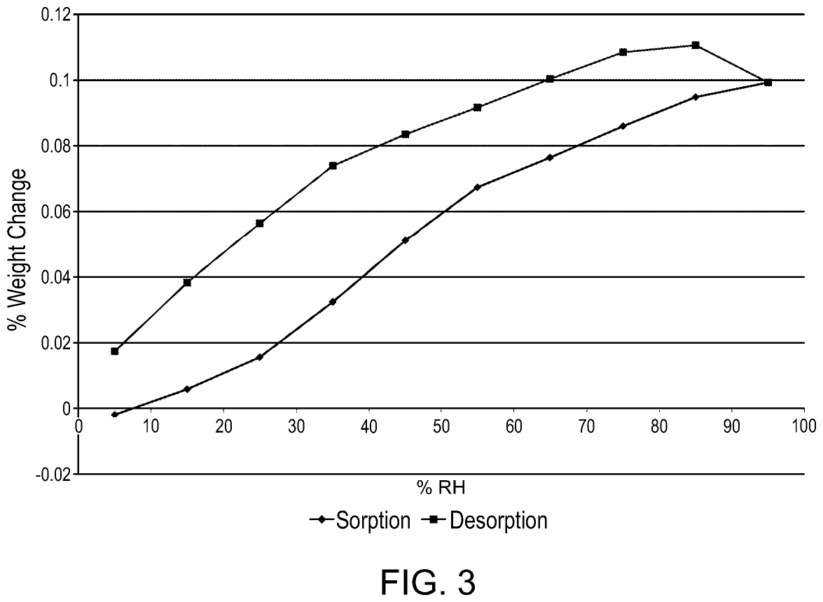

In a fifth embodiment, crystalline Form A as described in any one of the first through fourth embodiments, is characterized by a thermogravimetric analysis (TGA) thermogram comprising a weight loss of 2.1±0.5% up to 120° C.±2° C. and 3.5±0.5% from 120° C.±2° C. to 275° C.±2° C. Alternatively, as part of a fifth embodiment, crystalline Form A as described in any one of the first through fourth embodiments, is characterized by a thermogravimetric analysis/differential scanning calorimetry (TGA/DSC) thermogram that is substantially similar to the one depicted in FIG. 2. Alternatively, as part of a fifth embodiment, crystalline Form A as described in any one of the first through fourth embodiments is characterized by a DVS that is substantially similar to the one depicted in FIG. 3.

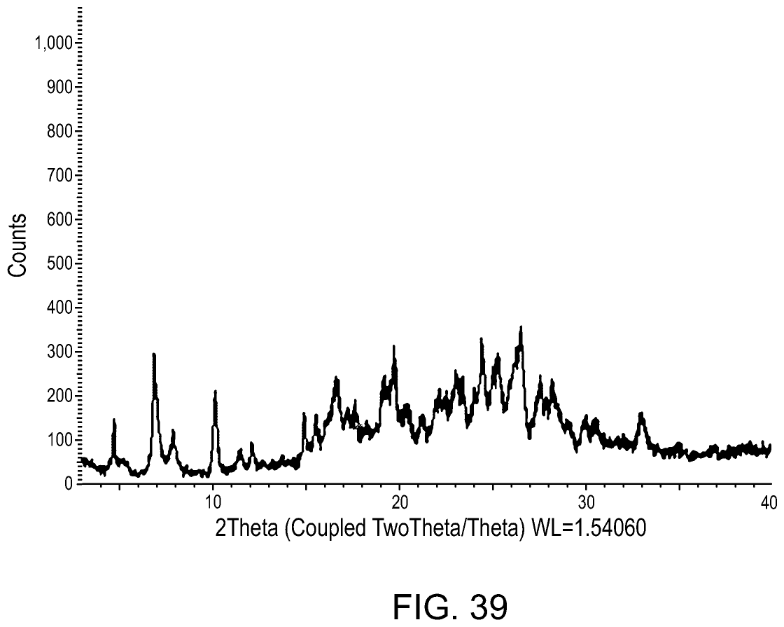

In a sixth embodiment, the crystalline hemi-hydrate hemi-sulfate salt of Compound 1 described herein is crystalline Form H. Alternatively, as part of a sixth embodiment, crystalline Form H is characterized by at least two x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°). In another alternative, as part of a sixth embodiment, crystalline Form H is characterized by at least three x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°). In yet another alternative, as part of a sixth embodiment, crystalline Form H is characterized by at least four x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°). In another alternative, as part of a sixth embodiment, crystalline Form H is characterized by at least five x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°). In another alternative, as part of a sixth embodiment, crystalline Form H is characterized by x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°). In yet another alternative, as part of a sixth embodiment, crystalline Form H is characterized by an x-ray powder diffraction pattern comprising a peak at 2Θ angle 6.9°(±0.2°) and one or more peaks selected from 10.1°(±0.2°), 16.6°(±0.2°), 19.2°(±0.2°), 19.7°(±0.2°), 24.5°(±0.2°), and 26.5°(±0.2°). In another alternative, as part of a sixth embodiment, the crystalline Form H is characterized by an x-ray powder diffraction pattern comprising a peak at 2Θ angles selected from 6.9°(±0.2°) and 26.5°(±0.2°) and one or more peaks selected from 10.1°(±0.2°), 16.6°(±0.2°), 19.2°(±0.2°), 19.7°(±0.2°), and 24.5°(±0.2°). In another alternative, as part of a sixth embodiment, crystalline Form H is characterized by an x-ray powder diffraction pattern comprising a peak at 2Θ angles selected from 6.9°(±0.2°), 24.5°(±0.2°), and 26.5°(±0.2°), and one or more peaks selected from 10.1°(±0.2°), 16.6°(±0.2°), 19.2°(±0.2°), and 19.7°(±0.2°). In yet another alternative, as part of a sixth embodiment, crystalline Form H is characterized by an x-ray powder diffraction pattern comprising a peak at 2Θ angles selected from 6.9°(±0.2°), 19.7°(±0.2°), 24.5°(±0.2°), and 26.5°(±0.2°), and one or more peaks selected from 10.1°(±0.2°), 16.6°(±0.2°), and 19.2°(±0.2°). In yet another alternative, as part of a sixth embodiment, crystalline Form H is characterized by at least three, at least four, at least five, at least six, at least seven, at least eight or at least nine x-ray powder diffraction peaks at 2Θ angles selected from those in Table 18 (±0.2°). In yet another alternative, as part of a sixth embodiment, crystalline Form H is characterized by x-ray powder diffraction peaks at 2Θ angles selected from those in Table 18 (±0.2°). Alternatively, as part of a sixth embodiment, the crystalline Form H is characterized by an x-ray powder diffraction pattern that is substantially the same as depicted in FIG. 39.

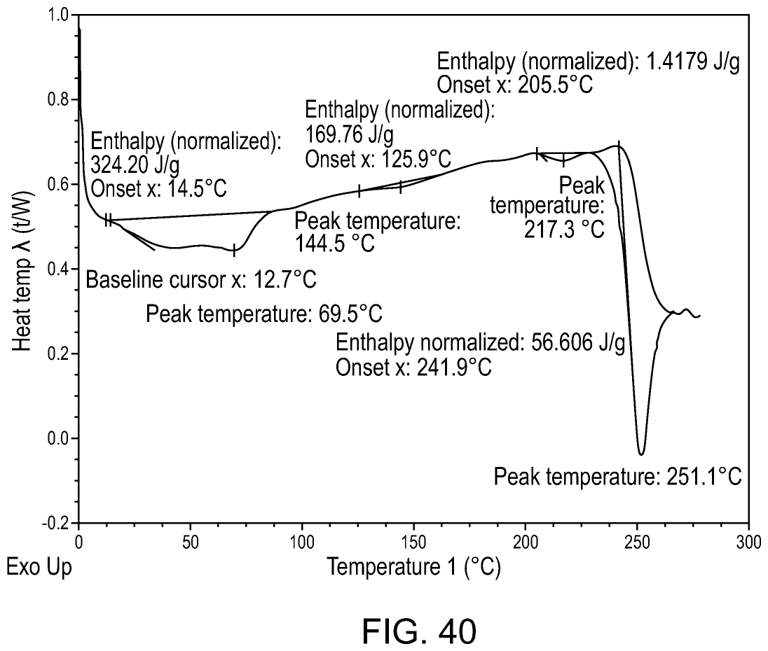

In a seventh embodiment, crystalline Form H as described in the first and sixth embodiments, is characterized by a differential scanning calorimetry (DSC) thermograph comprising endotherm peaks at 126° C.±5° C. and 206° C.±5° C. Alternatively, as part of a fourth embodiment, crystalline Form H as described in the first or sixth embodiments, is characterized by a differential scanning calorimetry (DSC) thermogram that is substantially similar to the one depicted in FIG. 40.

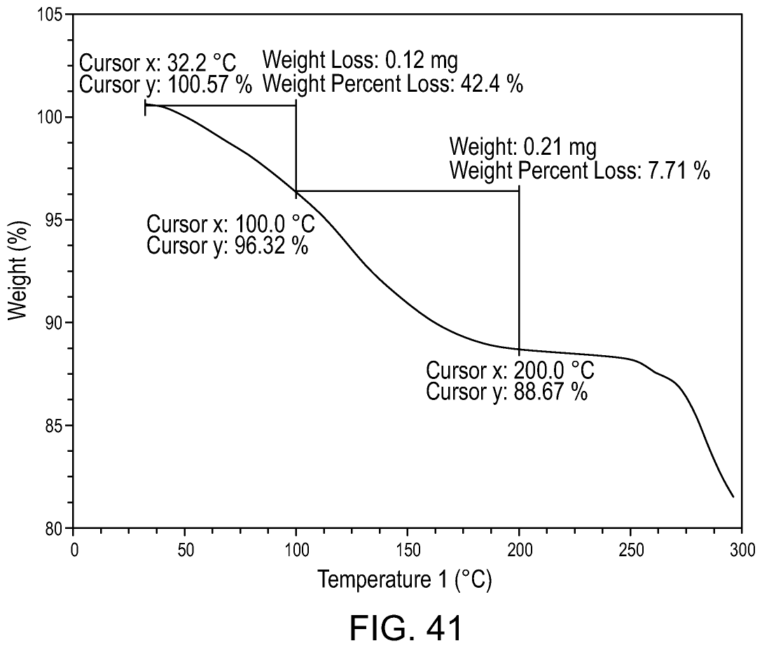

In an eighth embodiment, crystalline Form H as described in a first, sixth, or seventh embodiment, is characterized by a thermogravimetric analysis (TGA) thermogram comprising a weight loss of 4.2±0.5% up to 100° C.±2° C. and 7.7±0.5% from 200° C.±2° C. Alternatively, as part of an eighth embodiment, crystalline Form H as described in any one of the first, sixth, or seventh embodiments, is characterized by a thermogravimetric analysis (TGA) thermogram that is substantially similar to the one depicted in FIG. 41.

In a ninth embodiment, provided is a crystalline form of a phosphate salt of Compound 1. Alternatively, as part of a ninth embodiment, provided herein is a crystalline phosphate salt of Compound 1 having the structural formula:

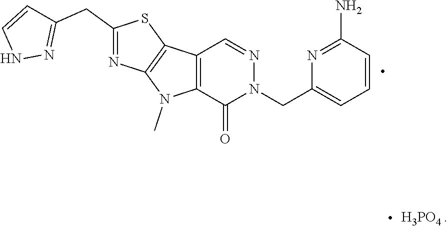 [SMILES: Cn1c2nc(Cc3cc[nH]n3)sc2c2cnn(Cc3cccc(N)n3)c(=O)c21 ; "H3PO4 ."]

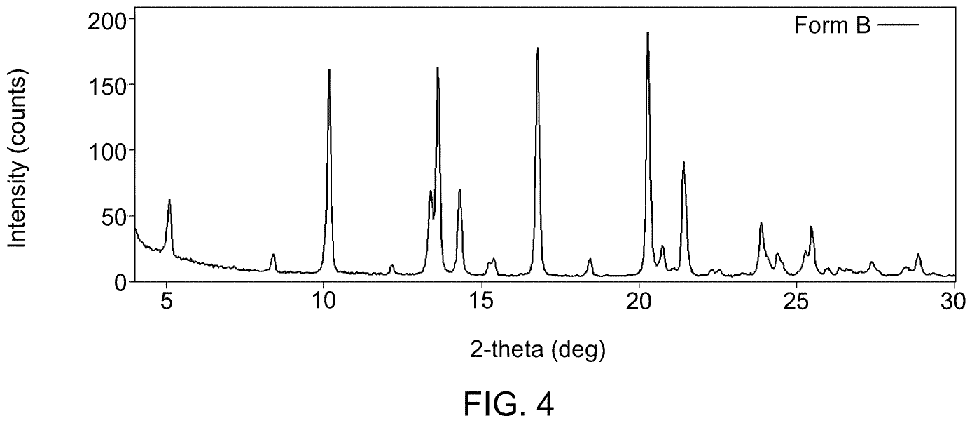

In a tenth embodiment, the crystalline phosphate salt described herein is crystalline Form B. Alternatively, as part of a tenth embodiment, crystalline Form B is characterized by at least two x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°). In another alternative, as part of a tenth embodiment, crystalline Form B is characterized by at least three x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°). In another alternative, as part of a tenth embodiment, crystalline Form B is characterized by at least four x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°). In another alternative, as part of a tenth embodiment, crystalline Form B is characterized by at least five x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°). In another alternative, as part of a tenth embodiment, crystalline Form B is characterized by at least six x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°). In another alternative, as part of a tenth embodiment, crystalline Form B is characterized by x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°). In another alternative, as part of a tenth embodiment, crystalline Form B is characterized by an x-ray powder diffraction pattern comprising a peak at 2Θ angle 20.3°(±0.2°) and one or more peaks selected from 10.2°(±0.2°), 13.4°(±0.2°), 13.6°(±0.2°), 14.3°(±0.2°), 16.8°(±0.2°), and 21.4°(±0.2°). In another alternative, as part of a tenth embodiment, crystalline Form B is characterized by an x-ray powder diffraction pattern comprising a peak at a 2Θ angle selected from 20.3°(±0.2°) and 16.8°(±0.2°), and one or more peaks selected from 10.2°(±0.2°), 13.4°(±0.2°), 13.6°(±0.2°), 14.3°(±0.2°), and 21.4°(±0.2°). In another alternative, as part of a tenth embodiment, crystalline Form B is characterized by an x-ray powder diffraction pattern comprising a peak at a 2Θ angle selected from 20.3°(±0.2°), 16.8° (±0.2°), and 13.6°(±0.2°), and one or more peaks selected from 10.2°(±0.2°), 13.4°(±0.2°), 14.3°(±0.2°), and 21.4°(±0.2°). In another alternative, as part of a tenth embodiment, crystalline Form B is characterized by an x-ray powder diffraction pattern comprising a peak at a 2Θ angle selected from 20.3°(±0.2°), 16.8°(±0.2°), 13.6°(±0.2°), and 10.2°(±0.2°), and one or more peaks selected from 13.4°(±0.2°), 14.3°(±0.2°), and 21.4°(±0.2°). In yet another alternative, as part of a tenth embodiment, crystalline Form B is characterized by at least three, at least four, at least five, at least six, at least seven, at least eight or at least nine x-ray powder diffraction peaks at 2Θ angles selected from those in Table 11 (±0.2°). In yet another alternative, as part of a tenth embodiment, crystalline Form B is characterized by x-ray powder diffraction peaks at 2Θ angles selected from those in Table 11 (±0.2°). In a further alternative, as part of a tenth embodiment, crystalline Form B is characterized by an x-ray powder diffraction pattern that is substantially the same as depicted in FIG. 4.

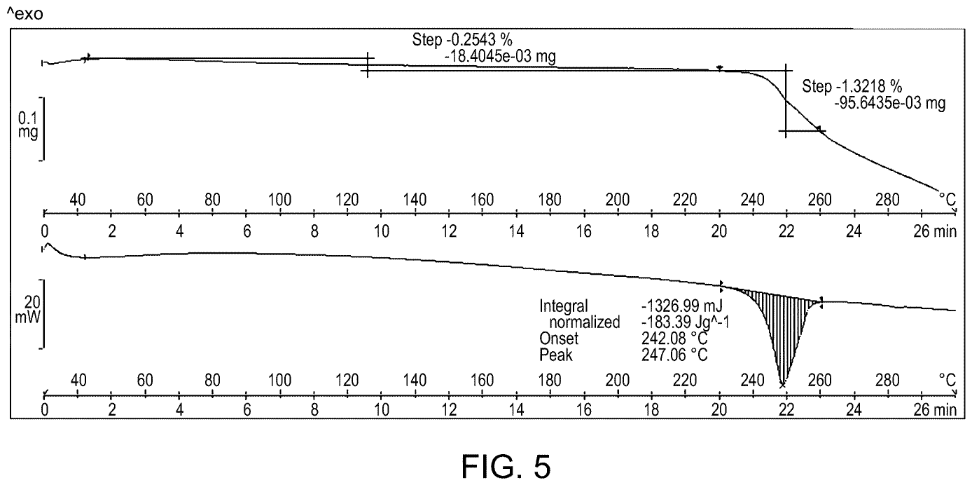

In an eleventh embodiment, crystalline Form B, as described in the ninth or tenth embodiment, is characterized by a differential scanning calorimetry (DSC) thermograph comprising an endotherm peak at 247° C.±5° C. Alternatively, as part of an eleventh embodiment, crystalline Form B, as described in the ninth or tenth embodiment, is characterized by a differential scanning calorimetry (DSC) thermogram that is substantially similar to the one depicted in FIG. 5.

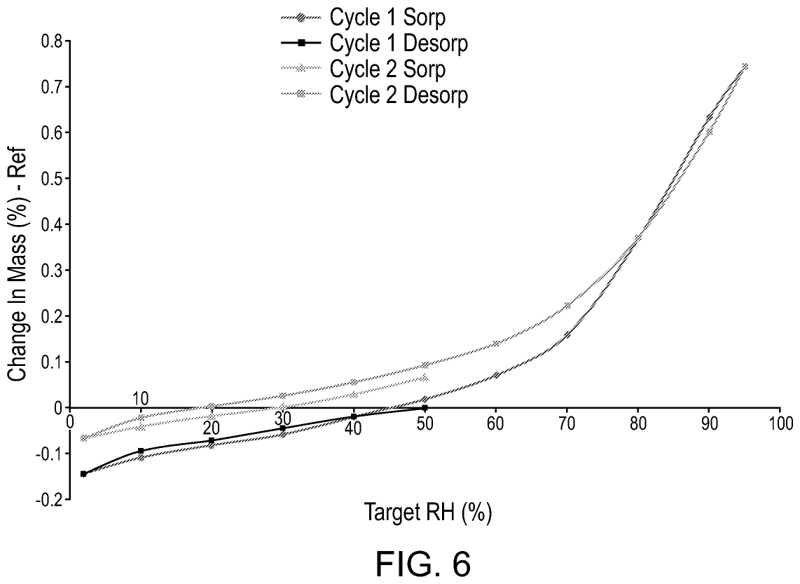

In a twelfth embodiment, crystalline Form B, as described in any one of the ninth, tenth, or eleventh embodiments, is characterized by a thermogravimetric analysis (TGA) thermogram comprising a weight loss of 1.5±0.5% up to 260° C.±2° C. Alternatively, as part of a twelfth embodiment, crystalline Form B, as described in any one of the ninth, tenth, or eleventh embodiments, is characterized by a thermogravimetric analysis (TGA) thermogram that is substantially similar to the one depicted in FIG. 5. In another alternative, as part of a twelfth embodiment, crystalline Form B as described in the ninth or tenth embodiments, is characterized by having a moisture content of about 3% to about 6% as measured by Karl-Fischer titration. In another alternative, as part of a twelfth embodiment, crystalline Form B, as described in any one of the ninth, tenth, or eleventh embodiments, is characterized by a DVS that is substantially similar to the one depicted in FIG. 6.

In a thirteenth embodiment, is provided a crystalline form of a tartrate salt of Compound 1. Alternatively, as part of a thirteenth embodiment, is provided a crystalline form of a DL-tartrate salt of Compound 1. In another alternative, as part of a thirteenth embodiment, is provided a crystalline form of a L-tartrate salt of Compound 1. In another alternative, as part of a thirteenth embodiment, is provided a crystalline form of a DL-tartrate salt of Compound 1 having the structural formula:

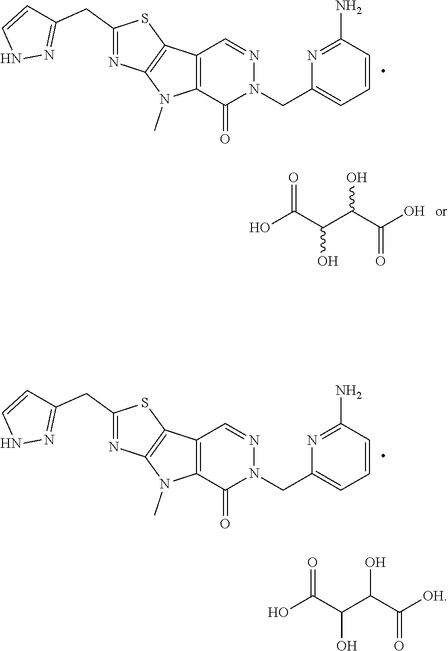 [SMILES: Cn1c2nc(Cc3cc[nH]n3)sc2c2cnn(Cc3cccc(N)n3)c(=O)c21 ; O=C(O)C(O)C(O)C(=O)O ; "or" ; Cn1c2nc(Cc3cc[nH]n3)sc2c2cnn(Cc3cccc(N)n3)c(=O)c21 ; O=C(O)C(O)C(O)C(=O)O]

In another alternative, as part of a thirteenth embodiment, is provided a crystalline form of a L-tartrate salt of Compound 1 having the structural formula:

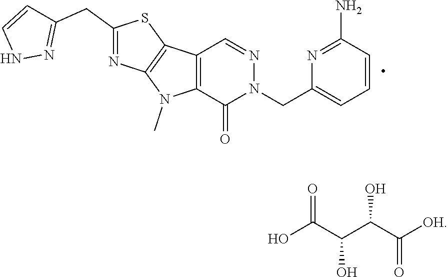 [SMILES: Cn1c2nc(Cc3cc[nH]n3)sc2c2cnn(Cc3cccc(N)n3)c(=O)c21 ; O=C(O)[C@@H](O)[C@H](O)C(=O)O]

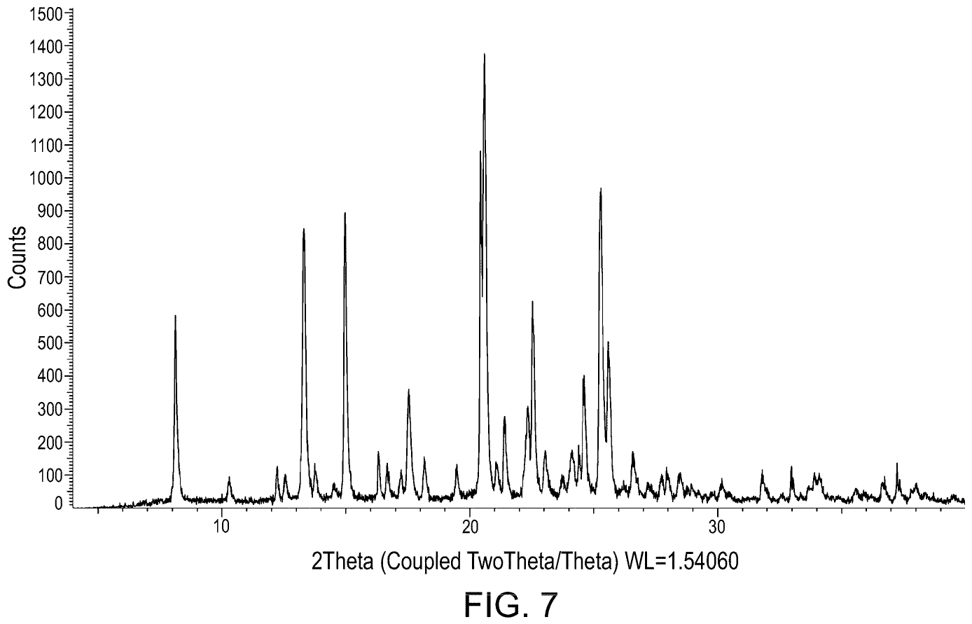

In a fourteenth embodiment, is provided a crystalline DL-tartrate salt of Compound 1 described herein as crystalline Form C. Alternatively, as part of a fourteenth embodiment, crystalline Form C is characterized by at least two x-ray powder diffraction peaks at 2Θ angles selected from 8.1°(±0.2°), 13.3°(±0.2°), 15.0°(±0.2°), 20.4°(±0.2°), 20.6° (±0.2°), 22.6°(±0.2°), and 25.3°(±0.2°). In another alternative, as part of a fourteenth embodiment, crystalline Form C is characterized by at least three x-ray powder diffraction peaks at 2Θ angles selected from 8.1°(±0.2°), 13.3°(±0.2°), 15.0°(±0.2°), 20.4°(±0.2°), 20.6° (±0.2°), 22.6°(±0.2°), and 25.3°(±0.2°). In another alternative, as part of a fourteenth embodiment, crystalline Form C is characterized by at least four x-ray powder diffraction peaks at 2Θ angles selected from 8.1°(±0.2°), 13.3°(±0.2°), 15.0°(±0.2°), 20.4°(±0.2°), 20.6° (±0.2°), 22.6°(±0.2°), and 25.3°(±0.2°). In another alternative, as part of a fourteenth embodiment, crystalline Form C is characterized by at least five x-ray powder diffraction peaks at 2Θ angles selected from 8.1°(±0.2°), 13.3°(±0.2°), 15.0°(±0.2°), 20.4°(±0.2°), 20.6° (±0.2°), 22.6°(±0.2°), and 25.3°(±0.2°). In another alternative, as part of a fourteenth embodiment, crystalline Form C is characterized by at least six x-ray powder diffraction peaks at 2Θ angles selected from 8.1°(±0.2°), 13.3°(±0.2°), 15.0°(±0.2°), 20.4°(±0.2°), 20.6° (±0.2°), 22.6°(±0.2°), and 25.3°(±0.2°). In another alternative, as part of a fourteenth embodiment, crystalline Form C is characterized by x-ray powder diffraction peaks at 2Θ angles selected from 8.1°(±0.2°), 13.3°(±0.2°), 15.0°(±0.2°), 20.4°(±0.2°), 20.6°(±0.2°), 22.6°(±0.2°), and 25.3°(±0.2°). In another alternative, as part of a fourteenth embodiment, crystalline Form C is characterized by an x-ray powder diffraction pattern comprising a peak at 2Θ angle 20.6°(±0.2°) and one or more peaks selected from 8.1°(±0.2°), 13.3°(±0.2°), 15.0°(±0.2°), 20.4°(±0.2°), 22.6°(±0.2°), and 25.3°(±0.2°). In another alternative, as part of a fourteenth embodiment, crystalline Form C is characterized by an x-ray powder diffraction pattern comprising a peak at a 2Θ angle selected from 20.6°(±0.2°) and 20.4°(±0.2°), and one or more peaks selected from 8.1°(±0.2°), 13.3°(±0.2°), 15.0°(±0.2°), 22.6°(±0.2°), and 25.3°(±0.2°). In another alternative, as part of a fourteenth embodiment, crystalline Form C is characterized by an x-ray powder diffraction pattern comprising a peak at a 2Θ angle selected from 20.6°(±0.2°), 20.4°(±0.2°), and 25.3°(±0.2°), and one or more peaks selected from 8.1°(±0.2°), 13.3°(±0.2°), 15.0°(±0.2°), and 22.6°(±0.2°). In another alternative, as part of a fourteenth embodiment, crystalline Form C is characterized by an x-ray powder diffraction pattern comprising a peak at a 2Θ angle selected from 20.6°(0.2°), 20.4°(±0.2°), 25.3°(±0.2°), and 15.0°(±0.2°), and one or more peaks selected from 8.1°(±0.2°), 13.3°(±0.2°), and 22.6°(±0.2°). In another alternative, as part of a fourteenth embodiment, crystalline Form C is characterized by at least three, at least four, at least five, at least six, at least seven, at least eight or at least nine x-ray powder diffraction peaks at 2Θ angles selected from those in Table 12 (±0.2°). In another alternative, as part of a fourteenth embodiment, crystalline Form C is characterized by x-ray powder diffraction peaks at 2Θ angles selected from those in Table 12 (±0.2°). In another alternative, as part of a fourteenth embodiment, crystalline Form C is characterized by an x-ray powder diffraction pattern that is substantially the same as depicted in FIG. 7.

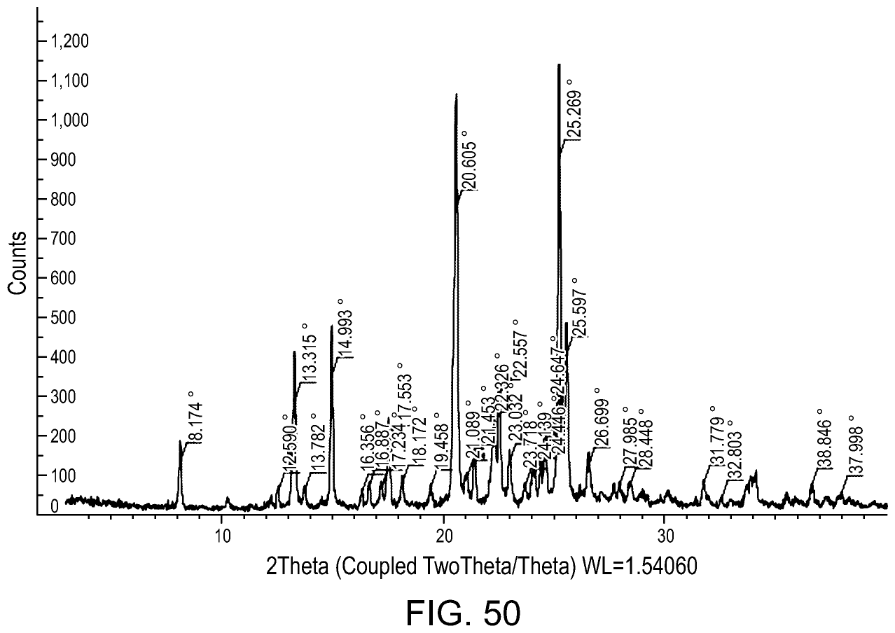

In an alternative to the fourteenth embodiment, provided is an alternate crystalline DL-tartrate salt of Compound 1 described herein as crystalline Form C-1. Alternatively, as part of a fourteenth embodiment, crystalline Form C-1 is characterized by at least two x-ray powder diffraction peaks at 2Θ angles selected from 13.3°(±0.2°),14.9°(±0.2°), 20.6°(±0.2°), 22.3°(±0.2°), 22.5°(±0.2°), 25.26°(±0.2°), and 25.6°(±0.2°). In another alternative, as part of a fourteenth embodiment, crystalline Form C-1 is characterized by at least three x-ray powder diffraction peaks at 2Θ angles selected from 13.3°(±0.2°),14.9°(±0.2°), 20.6°(±0.2°), 22.3°(±0.2°), 22.5°(±0.2°), 25.26°(±0.2°), and 25.6°(±0.2°). In another alternative, as part of a fourteenth embodiment, crystalline Form C-1 is characterized by at least four x-ray powder diffraction peaks at 2Θ angles selected from 13.3°(±0.2°),14.9°(±0.2°), 20.6°(±0.2°), 22.3°(±0.2°), 22.5°(±0.2°), 25.26°(±0.2°), and 25.6°(±0.2°). In another alternative, as part of a fourteenth embodiment, crystalline Form C-1 is characterized by at least five x-ray powder diffraction peaks at 2Θ angles selected from 13.3°(±0.2°),14.9°(±0.2°), 20.6°(±0.2°), 22.3°(±0.2°), 22.5°(±0.2°), 25.26°(±0.2°), and 25.6°(±0.2°). In another alternative, as part of a fourteenth embodiment, crystalline Form C-1 is characterized by at least six x-ray powder diffraction peaks at 2Θ angles selected from 13.3°(±0.2°),14.9°(±0.2°), 20.6°(±0.2°), 22.3°(±0.2°), 22.5°(±0.2°), 25.26°(±0.2°), and 25.6°(±0.2°). In another alternative, as part of a fourteenth embodiment, crystalline Form C-1 is characterized by x-ray powder diffraction peaks at 2Θ angles selected from 13.3°(±0.2°),14.9°(±0.2°), 20.6°(±0.2°), 22.3°(±0.2°), 22.5°(±0.2°), 25.26°(±0.2°), and 25.6°(±0.2°). In another alternative, as part of a fourteenth embodiment, crystalline Form C-1 is characterized by an x-ray powder diffraction pattern comprising a peak at 2Θ angle 25.26°(±0.2°) and one or more peaks selected from 13.3°(±0.2°),14.9°(±0.2°), 20.6°(±0.2°), 22.3°(±0.2°), 22.5°(±0.2°), and 25.6°(±0.2°). In another alternative, as part of a fourteenth embodiment, crystalline Form C-1 is characterized by an x-ray powder diffraction pattern comprising a peak at a 2Θ angle selected from 20.6°(±0.2°) and 25.26°(±0.2°), and one or more peaks selected from 13.3°(±0.2°),14.9°(±0.2°), 22.3°(±0.2°), 22.5°(±0.2°), and 25.6°(±0.2°). In another alternative, as part of a fourteenth embodiment, crystalline Form C-1 is characterized by an x-ray powder diffraction pattern comprising a peak at a 2Θ angle selected from 20.6°(±0.2°), 25.26°(±0.2°), and 25.6°(±0.2°), and one or more peaks selected from 13.3°(±0.2°),14.9°(±0.2°), 22.3°(±0.2°), and 22.5°(±0.2°). In another alternative, as part of a fourteenth embodiment, crystalline Form C-1 is characterized by an x-ray powder diffraction pattern comprising a peak at a 2Θ angle selected from 14.9°(±0.2°), 20.6°(±0.2°), 25.26°(±0.2°), and 25.6°(±0.2°), and one or more peaks selected from 13.3°(±0.2°), 22.3°(±0.2°), and 22.5°(±0.2°). In another alternative, as part of a fourteenth embodiment, crystalline Form C-1 is characterized by at least three, at least four, at least five, at least six, at least seven, at least eight or at least nine x-ray powder diffraction peaks at 2Θ angles selected from those in Table 23 (±0.2°). In another alternative, as part of a fourteenth embodiment, crystalline Form C-1 is characterized by x-ray powder diffraction peaks at 2Θ angles selected from those in Table 23 (±0.2°). In another alternative, as part of a fourteenth embodiment, crystalline Form C-1 is characterized by an x-ray powder diffraction pattern that is substantially the same as depicted in FIG. 50.

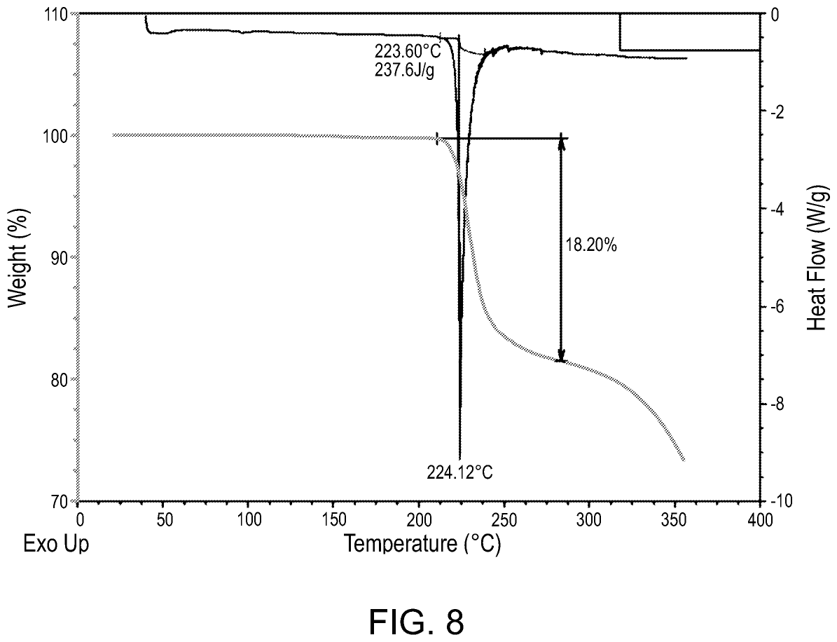

In a fifteenth embodiment, crystalline Form C, as described in the thirteenth or fourteenth embodiment, is characterized by a differential scanning calorimetry (DSC) thermograph comprising an endotherm peak at 224° C.±5° C. Alternatively, as part of the twelfth embodiment, crystalline Form C, as described in the thirteenth or fourteenth embodiment, is characterized by a differential scanning calorimetry (DSC) thermogram that is substantially similar to the one depicted in FIG. 8.

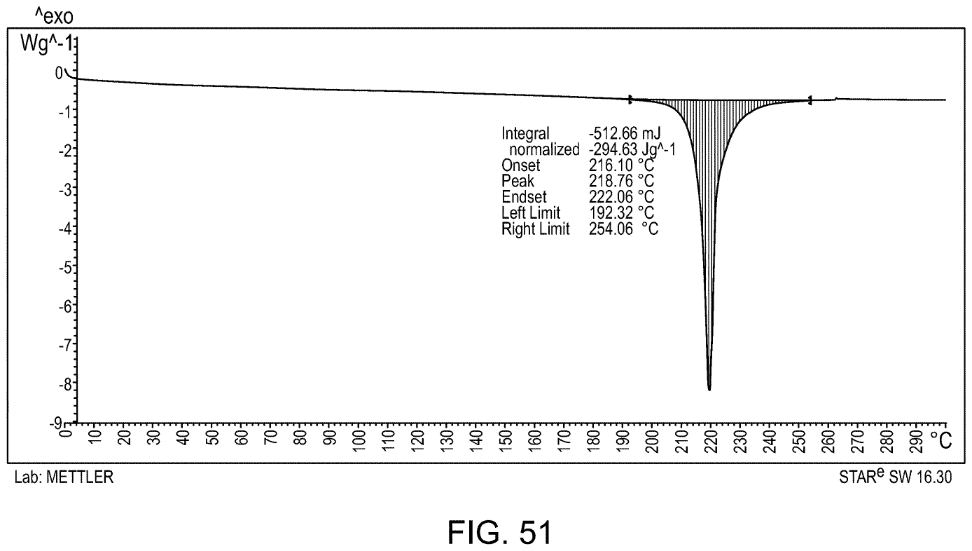

In an alternative to the fifteenth embodiment, crystalline Form C-1, as described in the thirteenth or fourteenth embodiment, is characterized by a differential scanning calorimetry (DSC) thermograph comprising an endotherm peak at 218.76° C.±5° C. Alternatively, as part of the twelfth embodiment, crystalline Form C-1, as described in the thirteenth or fourteenth embodiment, is characterized by a differential scanning calorimetry (DSC) thermogram that is substantially similar to the one depicted in FIG. 51.

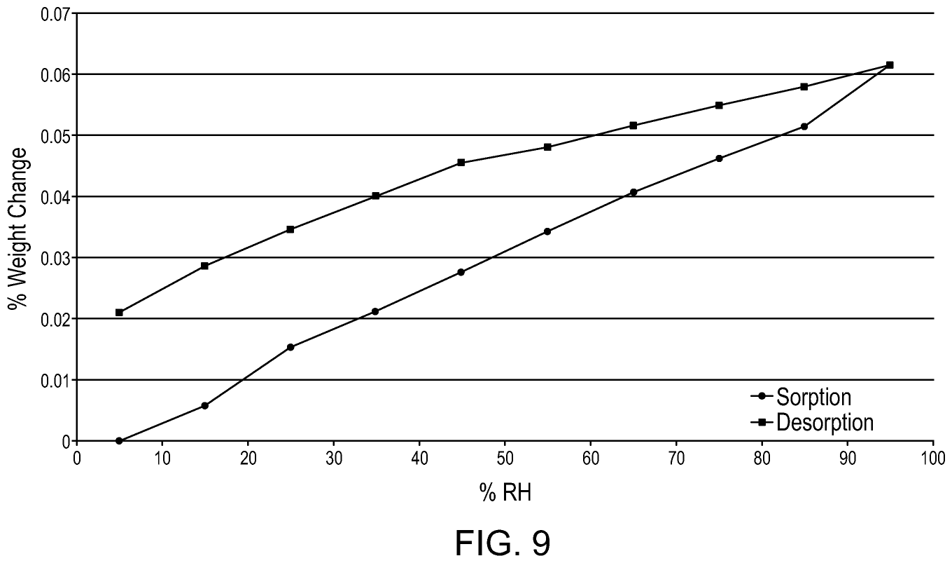

In a sixteenth embodiment, crystalline Form C, as described in any one of the thirteenth, fourteenth, or fifteenth embodiments, is characterized by a thermogravimetric analysis (TGA) thermogram comprising a weight loss of 18.2±0.5% up to 290° C.±2° C. Alternatively, as part of a sixteenth embodiment, crystalline Form C, as described in any one of the thirteenth, fourteenth, or fifteenth embodiments, is characterized by a thermogravimetric analysis (TGA) thermogram that is substantially similar to the one depicted in FIG. 8. Alternatively, as part of a sixteenth embodiment, crystalline Form C, as described in any one of the thirteenth, fourteenth, or fifteenth embodiments, is characterized by a DVS that is substantially similar to the one depicted in FIG. 9. In another alternative, as part of a sixteenth embodiment, crystalline Form C, as described in the thirteenth, fourteenth, or fifteenth embodiments, is characterized by having a moisture content of about 0.05% to about 2% as measured by Karl-Fischer titration.

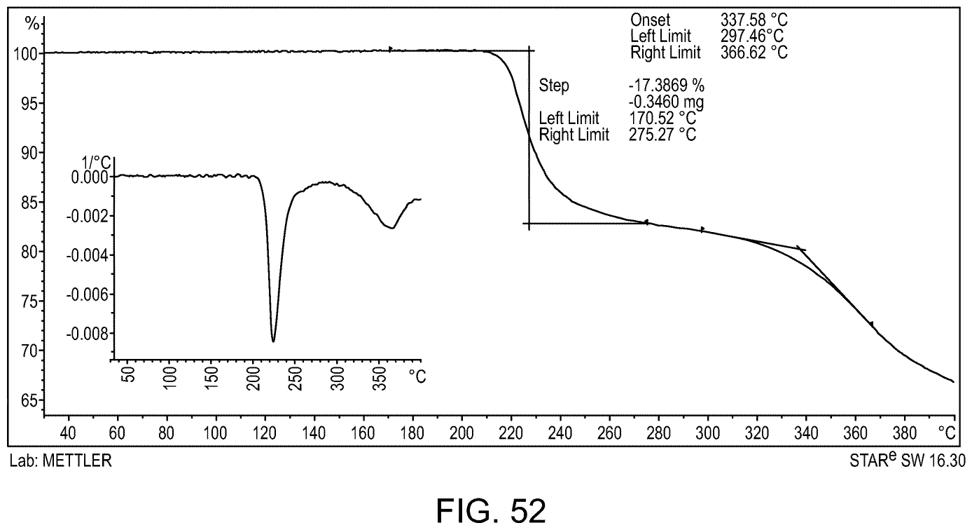

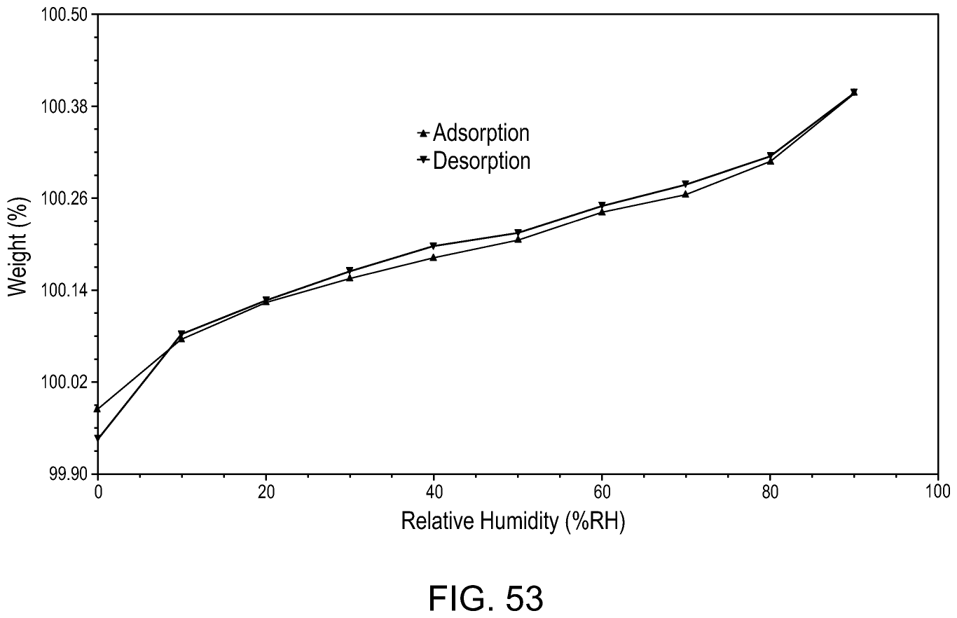

In an alternative to the sixteenth embodiment, crystalline Form C-1, as described in any one of the thirteenth, fourteenth, or fifteenth embodiments, is characterized by a thermogravimetric analysis (TGA) thermogram comprising a weight loss of 17.4±0.5% up to 275° C.±2° C. Alternatively, as part of a sixteenth embodiment, crystalline Form C-1, as described in any one of the thirteenth, fourteenth, or fifteenth embodiments, is characterized by a thermogravimetric analysis (TGA) thermogram that is substantially similar to the one depicted in FIG. 52. Alternatively, as part of a sixteenth embodiment, crystalline Form C-1, as described in any one of the thirteenth, fourteenth, or fifteenth embodiments, is characterized by a DVS that is substantially similar to the one depicted in FIG. 53.

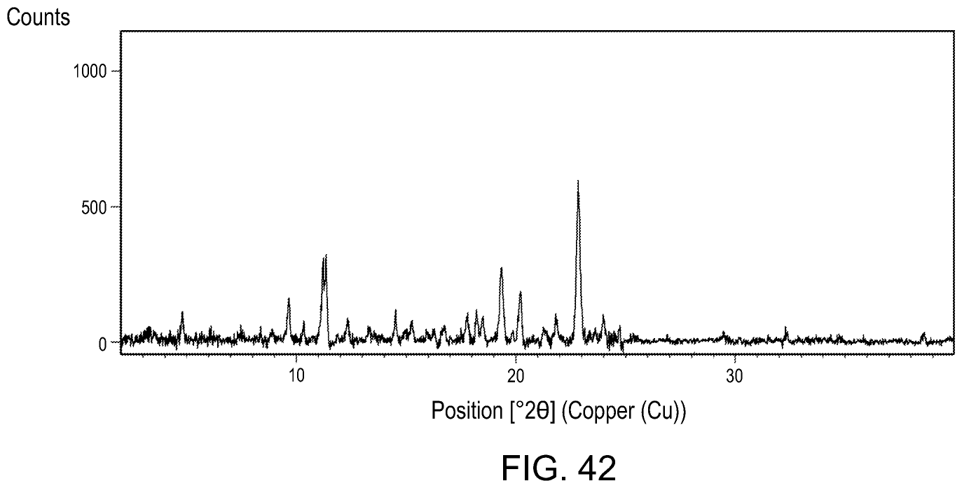

In a seventeenth embodiment, is provided a crystalline DL-tartrate salt described herein as crystalline Form F. Alternatively, as part of a seventeenth embodiment, crystalline Form F is characterized by at least two x-ray powder diffraction peaks at 2Θ angles selected from 4.8°(±0.2°), 9.60(±0.2°), 11.3°(±0.2°), 14.5°(±0.2°), 18.2°(±0.2°), 19.3°(±0.2°), 20.2°(±0.2°), and 22.8°(±0.2°). In another alternative, as part of a seventeenth embodiment, crystalline Form F is characterized by at least three x-ray powder diffraction peaks at 20 angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°). In another alternative, as part of a seventeenth embodiment, crystalline Form F is characterized by at least four x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°). In an alternative, as part of a seventeenth embodiment, crystalline Form F is characterized by at least five x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°). In another alternative, as part of a seventeenth embodiment, crystalline Form F is characterized by at least six x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°). In yet another alternative, as part of a seventeenth embodiment, crystalline Form F is characterized by x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°). In another alternative, as part of a seventeenth embodiment, crystalline Form F is characterized by an x-ray powder diffraction pattern comprising a peak at 2Θ angle 22.8°(±0.2°) and one or more peaks selected from 4.8°(±0.2°), 9.6°(±0.2°), 11.3°(±0.2°), 14.5°(±0.2°), 18.2°(±0.2°), 19.3°(±0.2°), and 20.2°(±0.2°). In yet another alternative, as part of a seventeenth embodiment, crystalline Form F is characterized by an x-ray powder diffraction pattern comprising a peak at a 2Θ angle selected from 19.3°(±0.2°) and 22.8°(±0.2°), and one or more peaks selected from 4.8°(±0.2°), 9.6°(±0.2°), 11.3°(±0.2°), 14.5°(±0.2°), 18.2°(±0.2°), and 20.2°(±0.2°). In another alternative, as part of a seventeenth embodiment, crystalline Form F is characterized by an x-ray powder diffraction pattern comprising a peak at a 2Θ angle selected from 19.3°(±0.2°) 20.2°(±0.2°), and 22.8°(±0.2°), and one or more peaks selected from 4.8°(±0.2°), 9.6°(±0.2°), 11.3°(±0.2°), 14.5°(±0.2°), and 18.2°(±0.2°). In another alternative, as part of the seventeenth embodiment, crystalline Form F is characterized by an x-ray powder diffraction pattern comprising a peak at a 2Θ angle selected from 9.6°(±0.2°), 19.3°(±0.2°) 20.2°(±0.2°), and 22.8°(±0.2°), and one or more peaks selected from 4.8°(±0.2°), 11.3°(±0.2°), 14.5°(±0.2°), and 18.2°(±0.2°). In yet another alternative, as part of a seventeenth embodiment, crystalline Form F is characterized by at least three, at least four, at least five, at least six, at least seven, at least eight or at least nine x-ray powder diffraction peaks at 2Θ angles selected from those in Table 19 (±0.2°). In yet another alternative, as part of a seventeenth embodiment, crystalline Form F is characterized by x-ray powder diffraction peaks at 2Θ angles selected from those in Table 19 (±0.2°). In yet another alternative, as part of a seventeenth embodiment, crystalline Form F is characterized by an x-ray powder diffraction pattern that is substantially the same as depicted in FIG. 42.

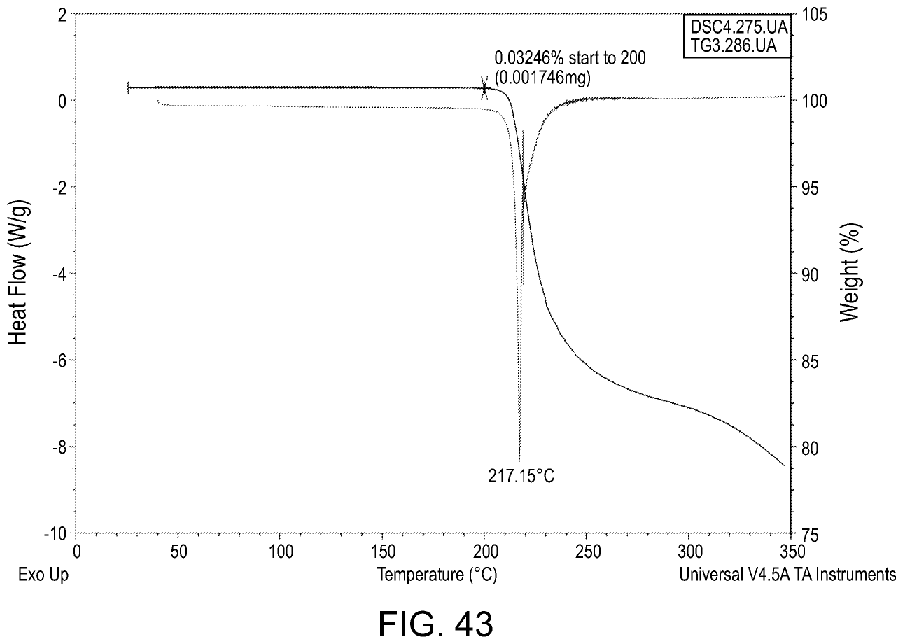

In an eighteenth embodiment, crystalline Form F, as described in the thirteenth or seventeenth embodiments, is characterized by a differential scanning calorimetry (DSC) thermograph comprising an endotherm peak at 217° C.±5° C. Alternatively, as part of an eighteenth embodiment, crystalline Form F, as described in the thirteenth or seventeenth embodiment, is characterized by a differential scanning calorimetry (DSC) thermogram that is substantially similar to the one depicted in FIG. 43.

In a nineteenth embodiment, crystalline Form F, as described in any one of the thirteenth, seventeenth, or eighteenth embodiments, is characterized by a thermogravimetric analysis (TGA) thermogram comprising a weight loss of 0.03±0.01% up to 200° C.±2° C. Alternatively, as part of a nineteenth embodiment, crystalline Form F, as described in the thirteenth, seventeenth, or eighteenth embodiments, is characterized by a thermogravimetric analysis (TGA) thermogram that is substantially similar to the one depicted in FIG. 43.

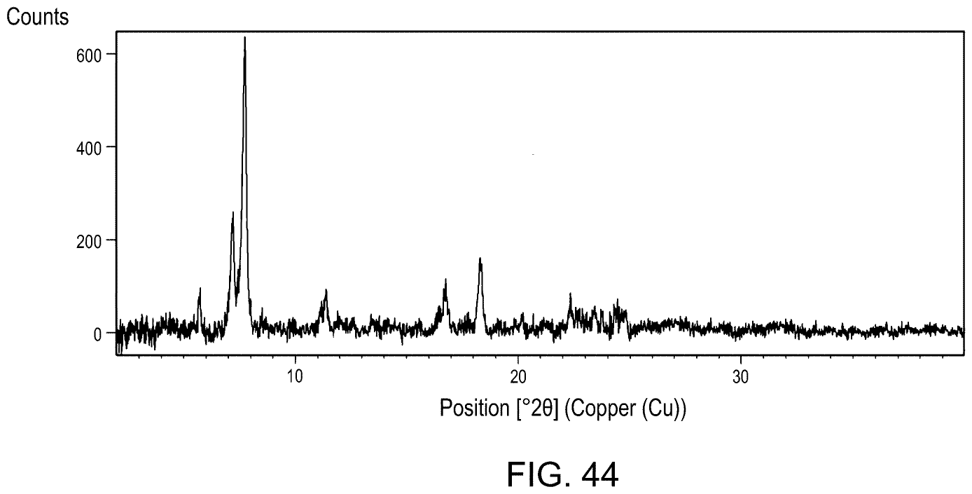

In a twentieth embodiment, is provided a crystalline DL-tartrate salt described herein as crystalline Form G. Alternatively, as part of a twentieth embodiment, crystalline Form G is characterized by at least two x-ray powder diffraction peaks at 2Θ angles selected from 5.7°(±0.2°), 7.20(±0.2°), 7.7°(±0.2°), 11.3°(±0.2°), 16.8°(±0.2°), 18.3°(±0.2°), and 20.1°(±0.2°). In another alternative, as part of a twentieth embodiment, crystalline Form G is characterized by at least three x-ray powder diffraction peaks at 2Θ angles selected from 5.7°(±0.2°), 7.2(±0.2°), 7.7°(±0.2°), 11.3°(±0.2°), 16.8°(±0.2°), 18.3°(±0.2°), and 20.1°(±0.2°). In another alternative, as part of a twentieth embodiment, crystalline Form G is characterized by at least four x-ray powder diffraction peaks at 2Θ angles selected from 5.7°(±0.2°), 7.2(±0.2°), 7.7°(±0.2°), 11.3°(±0.2°), 16.8°(±0.2°), 18.3°(±0.2°), and 20.1°(±0.2°). In an alternative, as part of a twentieth embodiment, crystalline Form G is characterized by at least five x-ray powder diffraction peaks at 2Θ angles selected from 5.7°(±0.2°), 7.2(±0.2°), 7.7°(±0.2°), 11.3°(±0.2°), 16.8°(±0.2°), 18.3°(±0.2°), and 20.1°(±0.2°). In another alternative, as part of a twentieth embodiment, crystalline Form G is characterized by at least six x-ray powder diffraction peaks at 2Θ angles selected from 5.7(±0.2°), 7.2°(±0.2°), 7.7°(±0.2°), 11.3°(±0.2°), 16.8°(±0.2°), 18.3°(±0.2°), and 20.1°(±0.2°). In yet another alternative, as part of a twentieth embodiment, crystalline Form G is characterized by x-ray powder diffraction peaks at 2Θ angles selected from 5.7°(0.2°), 7.2°(±0.2°), 7.7°(±0.2°), 11.3°(±0.2°), 16.8°(±0.2°), 18.3°(±0.2°), and 20.1°(±0.2°). In another alternative, as part of a twentieth embodiment, crystalline Form G is characterized by an x-ray powder diffraction pattern comprising a peak at 2Θ angle 7.7°(±0.2°) and one or more peaks selected from 5.7°(±0.2°), 7.2°(±0.2°), 11.3°(±0.2°), 16.8°(±0.2°), 18.3°(±0.2°), and 20.1°(±0.2°). In yet another alternative, as part of a twentieth embodiment, crystalline Form G is characterized by an x-ray powder diffraction pattern comprising a peak at a 2Θ angle selected from 7.2°(±0.2°) and 7.7°(±0.2°), and one or more peaks selected from 5.7°(±0.2°), 11.3°(±0.2°), 16.8°(±0.2°), 18.3°(±0.2°), and 20.1°(±0.2°). In another alternative, as part of a twentieth embodiment, crystalline Form G is characterized by an x-ray powder diffraction pattern comprising a peak at a 2Θ angle selected from 7.2°(±0.2°), 7.7°(±0.2°), 18.3°(±0.2°), and one or more peaks selected from 5.7°(±0.2°), 11.3°(±0.2°), 16.8°(±0.2°), and 20.1°(±0.2°). In another alternative, as part of the twentieth embodiment, crystalline Form G is characterized by an x-ray powder diffraction pattern comprising a peak at a 2Θ angle selected from 7.2°(±0.2°), 7.7°(±0.2°), 16.8°(±0.2°), 18.3°(±0.2°), and one or more peaks selected from 5.7°(±0.2°), 11.3°(±0.2°), and 20.1°(±0.2°). In yet another alternative, as part of a twentieth embodiment, crystalline Form G is characterized by at least three, at least four, at least five, at least six, at least seven, at least eight or at least nine x-ray powder diffraction peaks at 2Θ angles selected from those in Table 20 (±0.2°). In yet another alternative, as part of a twentieth embodiment, crystalline Form G is characterized by x-ray powder diffraction peaks at 2Θ angles selected from those in Table 20 (±0.2°). In yet another alternative, as part of a twentieth embodiment, crystalline Form G is characterized by an x-ray powder diffraction pattern that is substantially the same as depicted in FIG. 44.

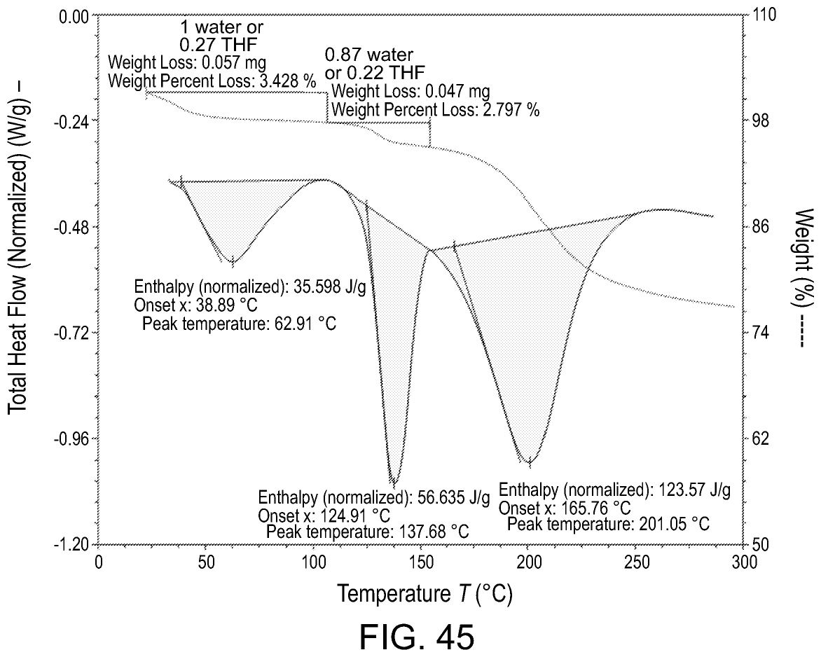

In a twenty-first embodiment, crystalline Form G, as described in the thirteenth or twentieth embodiments, is characterized by a differential scanning calorimetry (DSC) thermograph comprising endotherm peaks at 63° C.±5° C., 138° C.±5° C., and 202° C.±5° C. Alternatively, as part of a twenty-first embodiment, crystalline Form G, as described in the thirteenth or twentieth embodiment, is characterized by a differential scanning calorimetry (DSC) thermogram that is substantially similar to the one depicted in FIG. 45. In a twenty-second embodiment, crystalline Form G, as described in any one of the thirteenth, twentieth, or twenty-first embodiments, is characterized by a thermogravimetric analysis (TGA) thermogram comprising a weight loss of 3.428±0.01% up to 110° C.±2° C. and a weight loss of 2.797±0.01% up to 170° C.±2° C. Alternatively, as part of a twenty-second embodiment, crystalline Form G, as described in the thirteenth, twentieth, or twenty-first embodiments, is characterized by a thermogravimetric analysis (TGA) thermogram that is substantially similar to the one depicted in FIG. 45.

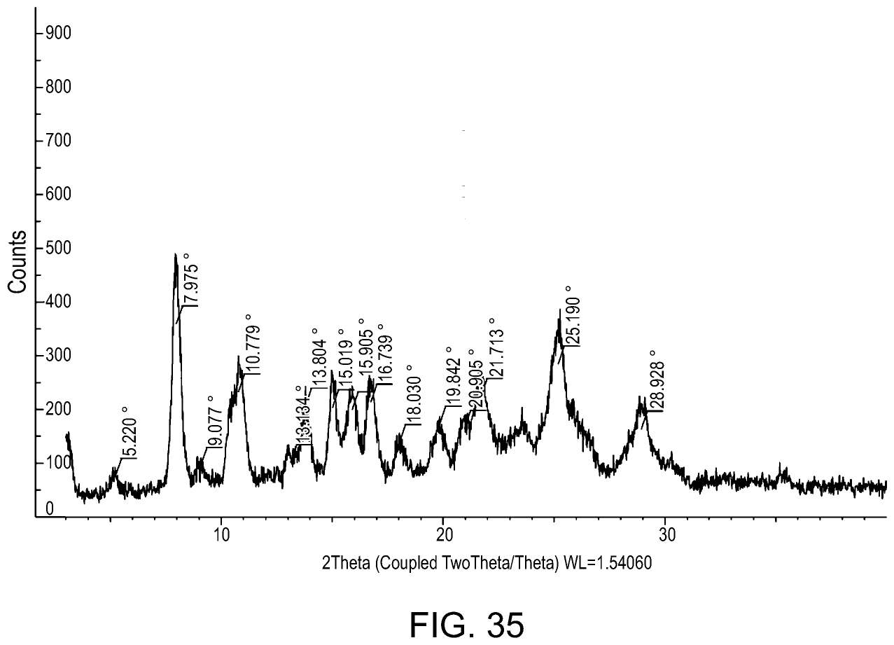

In a twenty-third embodiment, is provided a crystalline L-tartrate salt described herein as crystalline Form I. Alternatively, as part of a twenty-third embodiment, crystalline Form I is characterized by at least two x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°). In another alternative, as part of a twenty-third embodiment, crystalline Form I is characterized by at least three x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°). In another alternative, as part of a twenty-third embodiment, crystalline Form I is characterized by at least four x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°). In an alternative, as part of a twenty-third embodiment, crystalline Form I is characterized by at least five x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°). In another alternative, as part of a twenty-third embodiment, crystalline Form I is characterized by at least six x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°). In yet another alternative, as part of a twenty-third embodiment, crystalline Form I is characterized by x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°). In another alternative, as part of a twenty-third embodiment, crystalline Form I is characterized by an x-ray powder diffraction pattern comprising a peak at 2Θ angle 8.0°(±0.2°) and one or more peaks selected from 10.8°(±0.2°), 13.8°(±0.2°), 15.0°(±0.2°), 16.0°(±0.2°), 16.7°(±0.2°), and 25.2°(±0.2°). In yet another alternative, as part of a twenty-third embodiment, crystalline Form I is characterized by an x-ray powder diffraction pattern comprising a peak at a 2Θ angle selected from 8.0°(±0.2°) and 25.2°(±0.2°), and one or more peaks selected from 10.8°(±0.2°), 13.8°(±0.2°), 15.0°(±0.2°), 16.0°(±0.2°), and 16.7°(±0.2°). In another alternative, as part of a twenty-third embodiment, crystalline Form I is characterized by an x-ray powder diffraction pattern comprising a peak at a 2Θ angle selected from 8.0°(±0.2°), 25.2°(±0.2°), and 10.8°(±0.2°), and one or more peaks selected from 13.8°(±0.2°), 15.0°(±0.2°), 16.0°(±0.2°), and 16.7°(±0.2°). In another alternative, as part of the twenty-third embodiment, crystalline Form I is characterized by an x-ray powder diffraction pattern comprising a peak at a 2Θ angle selected from 8.0°(±0.2°), 25.2°(±0.2°), 10.8°(±0.2°), and 13.8°(±0.2°), and one or more peaks selected from 15.0°(±0.2°), 16.0°(±0.2°), and 16.7°(±0.2°). In yet another alternative, as part of a twenty-third embodiment, crystalline Form I is characterized by at least three, at least four, at least five, at least six, at least seven, at least eight or at least nine x-ray powder diffraction peaks at 2Θ angles selected from those in Table 17 (±0.2°). In yet another alternative, as part of a twenty-third embodiment, crystalline Form I is characterized by x-ray powder diffraction peaks at 20 angles selected from those in Table 17 (±0.2°). In yet another alternative, as part of a twenty-third embodiment, crystalline Form I is characterized by an x-ray powder diffraction pattern that is substantially the same as depicted in FIG. 35.

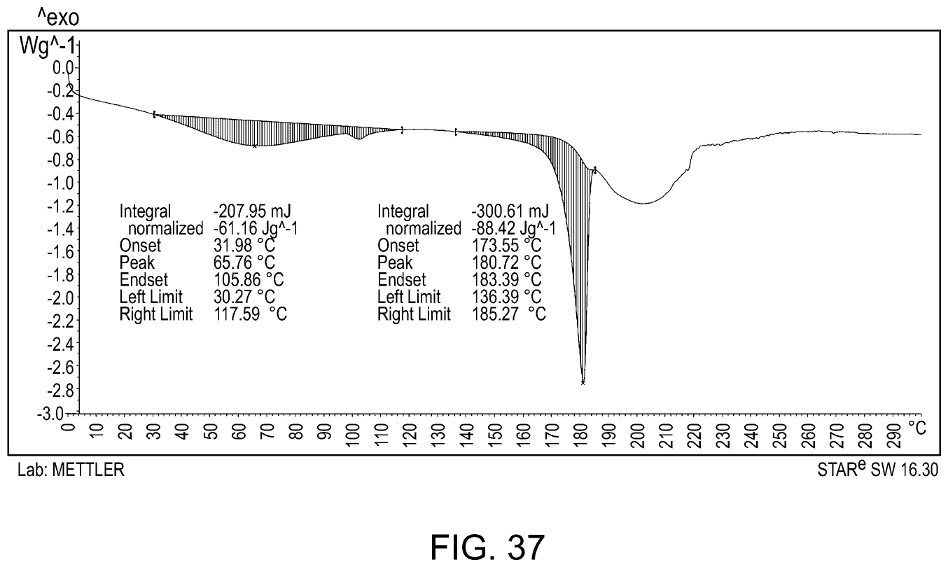

In a twenty-fourth embodiment, crystalline Form I, as described in the thirteenth or twenty-third embodiments, is characterized by a differential scanning calorimetry (DSC) thermograph comprising an endotherm peak at 65° C.±5° C. and 180° C.±5° C. Alternatively, as part of a twenty-fourth embodiment, crystalline Form I, as described in the thirteenth or twenty-third embodiment, is characterized by a differential scanning calorimetry (DSC) thermogram that is substantially similar to the one depicted in FIG. 37. In another alternative, as part of a twenty-fourth embodiment, crystalline Form I, as described in the thirteenth or twenty-third embodiment, is characterized by having a moisture content of about 0.05% to about 5% as measured by Karl-Fischer titration.

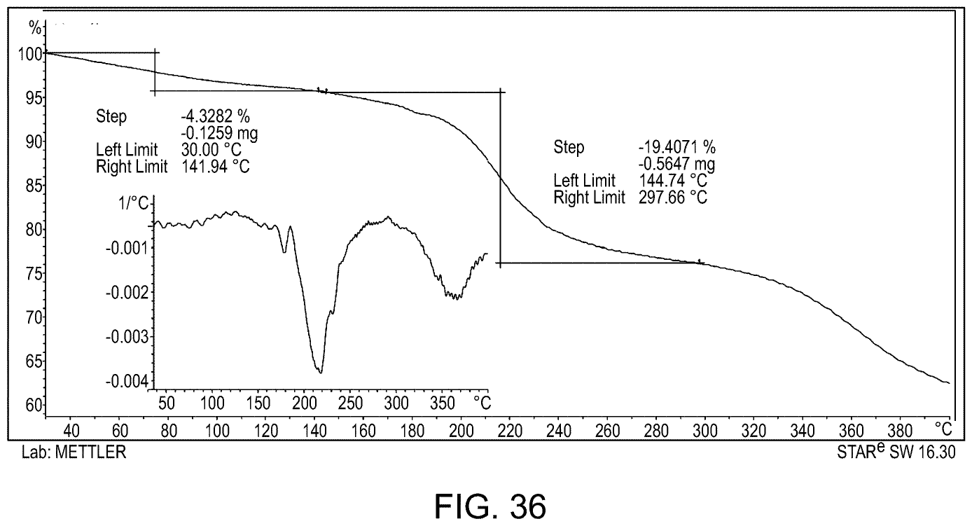

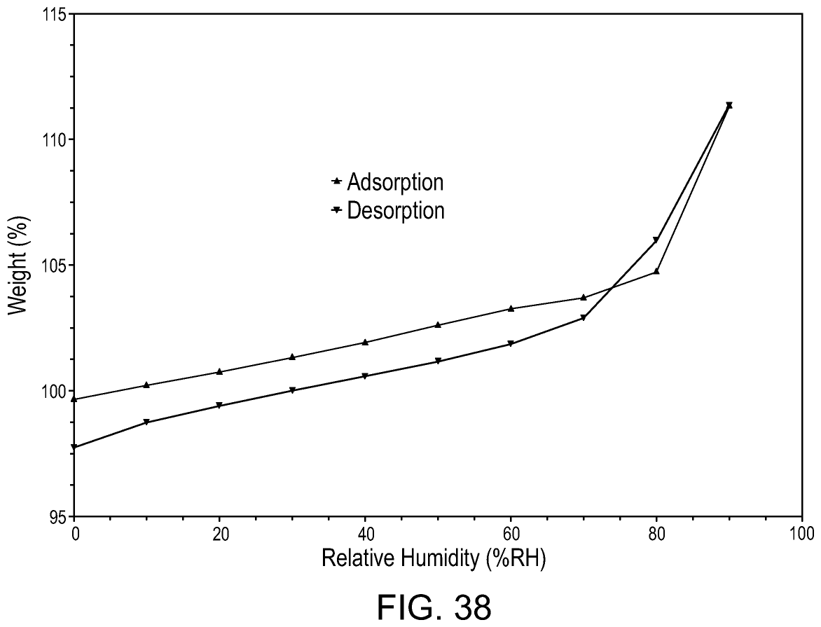

In a twenty-fifth embodiment, crystalline Form I, as described in any one of the thirteenth, twenty-third, or twenty-fourth embodiments, is characterized by a thermogravimetric analysis (TGA) thermogram comprising a weight loss of 4.0±0.5% up to 141° C.±2° C. and a weight loss of 19.5±0.5% up to 297° C.±2° C. Alternatively, as part of a twenty-fifth embodiment, crystalline Form I, as described in the thirteenth, twenty-third, or twenty-fourth embodiments, is characterized by a thermogravimetric analysis (TGA) thermogram that is substantially similar to the one depicted in FIG. 36. In another embodiment, crystalline Form I is characterized by a DVS that is substantially similar to the one depicted in FIG. 38.

In a twenty-sixth embodiment, is provided a crystalline form of a hydrochloride salt of Compound 1. Alternatively, as part of a twenty-sixth embodiment, is provided a crystalline hydrochloride salt of Compound 1 having the structural formula:

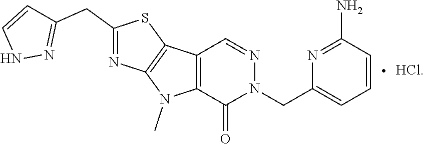

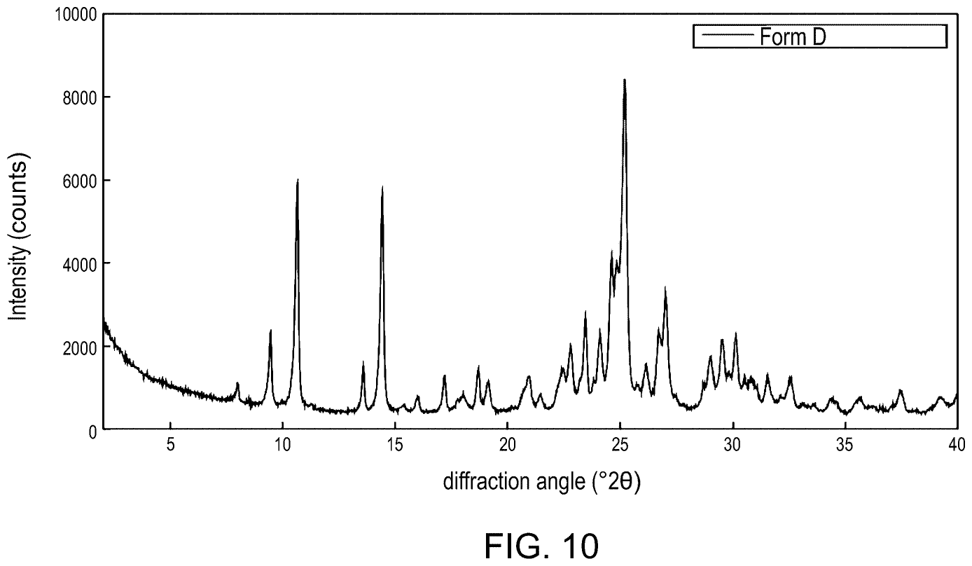

In a twenty-seventh embodiment, is provided a crystalline hydrochloride hydrochloride salt described herein as crystalline Form D. Alternatively, as part of a twenty-seventh embodiment, crystalline Form D is characterized by at least two x-ray powder diffraction peaks at 2Θ angles selected from 10.6°(±0.2°), 14.4°(±0.2°), 24.6(±0.2°), 24.8°(±0.2°), 25.2°(±0.2°), and 27.0°(±0.2°). In another alternative, as part of a twenty-seventh embodiment, crystalline Form D is characterized by at least three x-ray powder diffraction peaks at 2Θ angles selected from 10.6°(±0.2°), 14.4°(±0.2°), 24.6(±0.2°), 24.8°(±0.2°), 25.2°(±0.2°), and 27.0°(±0.2°). In another alternative, as part of a twenty-seventh embodiment, crystalline Form D is characterized by at least four x-ray powder diffraction peaks at 2Θ angles selected from 10.6°(±0.2°), 14.4°(±0.2°), 24.6(±0.2°), 24.8°(±0.2°), 25.2°(±0.2°), and 27.0°(±0.2°). In yet another alternative, as part of a twenty-seventh embodiment, crystalline Form D is characterized by at least five x-ray powder diffraction peaks at 2Θ angles selected from 10.6°(±0.2°), 14.4°(±0.2°), 24.6(±0.2°), 24.8°(±0.2°), 25.2°(±0.2°), and 27.0°(±0.2°). In another alternative, as part of a twenty-seventh embodiment, crystalline Form D is characterized by x-ray powder diffraction peaks at 2Θ angles selected from 10.6°(±0.2°), 14.4°(±0.2°), 24.6(±0.2°), 24.8°(±0.2°), 25.2°(±0.2°), and 27.0°(±0.2°). In yet another alternative, as part of a twenty-seventh embodiment, crystalline Form D is characterized by an x-ray powder diffraction pattern comprising a peak at 2Θ angle 25.2°(±0.2°) and one or more peaks selected from 10.6°(±0.2°), 14.4°(±0.2°), 24.6(±0.2°), 24.8°(±0.2°), and 27.0°(±0.2°). In another alternative, as part of a twenty-seventh embodiment, crystalline Form D is characterized by an x-ray powder diffraction pattern comprising a peak at a 2Θ angle selected from 25.2°(±0.2°) and 10.6°(±0.2°), and one or more peaks selected from 14.4°(±0.2°), 24.6(±0.2°), 24.8°(±0.2°), and 27.0°(±0.2°). In yet another alternative, as part of a twenty-seventh embodiment, crystalline Form D is characterized by an x-ray powder diffraction pattern comprising a peak at a 2Θ angle selected from 25.2°(±0.2°), 10.6°(±0.2°), and 14.4°(±0.2°), and one or more peaks selected from 24.6(±0.2°), 24.8°(±0.2°), and 27.0°(±0.2°). In yet another alternative, as part of a twenty-seventh embodiment, crystalline Form D is characterized by at least three, at least four, at least five, at least six, at least seven, at least eight or at least nine x-ray powder diffraction peaks at 2Θ angles selected from those in Table 13 (±0.2°). In yet another alternative, as part of a twenty-seventh embodiment, crystalline Form D is characterized by x-ray powder diffraction peaks at 2Θ angles selected from those in Table 13 (±0.2°). In a further alternative, as part of a twenty-seventh embodiment, crystalline Form D is characterized by an x-ray powder diffraction pattern that is substantially the same as depicted in FIG. 10.

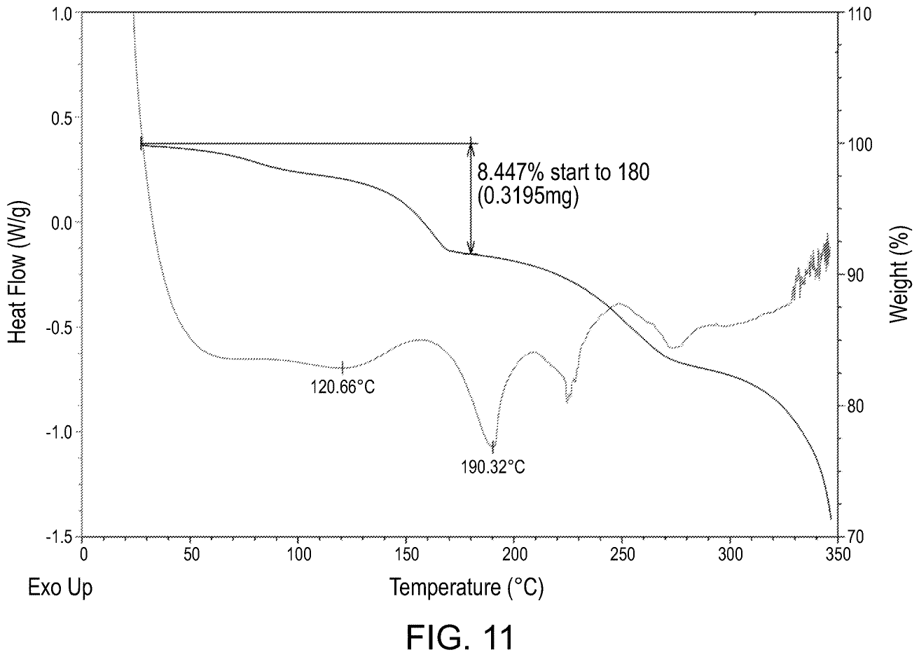

In a twenty-eighth embodiment, crystalline Form D, as described in the twenty-sixth or twenty-seventh embodiment, is characterized by a differential scanning calorimetry (DSC) thermograph comprising an endotherm peak at 190° C.±5° C. Alternatively, as part of a twenty-eighth t embodiment, crystalline Form D, as described in the twenty-sixth or twenty-seventh embodiments, is characterized by a differential scanning calorimetry (DSC) thermogram that is substantially similar to the one depicted in FIG. 11. In an alternative, as part of a twenty-eighth embodiment, crystalline Form D, as described in the twenty-sixth or twenty-seventh embodiment, is characterized by having a moisture content of about 1.0% to about 9% as measured by Karl-Fischer titration.

In a twenty-ninth embodiment, crystalline Form D, as described in any one of the twenty-sixth, twenty-seventh, or twenty-eighth embodiments, is characterized by a thermogravimetric analysis (TGA) thermogram comprising a weight loss of 8.5±0.5% up to 180° C.±2° C. Alternatively, as part of a twenty-ninth embodiment, crystalline Form D, as described in any one of the twenty-sixth, twenty-seventh, or twenty-eighth embodiments, is characterized by a thermogravimetric analysis (TGA) thermogram that is substantially similar to the one depicted in FIG. 11.

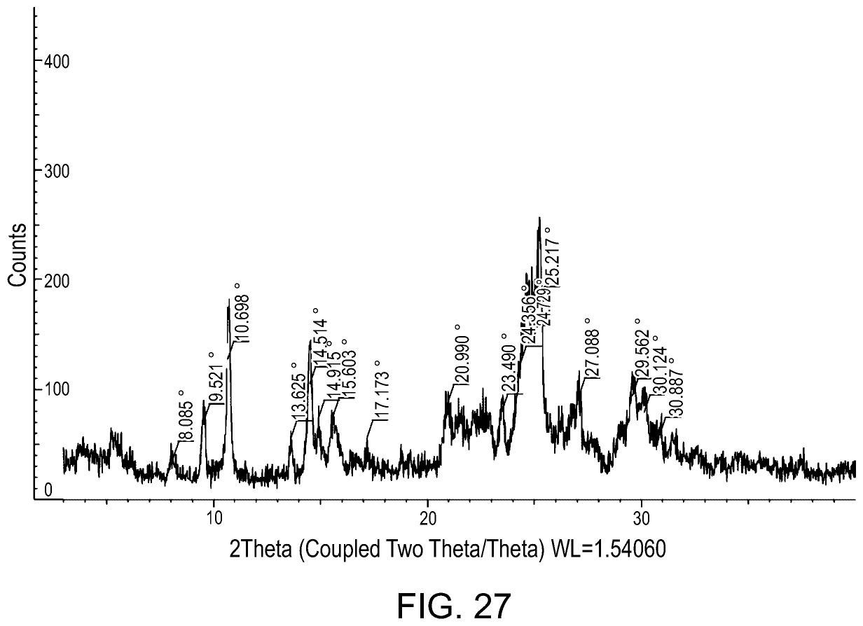

In a thirtieth embodiment, provided herein is an alternate crystalline hydrochloride salt of Form D, described herein as crystalline Form (D-1). Alternatively, as part of a thirtieth embodiment, crystalline Form D-1 is characterized by at least two x-ray powder diffraction peaks at 2Θ angles selected from 9.5°(±0.2°), 10.7°(±0.2°), 14.5°(±0.2°), 15.0°(±0.2°), 15.6° (±0.2°), 21.0°(±0.2°), 24.4°(±0.2°), and 25.2°(±0.2°). In another alternative, as part of a thirtieth embodiment, crystalline Form D-1 is characterized by at least three x-ray powder diffraction peaks at 2Θ angles selected from 9.5°(±0.2°), 10.7°(±0.2°), 14.5°(±0.2°), 15.0°(±0.2°), 15.6°(±0.2°), 21.0°(±0.2°), 24.4°(±0.2°), and 25.2°(±0.2°). In another alternative, as part of a thirtieth embodiment, crystalline Form D-1 is characterized by at least four x-ray powder diffraction peaks at 2Θ angles selected from 9.5°(±0.2°), 10.7°(±0.2°), 14.5°(±0.2°), 15.0°(±0.2°), 15.6°(±0.2°), 21.0°(±0.2°), 24.4°(±0.2°), and 25.2°(±0.2°). In yet another alternative, as part of a thirtieth embodiment, crystalline Form D-1 is characterized by at least five x-ray powder diffraction peaks at 2Θ angles selected from 9.5°(±0.2°), 10.7°(±0.2°), 14.5°(±0.2°), 15.0°(±0.2°), 15.6°(±0.2°), 21.0°(±0.2°), 24.4°(±0.2°), and 25.2°(±0.2°). In another alternative, as part of a thirtieth embodiment, crystalline Form D-1 is characterized by x-ray powder diffraction peaks at 2Θ angles selected from 9.5°(±0.2°), 10.7°(±0.2°), 14.5°(±0.2°), 15.0°(±0.2°), 15.6°(±0.2°), 21.0°(±0.2°), 24.4°(±0.2°), and 25.2°(±0.2°). In yet another alternative, as part of a thirtieth embodiment, crystalline Form D-1 is characterized by an x-ray powder diffraction pattern comprising a peak at 2Θ angle 25.2°(±0.2°) and one or more peaks selected from 9.5°(±0.2°), 10.7°(±0.2°), 14.5°(±0.2°), 15.0°(±0.2°), 15.6°(±0.2°), 21.0°(±0.2°), and 24.4°(±0.2°). In another alternative, as part of a thirtieth embodiment, crystalline Form D-1 is characterized by an x-ray powder diffraction pattern comprising a peak at 2Θ angles selected from 25.2°(±0.2°) and 10.7°(±0.2°), and one or more peaks selected from 9.5°(±0.2°), 14.5°(±0.2°), 15.0°(±0.2°), 15.6°(±0.2°), 21.0°(±0.2°), and 24.4°(±0.2°). In yet another alternative, as part of a thirtieth embodiment, crystalline Form D-1 is characterized by an x-ray powder diffraction pattern comprising a peak at 2Θ angles selected from 25.2°(±0.2°), 10.7°(±0.2°), and 14.5°(±0.2°), and one or more peaks selected from 9.5°(±0.2°), 15.0°(±0.2°), 15.6°(0.2°), 21.0°(±0.2°), and 24.4°(±0.2°). In yet another alternative, as part of a thirtieth embodiment, crystalline Form D-1 is characterized by at least three, at least four, at least five, at least six, at least seven, at least eight or at least nine x-ray powder diffraction peaks at 2Θ angles selected from those in Table 15 (±0.2°). In yet another alternative, as part of a thirtieth embodiment, crystalline Form D-1 is characterized by x-ray powder diffraction peaks at 2Θ angles selected from those in Table 15 (±0.2°). In a further alternative, as part of the thirtieth embodiment, crystalline Form D-1 is characterized by an x-ray powder diffraction pattern that is substantially the same as depicted in FIG. 27.

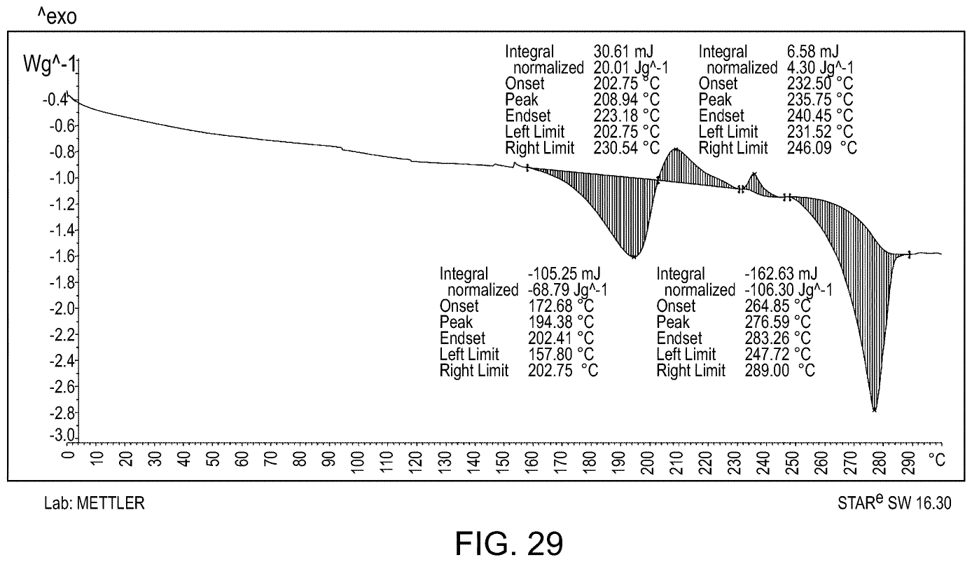

In a thirty-first embodiment, crystalline Form D-1, as described in the twenty-sixth or thirtieth embodiment, is characterized by a differential scanning calorimetry (DSC) thermograph comprising an endotherm peak at 194° C.±5° C., an exotherm peak at 208° C.±5° C., an exotherm peak at 235° C.±5° C., and an endotherm peak at 276° C.±5° C. Alternatively, as part of a thirty-first embodiment, crystalline Form D-1, as described in the twenty-sixth or thirtieth embodiments, is characterized by a differential scanning calorimetry (DSC) thermogram that is substantially similar to the one depicted in FIG. 29.

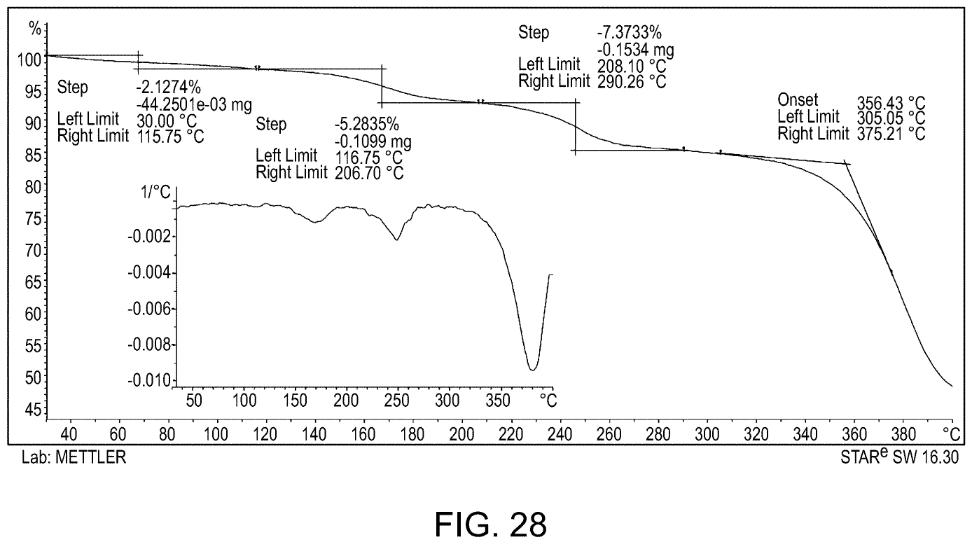

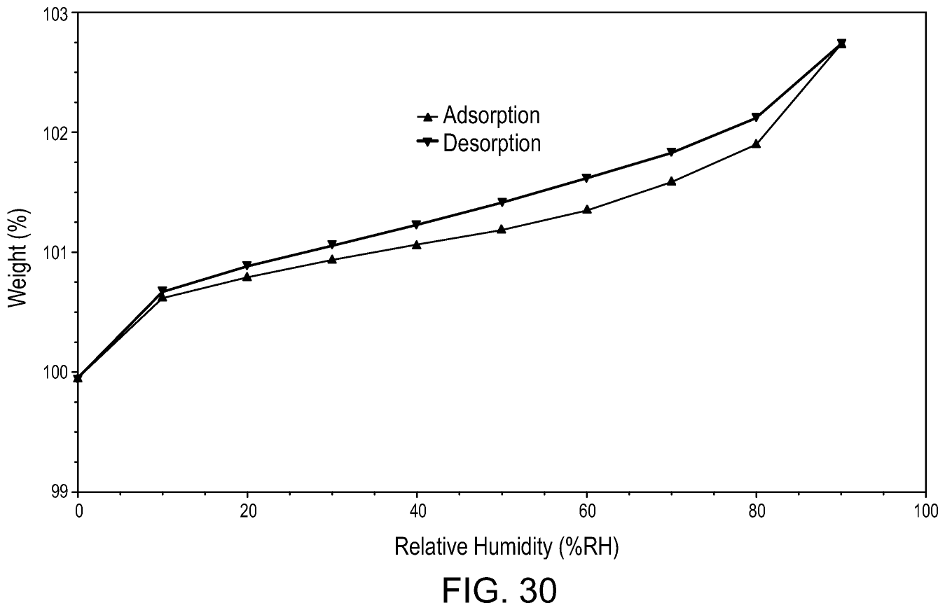

In a thirty-second embodiment, crystalline Form D-1, as described in any one of the twenty-sixth, thirtieth, or thirty-first embodiments, is characterized by a thermogravimetric analysis (TGA) thermogram comprising a weight loss of 5.3±0.5% up to 206° C.±2° C. and a weight loss of 7.4±0.5% up to 290° C.±2° C. Alternatively, as part of a thirty-second embodiment, crystalline Form D-1, as described in any one of the twenty-sixth, thirtieth, or thirty-first embodiments, is characterized by a thermogravimetric analysis (TGA) thermogram that is substantially similar to the one depicted in FIG. 28. As part of a thirty-second embodiment, crystalline Form D-1, as described in any one of the twenty-sixth thirtieth, or thirty-first embodiments, is characterized by a DVS that is substantially similar to the one depicted in FIG. 30.

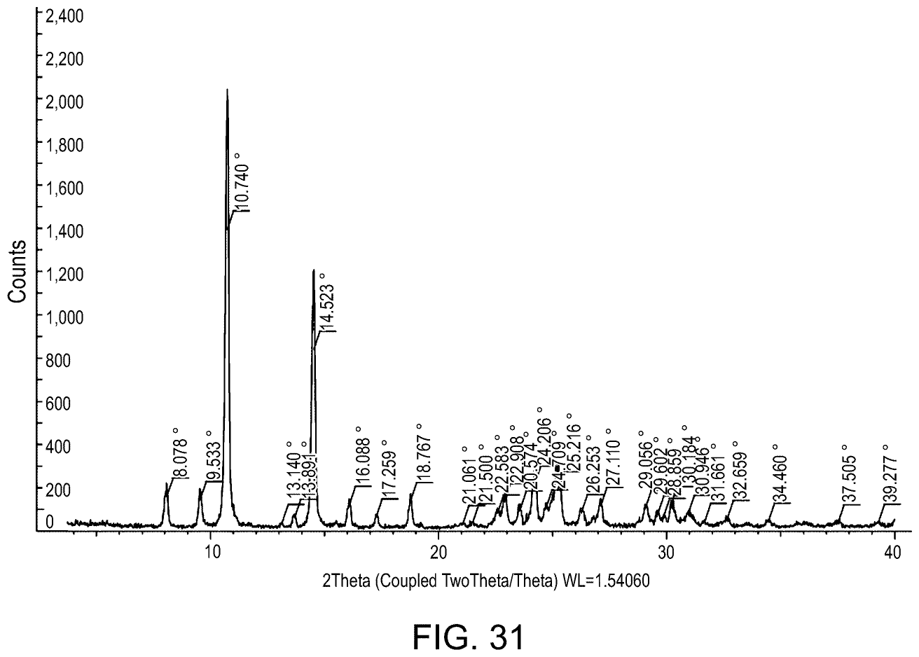

In a thirty-third embodiment, provided herein is a further alternate crystalline hydrochloride salt of Form D described herein as crystalline Form (D-2). Alternatively, as part of a thirty-third embodiment, crystalline Form (D-2) is characterized by at least two x-ray powder diffraction peaks at 2Θ angles selected from 8.1°(±0.2°), 9.5°(±0.2°), 10.7°(±0.2°), 14.5°(±0.2°), 24.2°(±0.2°), and 25.2°(±0.2°). In another alternative, as part of a thirty-third embodiment, crystalline Form (D-2) is characterized by at least three x-ray powder diffraction peaks at 2Θ angles selected from 8.1°(±0.2°), 9.5°(±0.2°), 10.7°(±0.2°), 14.5°(±0.2°), 24.2°(±0.2°), and 25.2°(±0.2°). In another alternative, as part of a thirty-third embodiment, crystalline Form (D-2) is characterized by at least four x-ray powder diffraction peaks at 2Θ angles selected from 8.1°(±0.2°), 9.5°(±0.2°), 10.7°(±0.2°), 14.5°(±0.2°), 24.2° (±0.2°), and 25.2°(±0.2°). In an alternative, as part of a thirty-third embodiment, crystalline Form (D-2) is characterized by at least five x-ray powder diffraction peaks at 2Θ angles selected from 8.1°(±0.2°), 9.5°(±0.2°), 10.7°(±0.2°), 14.5°(±0.2°), 24.2°(±0.2°), and 25.2°(±0.2°). In another alternative, as part of a thirty-third embodiment, crystalline Form (D-2) is characterized by at least six x-ray powder diffraction peaks at 2Θ angles selected from 8.1°(±0.20), 9.50(±0.20), 10.7°(±0.20), 14.5°(±0.20), 24.2°(±0.20), and 25.2°(±0.20). In yet another alternative, as part of a thirty-third embodiment, crystalline Form (D-2) is characterized by x-ray powder diffraction peaks at 2Θ angles selected from 8.1°(±0.2°), 9.5°(±0.2°), 10.7°(±0.2°), 14.5°(±0.2°), 24.2°(±0.2°), and 25.2°(±0.2°). In another alternative, as part of a thirty-third embodiment, crystalline Form (D-2) is characterized by an x-ray powder diffraction pattern comprising a peak at 2Θ angle 10.7°(±0.2°) and one or more peaks selected from 8.1°(±0.2°), 9.5°(±0.2°), 14.5°(±0.2°), 24.2°(±0.2°), and 25.2°(±0.2°). In yet another alternative, as part of a thirty-third embodiment, crystalline Form (D-2) is characterized by an x-ray powder diffraction pattern comprising a peak at a 2Θ angle selected from 10.7°(±0.2°) and 14.5°(±0.2°), and one or more peaks selected from 8.1°(±0.2°), 9.5°(±0.2°), 24.2°(±0.2°), and 25.2°(±0.2°). In another alternative, as part of a thirty-third embodiment, crystalline Form (D-2) is characterized by an x-ray powder diffraction pattern comprising a peak at a 2Θ angle selected from 10.7°(±0.2°), 14.5°(±0.2°), and 24.2°(±0.2°), and one or more peaks selected from 8.1°(±0.2°), 9.5°(±0.2°), and 25.2°(±0.2°). In another alternative, as part of the thirty-third embodiment, crystalline Form (D-2) is characterized by an x-ray powder diffraction pattern comprising a peak at a 2Θ angle selected from 10.7°(±0.2°), 14.5°(±0.2°), 24.2°(±0.2°), and 25,2°(±0.2°), and one or more peaks selected from 8.1°(±0.2°) and 9.5°(±0.2°). In yet another alternative, as part of a thirty-third embodiment, crystalline Form (D-2) is characterized by at least three, at least four, at least five, at least six, at least seven, at least eight or at least nine x-ray powder diffraction peaks at 2Θ angles selected from those in Table 16 (±0.2°). In yet another alternative, as part of a thirty-third embodiment, crystalline Form (D-2) is characterized by x-ray powder diffraction peaks at 2Θ angles selected from those in Table 16 (±0.2°). In yet another alternative, as part of a thirty-third embodiment, crystalline Form (D-2) is characterized by an x-ray powder diffraction pattern that is substantially the same as depicted in FIG. 31.

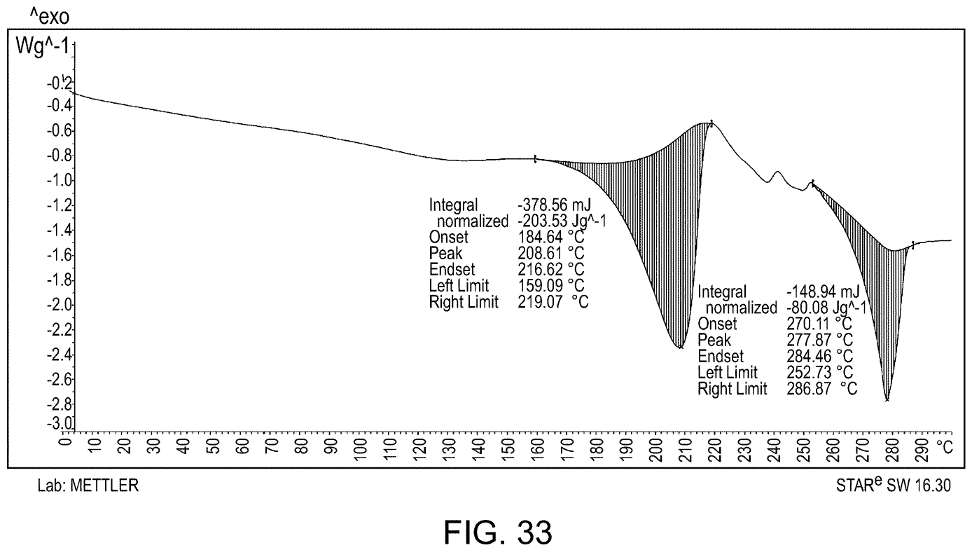

In a thirty-fourth embodiment, crystalline Form (D-2), as described in the twenty-sixth or thirty-third embodiment, is characterized by a differential scanning calorimetry (DSC) thermograph comprising an endotherm peak at 208° C.±5° C. and 277° C.±5° C. Alternatively, as part of a thirty-fourth embodiment, crystalline Form (D-2), as described in the twenty-sixth or thirty-third embodiment, is characterized by a differential scanning calorimetry (DSC) thermogram that is substantially similar to the one depicted in FIG. 33.

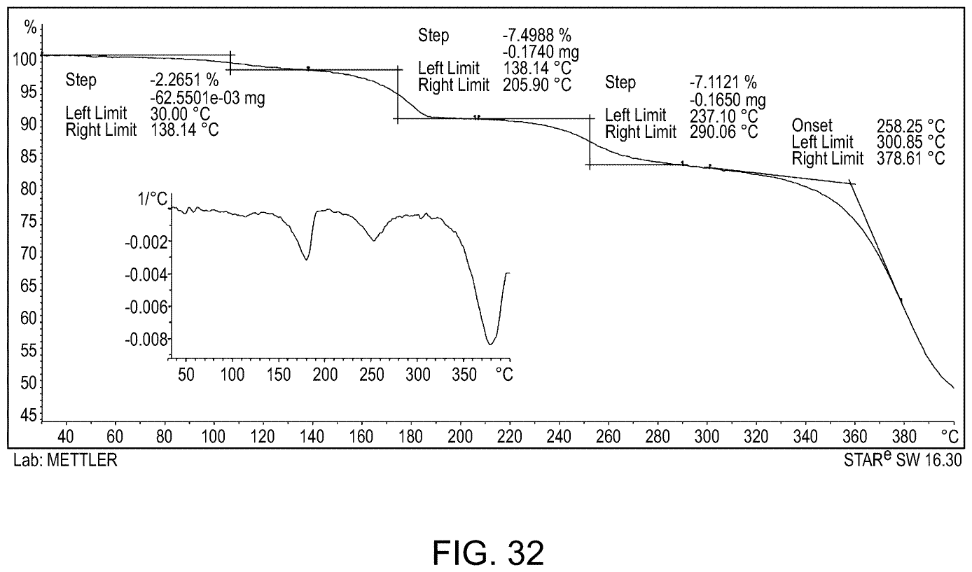

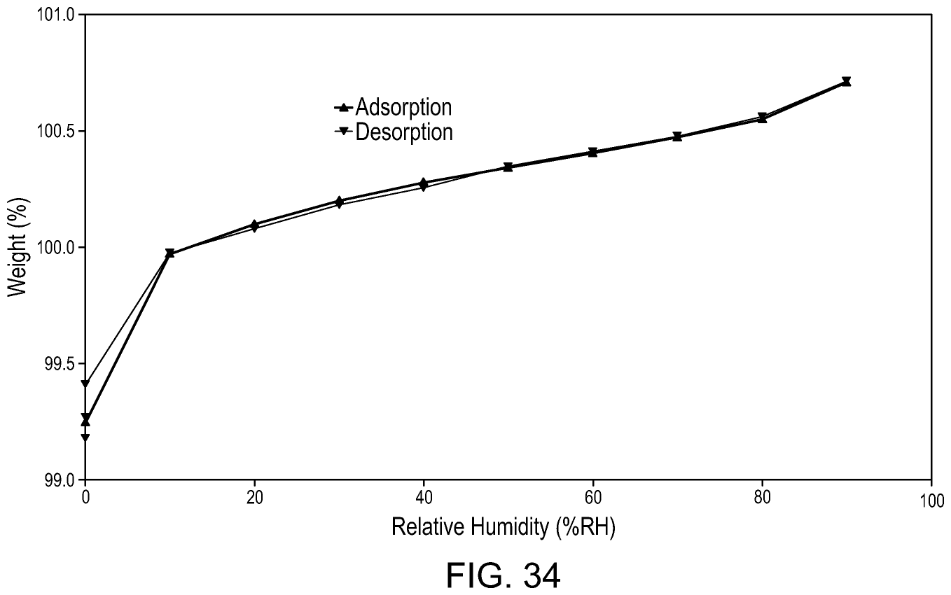

In a thirty-fifth embodiment, crystalline Form (D-2), as described in any one of the twenty-sixth, thirty-third or thirty-fourth embodiments, is characterized by a thermogravimetric analysis (TGA) thermogram comprising a weight loss of 7.5±0.5% up to 205° C.±2° C. and a weight loss of 7.1±0.5% up to 290° C.±2° C. Alternatively, as part of a thirty-fifth embodiment, crystalline Form (D-2), as described in the twenty-sixth, thirty-third, or thirty-fourth embodiments, is characterized by a thermogravimetric analysis (TGA) thermogram that is substantially similar to the one depicted in FIG. 32. In another embodiment, crystalline Form (D-2) is characterized by a DVS that is substantially similar to the one depicted in FIG. 34. In another alternative of a thirty-fifth embodiment, crystalline Form (D-2), as described in the twenty-sixth, thirty-third, or thirty-fourth embodiment, is characterized by having a moisture content of about 5% to about 10% as measured by Karl-Fischer titration.

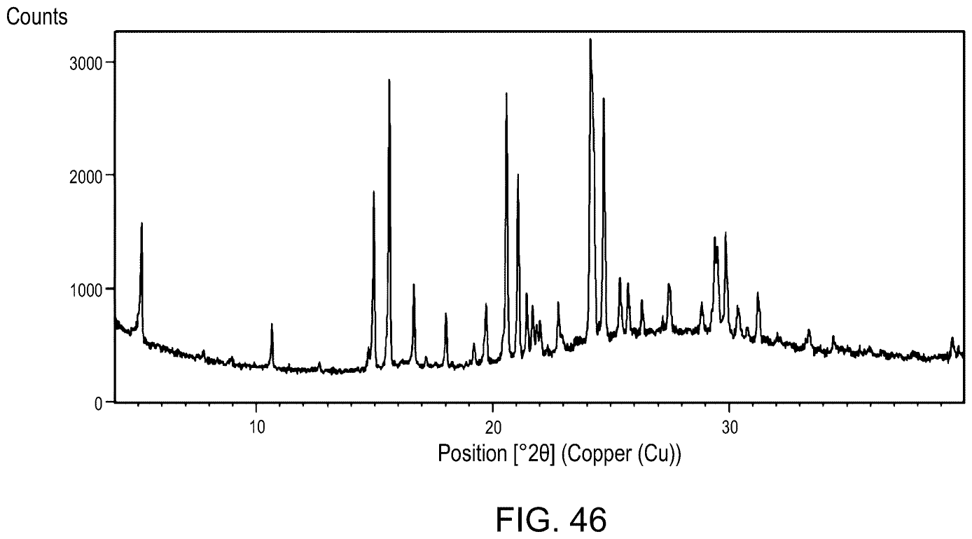

In a thirty-sixth embodiment, is provided a hydrochloride salt described herein as crystalline Form J. Alternatively, as part of a thirty-sixth embodiment, crystalline Form J is characterized by at least two x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°). In another alternative, as part of a thirty-sixth embodiment, crystalline Form J is characterized by at least three x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°). In another alternative, as part of a thirty-sixth embodiment, crystalline Form J is characterized by at least four x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°). In yet another alternative, as part of a thirty-sixth embodiment, crystalline Form J is characterized by at least five x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°). In another alternative, as part of a thirty-sixth embodiment, crystalline Form J is characterized by x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°). In yet another alternative, as part of a thirty-sixth embodiment, crystalline Form J is characterized by an x-ray powder diffraction pattern comprising a peak at 2Θ angle 24.1°(±0.2°), and one or more peaks selected from 15.0°(±0.2°), 15.6°(±0.2°), 20.4(±0.2°), 20.6°(±0.2°), 21.1°(±0.2°), and 24.7°(±0.2°). In another alternative, as part of a thirty-sixth embodiment, crystalline Form J is characterized by an x-ray powder diffraction pattern comprising a peak at a 2Θ angle selected from 15.6°(±0.2°) and 24.1°(±0.2°), and one or more peaks selected from 15.0°(±0.2°), 20.4(±0.2°), 20.6°(±0.2°), 21.1°(±0.2°), and 24.7°(±0.2°). In yet another alternative, as part of a thirty-sixth embodiment, crystalline Form J is characterized by an x-ray powder diffraction pattern comprising a peak at a 2Θ angle selected from 15.6°(±0.2°), 20.6°(±0.2°), and 24.1°(±0.2°), and one or more peaks selected from 15.0°(±0.2°), 20.4(±0.2°), 21.1°(±0.2°), and 24.7°(±0.2°). In yet another alternative, as part of a thirty-sixth embodiment, crystalline Form J is characterized by at least three, at least four, at least five, at least six, at least seven, at least eight or at least nine x-ray powder diffraction peaks at 2Θ angles selected from those in Table 21 (±0.2°). In yet another alternative, as part of a thirty-sixth embodiment, crystalline Form J is characterized by x-ray powder diffraction peaks at 2Θ angles selected from those in Table 21 (±0.2°). In a further alternative, as part of a thirty-sixth embodiment, crystalline Form J is characterized by an x-ray powder diffraction pattern that is substantially the same as depicted in FIG. 46.

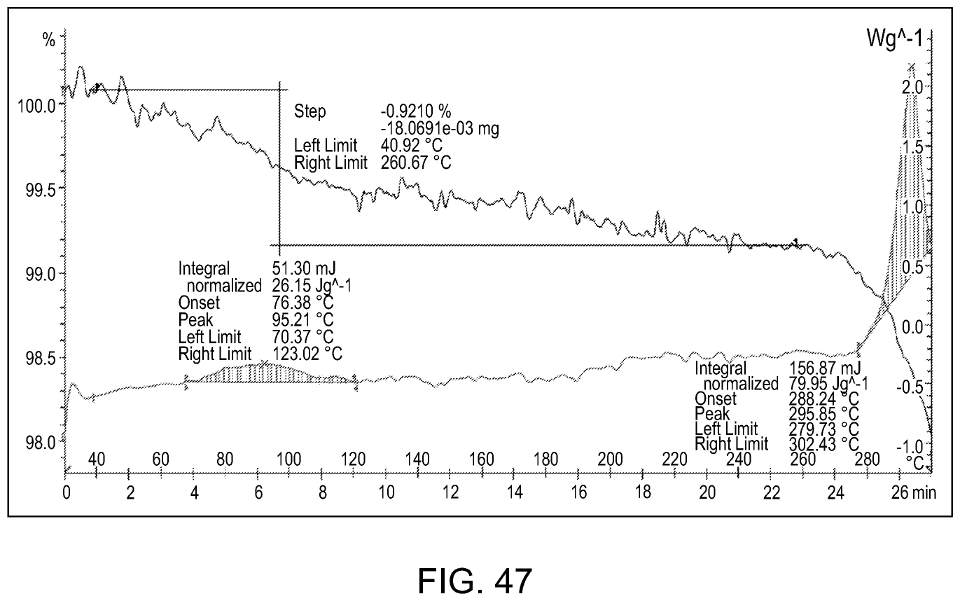

In a thirty-seventh embodiment, crystalline Form J, is described in the twenty-sixth or thirty-sixth embodiment, is characterized by a differential scanning calorimetry (DSC) thermograph comprising an endotherm peak at 76° C.±5° C. and 229° C.±5° C. Alternatively, as part of a thirty-seventh embodiment, crystalline Form J, as described in the twenty-sixth or thirty-sixth embodiment, is characterized by a differential scanning calorimetry (DSC) thermogram that is substantially similar to the one depicted in FIG. 47.

In a thirty-eighth embodiment, crystalline Form J, as described in any one of the twenty-sixth, thirty-sixth, or thirty-seventh embodiments, is characterized by a thermogravimetric analysis (TGA) thermogram comprising a weight loss of 0.92±0.05% up to 250° C.±2° C. Alternatively, as part of a thirty-eighth embodiment, crystalline Form J, as described in the twenty-sixth, thirty-sixth, or thirty-seventh embodiments, is characterized by a thermogravimetric analysis (TGA) thermogram that is substantially similar to the one depicted in FIG. 47.

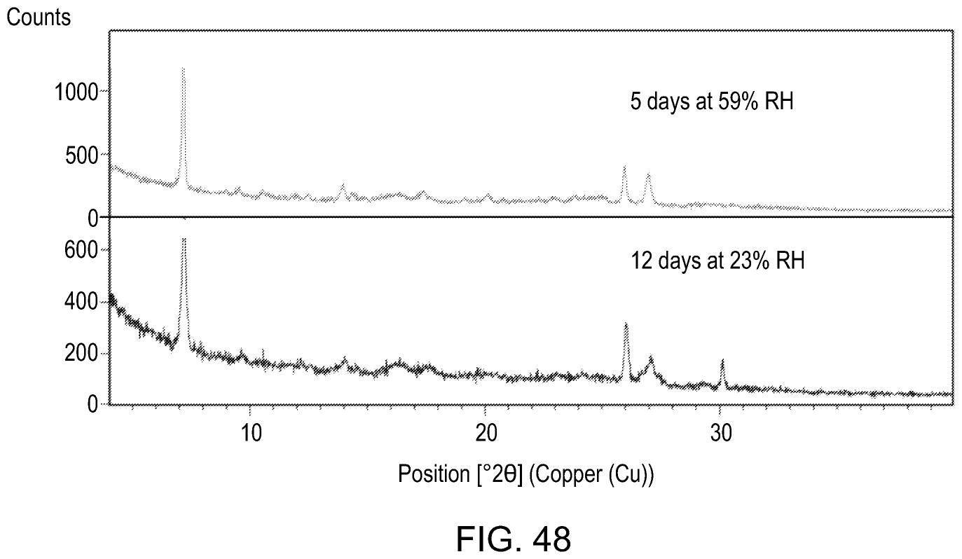

In a thirty-ninth embodiment, is provided a hydrochloride salt described herein as crystalline Form K. Alternatively, as part of a thirty-ninth embodiment, crystalline Form K is characterized by at least two x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°). In another alternative, as part of a thirty-ninth embodiment, crystalline Form K is characterized by at least three x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°). In another alternative, as part of a thirty-ninth embodiment, crystalline Form K is characterized by at least four x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°). In yet another alternative, as part of a thirty-ninth embodiment, crystalline Form K is characterized by at least five x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°). In another alternative, as part of a thirty-ninth embodiment, crystalline Form K is characterized by x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°). In yet another alternative, as part of a thirty-ninth embodiment, crystalline Form K is characterized by an x-ray powder diffraction pattern comprising a peak at 2Θ angle 7.2°(±0.2°), and one or more peaks selected from 9.5°(±0.2°), 10.6(±0.2°), 14.0°(±0.2°), 17.4°(±0.2°), 26.0°(±0.2°), and 27.0°(±0.2°). In another alternative, as part of a thirty-ninth embodiment, crystalline Form K is characterized by an x-ray powder diffraction pattern comprising a peak at a 2Θ angle selected from 7.2°(±0.2°), and 26.0°(±0.2°), and one or more peaks selected from 9.5°(±0.2°), 10.6(±0.2°), 14.0°(±0.2°), 17.4°(±0.2°), and 27.0°(±0.2°). In yet another alternative, as part of a thirty-ninth embodiment, crystalline Form K is characterized by an x-ray powder diffraction pattern comprising a peak at a 2Θ angle selected from 7.2°(±0.2°), 26.0°(±0.2°), and 27.0°(±0.2°), and one or more peaks selected from 9.5°(±0.2°), 10.6(±0.2°), 14.0°(±0.2°), and 17.4°(±0.2°). In yet another alternative, as part of a thirty-ninth embodiment, crystalline Form K is characterized by at least three, at least four, at least five, at least six, at least seven, at least eight or at least nine x-ray powder diffraction peaks at 2Θ angles selected from those in Table 22 (±0.2°). In yet another alternative, as part of a thirty-ninth embodiment, crystalline Form K is characterized by x-ray powder diffraction peaks at 2Θ angles selected from those in Table 22 (±0.2°). In a further alternative, as part of a thirty-ninth embodiment, crystalline Form K is characterized by an x-ray powder diffraction pattern that is substantially the same as depicted in FIG. 48.

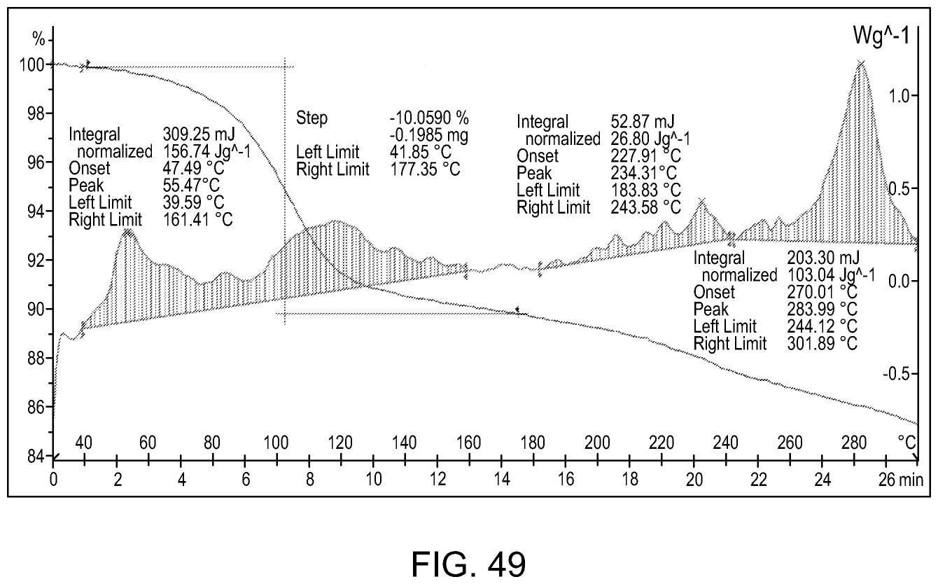

In a fortieth embodiment, crystalline Form K, is described in the twenty-sixth and thirty-ninth embodiment, is characterized by a differential scanning calorimetry (DSC) thermograph comprising exotherm peaks at 55° C.±5° C., 234° C.±5° C., and 284° C.±5° C. Alternatively, as part of a fortieth embodiment, crystalline Form K, as described in the twenty-sixth and thirty-ninth embodiment, is characterized by a differential scanning calorimetry (DSC) thermogram that is substantially similar to the one depicted in FIG. 49.

In a forty-first embodiment, crystalline Form K, as described in any one of the twenty-sixth, thirty-ninth, and fortieth embodiments, is characterized by a thermogravimetric analysis (TGA) thermogram comprising a weight loss of 10.06±0.05% up to 180° C.±2° C. Alternatively, as part of a forty-first embodiment, crystalline Form K, as described in the twenty-sixth, thirty-ninth, and fortieth embodiments, is characterized by a thermogravimetric analysis (TGA) thermogram that is substantially similar to the one depicted in FIG. 49.

In a forty-second embodiment, provided is a crystalline free base (non-salt) of Compound 1. Alternatively, as part of a forty-second embodiment, provided herein is a crystalline free base monohydrate of a compound having the structural formula:

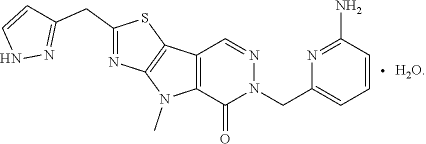

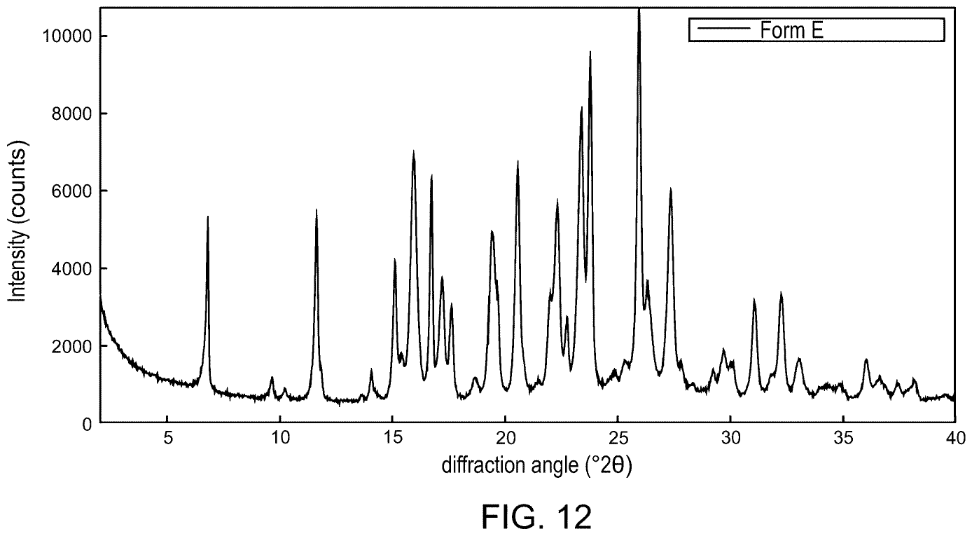

In a forty-third embodiment, the crystalline free base monohydrate of Compound 1 is described herein as crystalline Form E. Alternatively, as part of a forty-third embodiment, crystalline Form E is characterized by at least two x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°). In another alternative, as part of a forty-third embodiment, crystalline Form E is characterized by at least three x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°). In another alternative, as part of a forty-third embodiment, crystalline Form E is characterized by at least four x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°). In an alternative, as part of a forty-third embodiment, crystalline Form E is characterized by at least five x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°). In another alternative, as part of a forty-third embodiment, crystalline Form E is characterized by at least six x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°). In yet another alternative, as part of a forty-third embodiment, crystalline Form E is characterized by x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°). In another alternative, as part of a forty-third embodiment, crystalline Form E is characterized by an x-ray powder diffraction pattern comprising a peak at 2Θ angle 26.0°(±0.2°) and one or more peaks selected from 11.6°(±0.2°), 16.0°(±0.2°), 16.8°(±0.2°), 20.6°(±0.2°), 23.8°(±0.2°), and 27.4°(±0.2°). In yet another alternative, as part of a forty-third embodiment, crystalline Form E is characterized by an x-ray powder diffraction pattern comprising a peak at a 2Θ angle selected from 26.0°(±0.2°) and 23.8°(±0.2°), and one or more peaks selected from 11.6°(±0.2°), 16.0°(±0.2°), 16.8°(±0.2°), 20.6°(±0.2°), and 27.4°(±0.2°). In another alternative, as part of a forty-third embodiment, crystalline Form E is characterized by an x-ray powder diffraction pattern comprising a peak at a 2Θ angle selected from 26.0°(±0.2°), 23.8°(±0.2°), and 16.0°(±0.2°), and one or more peaks selected from 11.6°(±0.2°), 16.8°(±0.2°), 20.6°(±0.2°), and 27.4°(±0.2°). In another alternative, as part of the forty-third embodiment, crystalline Form E is characterized by an x-ray powder diffraction pattern comprising a peak at a 2Θ angle selected from 26.0°(±0.2°), 23.8°(±0.2°), 16.0°(±0.2°), and 20.6°(±0.2°), and one or more peaks selected from 11.6°(±0.2°), 16.8°(±0.2°), and 27.4°(±0.2°). In yet another alternative, as part of a forty-third embodiment, crystalline Form E is characterized by at least three, at least four, at least five, at least six, at least seven, at least eight or at least nine x-ray powder diffraction peaks at 2Θ angles selected from those in Table 14 (±0.2°). In yet another alternative, as part of a forty-third embodiment, crystalline Form E is characterized by x-ray powder diffraction peaks at 2Θ angles selected from those in Table 14 (±0.2°). In yet another alternative, as part of a forty-third embodiment, crystalline Form E is characterized by an x-ray powder diffraction pattern that is substantially the same as depicted in FIG. 12.

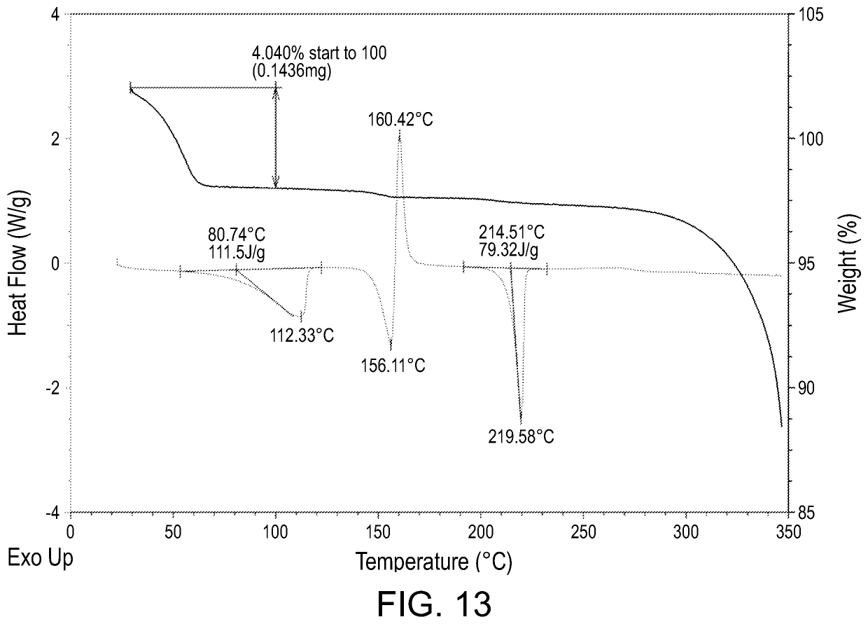

In a forty-fourth embodiment, crystalline Form E, as described in the forty-second-fourth or forty-third embodiments, is characterized by a differential scanning calorimetry (DSC) thermograph comprising an endotherm peak at 112° C.±5° C., 156° C.±5° C., and 219° C.±5° C., and an exotherm peak at 160° C.±5° C. Alternatively, as part of a forty-fourth embodiment, crystalline Form E, as described in the forty-second or forty-third embodiments, is characterized by a differential scanning calorimetry (DSC) thermogram that is substantially similar to the one depicted in FIG. 13.

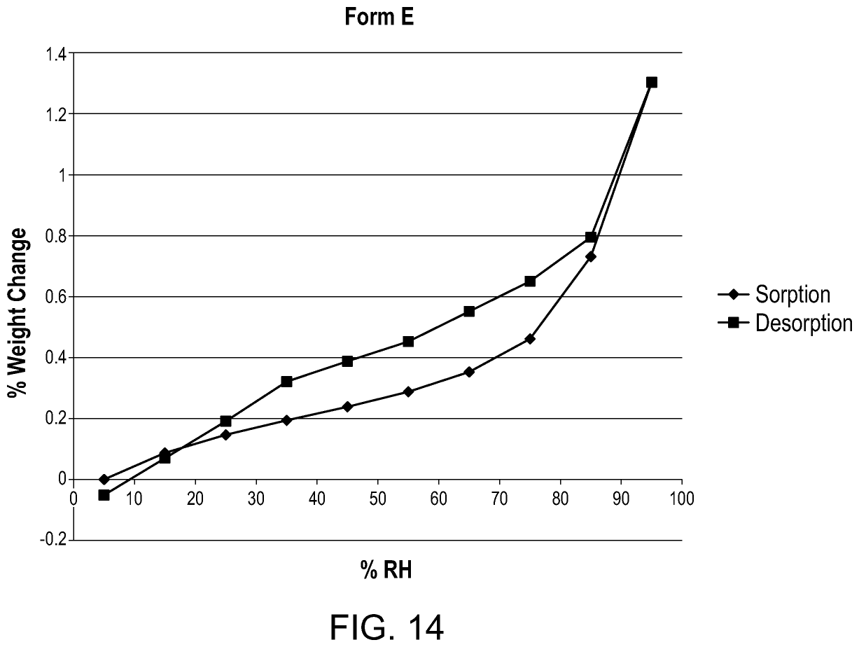

In a forty-fifth embodiment, crystalline Form E, as described in any one of the forty-second, forty-third, or forty-fourth embodiments, is characterized by a thermogravimetric analysis (TGA) thermogram comprising a weight loss of 4.0±0.5% up to 100° C.±2° C. Alternatively, as part of a forty-fifth embodiment, crystalline Form E, as described in any one of the forty-second, forty-third, or forty-fourth embodiments, is characterized by a thermogravimetric analysis (TGA) thermogram that is substantially similar to the one depicted in FIG. 13. In another embodiment, crystalline Form E is characterized by a DVS that is substantially similar to the one depicted in FIG. 14.

In one embodiment, the crystalline forms described herein (e.g., in any one of the first to forty-fifth embodiments) have a chemical purity of at least 60%, at least 70%, at least 80%, at least 90%, at least 95%, or at least 99% by weight as determined by HPLC.

In a forty-sixth embodiment, provided are spray dried dispersions that are prepared using a crystalline form as described herein (e.g., such as any one of those in the first to forty-fourth embodiments), comprising dissolving a crystalline form as described herein (e.g., such as any one of those in the first to forty-fourth embodiments), and a dispersion polymer in a solvent to form a feed solution; and removing the solvent to form the spray dried dispersion.

Also provided herein, as part of a forty-sixth embodiment, are spray dried dispersions that are prepared using at least one crystalline form described herein (e.g., such as any one of those in the first to forty-fifth embodiments) and that such resultant spray dried dispersions comprise Compound 1 that is substantially amorphous. Alternatively, provided herein, as part of a forty-sixth embodiment, are spray dried dispersions prepared using at least one crystalline form described herein (e.g., such as any one of those in the first to forty-fifth embodiments), wherein the resultant spray dried dispersions are formulated in a tablet and comprise Compound 1 that is substantially amorphous.

In a forty-seventh embodiment, the spray dried dispersions are prepared using the crystalline hemi-hydrate hemi-sulfate salt Form A (e.g., those described in any one of the first, second, third, fourth, fifth, sixth, seventh, or eighth embodiments) and at least one polymer. Alternatively, the spray dried dispersions are prepared using the crystalline hemi-hydrate hemi-sulfate salt Form A, the crystalline phosphate salt Form B, the crystalline DL-tartrate salt form C, the crystalline hydrochloride salt Form D, or the crystalline free base monohydrate Form E, and at least one polymer.

In another alternative, as part of the forty-seventh embodiment, the spray dried dispersions are prepared using the crystalline hemi-hydrate hemi-sulfate salt Form H, the crystalline DL-tartrate salt Form C-1, the crystalline DL-tartrate salt Form F, the crystalline DL-tartrate salt Form G, the crystalline hydrochloride salt Form D (i.e., Forms D, D-1, and D-2), the crystalline hydrochloride salt Form J, the crystalline hydrochloride salt Form K, or the crystalline L-tartrate salt Form I, and at least one polymer.

In a forty-eighth embodiment, tablets comprising the spray dried dispersions, comprise about 55% by weight, about 50% by weight, about 45% by weight, about 40% by weight, about 35%, by weight, about 30% by weight, about 25% by weight, or about 20% by weight of Compound 1. In one aspect the tablets comprising a spray dried dispersion are prepared using at least one of the crystalline forms described herein. In another aspect the tablets comprising a spray dried dispersion are prepared using crystalline hemi-hydrate hemi-sulfate salt Form A. In another aspect the tablets comprising the spray dried dispersion are prepared using crystalline phosphate salt Form B. In another aspect the tablets comprising the spray dried dispersion are prepared using the crystalline hemi-hydrate hemi-sulfate salt Form A, the crystalline phosphate salt Form B, the crystalline DL-tartrate salt form C, the crystalline hydrochloride salt Form D, or the crystalline free base monohydrate Form E.

In another alternative, as part of the forty-eighth embodiment, the tablets comprising the spray dried dispersion are prepared using the crystalline hemi-hydrate hemi-sulfate salt Form H, the crystalline DL-tartrate salt Form C-1, the crystalline DL-tartrate salt Form F, the crystalline DL-tartrate salt Form G, the crystalline hydrochloride salt Form D (i.e., Forms D, D-1, and D-2), the crystalline hydrochloride salt Form J, the crystalline hydrochloride salt Form K, or the crystalline L-tartrate salt Form I.

Alternatively, as part of a forty-eighth embodiment, the tablets comprise at least one crystalline form of Compound 1 as described herein. In one aspect the tablets comprise crystalline hemi-hydrate hemi-sulfate salt Form A. In another aspect the tablets comprise crystalline phosphate salt Form B. In another aspect the tablets comprise crystalline DL-tartrate salt Form C. In still another aspect the tablets comprise crystalline hydrochloride salt Form D. In yet another aspect the tablets comprise crystalline monohydrate free base Form E. In still another aspect the tablets comprise amorphous Compound 1.

Alternatively, as part of a forty-eighth embodiment, the tablets comprise at least one crystalline form of Compound 1 as described herein. In one aspect the tablets comprise the crystalline hemi-hydrate hemi-sulfate salt Form H, the crystalline DL-tartrate salt Form C-1, the crystalline DL-tartrate salt Form F, the crystalline DL-tartrate salt Form G, the crystalline L-tartrate salt Form I, the crystalline hydrochloride salt Form D (i.e., Forms D, D-1, and D-2), the crystalline hydrochloride salt Form J, the crystalline hydrochloride salt Form K, or crystalline L-tartrate salt Form I. In still another aspect the tablets comprise amorphous Compound 1.

In another alternative, as part of a forty-eighth embodiment, the tablets comprise amorphous Compound 1. In one aspect, the tablets comprise an amorphous solid dispersion of Compound 1. In another aspect, the tablets comprise an amorphous solid dispersion of a salt of Compound 1.

In a forty-ninth embodiment, capsules comprising the spray dried dispersions, comprise about 55% by weight, about 50% by weight, about 45% by weight, about 40% by weight, about 35%, by weight, about 30% by weight, about 25% by weight, or about 20% by weight of Compound 1. In one aspect the capsules comprising a spray dried dispersion are prepared using at least one of the crystalline forms described herein. In another aspect the capsules comprising a spray dried dispersion are prepared using crystalline hemi-hydrate hemi-sulfate salt Form A. In another aspect the capsules comprising the spray dried dispersion are prepared using crystalline phosphate salt Form B. In another aspect the capsules comprising the spray dried dispersion are prepared using the crystalline hemi-hydrate hemi-sulfate salt Form A, the crystalline phosphate salt Form B, the crystalline DL-tartrate salt form C, the crystalline hydrochloride salt Form D, or the crystalline free base monohydrate Form E.

In another alternative, as part of the forty-ninth embodiment, the capsules comprising the spray dried dispersion are prepared using the crystalline hemi-hydrate hemi-sulfate salt Form H, the crystalline DL-tartrate salt Form C-1, the crystalline DL-tartrate salt Form F, the crystalline DL-tartrate salt Form G, the crystalline hydrochloride salt Form D (i.e., Forms D, D-1, and D-2), the crystalline hydrochloride salt Form J, the crystalline hydrochloride salt Form K, or the crystalline L-tartrate salt Form I.

Alternatively, as part of a forty-ninth embodiment, the capsules comprise at least one crystalline form of Compound 1 as described herein. In one aspect the capsules comprise crystalline hemi-hydrate hemi-sulfate salt Form A. In another aspect the capsules comprise crystalline phosphate salt Form B. In another aspect the capsules comprise crystalline DL-tartrate salt Form C. In still another aspect the capsules comprise crystalline hydrochloride salt Form D. In yet another aspect the capsules comprise crystalline monohydrate free base Form E. In still another aspect the capsules comprise amorphous Compound 1.

Alternatively, as part of a forty-ninth embodiment, the capsules comprise at least one crystalline form of Compound 1 as described herein. In one aspect the capsules comprise the crystalline hemi-hydrate hemi-sulfate salt Form H, the crystalline DL-tartrate salt form C-1, the crystalline DL-tartrate salt Form F, the crystalline DL-tartrate salt Form G, the crystalline hydrochloride salt Form D (i.e., Forms D, D-1, and D-2), the crystalline hydrochloride salt Form J, the crystalline hydrochloride salt Form K, or crystalline L-tartrate salt Form I. In still another aspect the capsules comprise amorphous Compound 1.

In another alternative, as part of a forty-eighth embodiment, the capsules comprise amorphous Compound 1. In one aspect, the capsules comprise an amorphous solid dispersion of Compound 1. In another aspect, the capsules comprise an amorphous solid dispersion of a salt of Compound 1.

In a fiftieth embodiment, granules comprising the spray dried dispersions, comprise about 55% by weight, about 50% by weight, about 45% by weight, about 40% by weight, about 35%, by weight, about 30% by weight, about 25% by weight, or about 20% by weight of Compound 1. In one aspect the granules comprising a spray dried dispersion are prepared using at least one of the crystalline forms described herein. In another aspect the granules comprising a spray dried dispersion are prepared using crystalline hemi-hydrate hemi-sulfate salt Form A. In another aspect the granules comprising the spray dried dispersion are prepared using crystalline phosphate salt Form B. In another aspect the granules comprising the spray dried dispersion are prepared using the crystalline hemi-hydrate hemi-sulfate salt Form A, the crystalline phosphate salt Form B, the crystalline DL-tartrate salt form C, the crystalline hydrochloride salt Form D, or the crystalline free base monohydrate Form E.

In another alternative, as part of the fiftieth embodiment, the granules comprising the spray dried dispersion are prepared using the crystalline hemi-hydrate hemi-sulfate salt Form H, the crystalline DL-tartrate salt Form C-1, the crystalline DL-tartrate salt Form F, the crystalline DL-tartrate salt Form G, the crystalline hydrochloride salt Form D (i.e., Forms D, D-1, and D-2), the crystalline hydrochloride salt Form J, the crystalline hydrochloride salt Form K, or the crystalline L-tartrate salt Form I.

Alternatively, as part of a fiftieth embodiment, the granules comprise at least one crystalline form of Compound 1 as described herein. In one aspect the granules comprise crystalline hemi-hydrate hemi-sulfate salt Form A. In another aspect the granules comprise crystalline phosphate salt Form B. In another aspect the granules comprise crystalline DL-tartrate salt Form C. In still another aspect the granules comprise crystalline hydrochloride salt Form D. In yet another aspect the granules comprise crystalline monohydrate free base Form E. In still another aspect the granules comprise amorphous Compound 1.

Alternatively, as part of a fiftieth embodiment, the granules comprise at least one crystalline form of Compound 1 as described herein. In one aspect the granules comprise the crystalline hemi-hydrate hemi-sulfate salt Form H, the crystalline DL-tartrate salt Form C-1, the crystalline DL-tartrate salt Form F, the crystalline DL-tartrate salt Form G, the crystalline hydrochloride salt Form D (i.e., Forms D, D-1, and D-2), the crystalline hydrochloride salt Form J, the crystalline hydrochloride salt Form K, or crystalline L-tartrate salt Form I. In still another aspect the granules comprise amorphous Compound 1.

In another alternative, as part of a forty-eighth embodiment, the granules comprise amorphous Compound 1. In one aspect, the granules comprise an amorphous solid dispersion of Compound 1. In another aspect, the granules comprise an amorphous solid dispersion of a salt of Compound 1.

Alternatively, as part of a fifty-first embodiment, is provided a solid oral dosage form comprising one or more crystalline forms of Compound 1 as described herein. In one aspect the solid oral dosage form comprises crystalline hemi-hydrate hemi-sulfate salt Form A. In another aspect the solid oral dosage form comprises crystalline phosphate salt Form B. In another aspect the solid oral dosage form comprises crystalline DL-tartrate salt Form C. In still another aspect the solid oral dosage form comprises crystalline hydrochloride salt Form D. In yet another aspect the solid oral dosage form comprises crystalline monohydrate free base Form E. In a further aspect the solid oral dosage form comprises amorphous Compound 1.

Alternatively, as part of a fifty-first embodiment, is provided a solid oral dosage form comprising one or more crystalline forms of Compound 1 as described herein. In another aspect the solid oral dosage form comprises crystalline hemi-hydrate hemi-sulfate salt Form H. In another aspect the solid oral dosage form comprises crystalline DL-tartrate salt Form C (i.e., C and C-1). In another aspect the solid oral dosage form comprises crystalline DL-tartrate salt Form F. In another aspect the solid oral dosage form comprises crystalline DL-tartrate salt Form G. In another aspect the solid oral dosage form comprises crystalline L-tartrate salt Form I. In still another aspect the solid oral dosage form comprises crystalline hydrochloride salt Form D (i.e., D, D-1, and D-2). In still another aspect the solid oral dosage form comprises crystalline hydrochloride salt Form J. In still another aspect the solid oral dosage form comprises crystalline hydrochloride salt Form K. In a further aspect the solid oral dosage form comprises amorphous Compound 1.

### Methods of Preparation

In fifty-second embodiment, provided herein is a method of forming a hemi-hydrate hemi-sulfate salt of Compound 1. In an alternative embodiment, provided herein is a method of forming a hemi-hydrate hemi-sulfate salt of a compound having the structure:

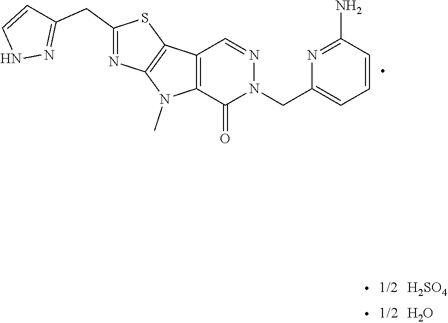

the method comprising reacting Compound 1:

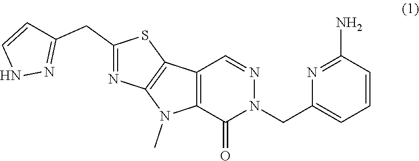 [SMILES: Cn1c2nc(Cc3cc[nH]n3)sc2c2cnn(Cc3cccc(N)n3)c(=O)c21]

with a solution comprising H₂SO₄.

In a fifty-third embodiment, the solution comprising H₂SO₄ used in the method described above in the fifty-second embodiment comprises an alcoholic solution comprising water, e.g. a solution comprising H₂SO₄, alcohol and water.

In a fifty-fourth embodiment, the H₂SO₄ solution used in the method described above in the fifty-third embodiment is part of an alcoholic solution comprising a ratio of alcohol to water of about 2:2, about 4:2, about 6:2, about 8:2, or about 10:2 v/v. Alternatively, as part of an fifty-fourth embodiment, the H₂SO₄ solution used in the method described above in the fifty-third embodiment is part of an alcoholic solution comprising a ratio of alcohol to water of about 8:2 v/v.

In a fifty-fifth embodiment, the alcohol described in the fifty-third or fifty-fourth embodiment is selected from methanol, ethanol, n-propanol, isopropyl alcohol, n-butanol, or isobutanol, propylene glycol, ethylene glycol, t-amyl alcohol, diethylene glycol, n-pentanol, benzyl alcohol, or cyclohexanol or any combination thereof. Alternatively, as part of a fifty-fifth embodiment, the alcohol described in the fifty-third or fifty-fourth embodiment is n-propanol.

In a fifty-sixth embodiment, provided herein is a method of forming a phosphate salt of Compound 1. In an alternative embodiment provided herein is a method of forming a phosphate salt of a compound having the structure:

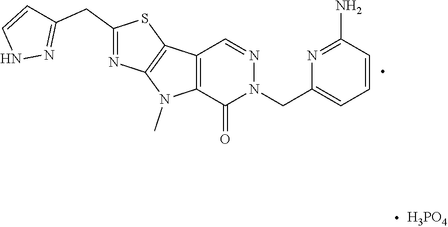 [SMILES: Cn1c2nc(Cc3cc[nH]n3)sc2c2cnn(Cc3cccc(N)n3)c(=O)c21 ; "H3PO4"]

the method comprising reacting Compound 1:

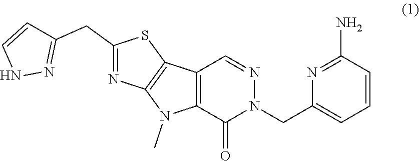 [SMILES: Cn1c2nc(Cc3cc[nH]n3)sc2c2cnn(Cc3cccc(N)n3)c(=O)c21]

with a solution comprising H₃PO₄ and at least one polar aprotic solvent.

In a fifty-seventh embodiment, the polar aprotic solvent used in the method described above in the fifty-sixth embodiment is selected from acetone, acetonitrile, dichloromethane, dimethylformamide, dimethyl sulfoxide, pyridine, or tetrahydrofuran, or any combination thereof. Alternatively, as part of the fifty-seventh embodiment, the polar aprotic solvent described in the fifty-sixth embodiment is dimethylsulfoxide.

In a fifty-eighth embodiment, the method described in the fifty-sixth embodiment further comprises a second step of adding an antisolvent. Alternatively, as part of the fifty-eighth embodiment, the antisolvent used in the fifty-sixth embodiment is an alcoholic solution. In another alternate, as part of the fifty-eighth embodiment, the alcoholic solution used in the fifty-sixth embodiment further comprises water.

In a fifty-ninth embodiment, the ratio of alcohol to water as described in the fifty-eighth embodiment is about 2:2, about 4:2, about 6:2, about 8:2, or about 10:2 v/v. Alternatively, as part of the fifty-ninth embodiment, the ratio of alcohol to water as described in the fifty-eighth embodiment is about 8:2 v/v.

In a sixtieth embodiment, the alcohol as described in the fifty-eighth and fifty-ninth embodiments is selected from methanol, ethanol, n-propanol, isopropyl alcohol, n-butanol, or isobutanol, propylene glycol, ethylene glycol, t-amyl alcohol, diethylene glycol, n-pentanol, benzyl alcohol, or cyclohexanol or any combination thereof. Alternatively, as part of the sixtieth embodiment, the alcohol as described in the fifty-eighth and fifty-ninth embodiments is ethanol.

In a sixty-first embodiment, provided herein is a method of forming a DL-tartrate salt of Compound 1. In an alternative embodiment provided herein is a method of forming a DL-tartrate salt of a compound having the structure:

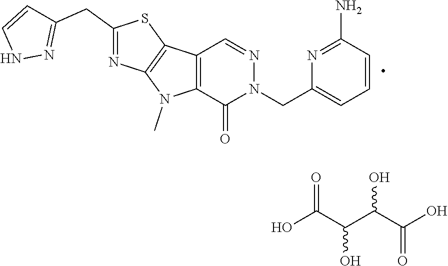 [SMILES: Cn1c2nc(Cc3cc[nH]n3)sc2c2cnn(Cc3cccc(N)n3)c(=O)c21 ; O=C(O)C(O)C(O)C(=O)O]

the method comprising reacting Compound 1:

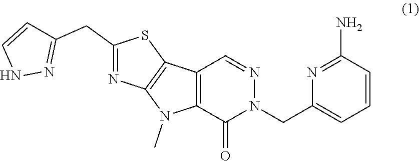 [SMILES: Cn1c2nc(Cc3cc[nH]n3)sc2c2cnn(Cc3cccc(N)n3)c(=O)c21]

with a solution comprising DL-tartaric acid and at least one polar aprotic solvent.In a sixty-second embodiment, the polar aprotic solvent used in the method described above in the sixty-first embodiment is selected from acetone, acetonitrile, dichloromethane, dimethylformamide, dimethyl sulfoxide, pyridine, or tetrahydrofuran, or any combination thereof. Alternatively, as part of the sixty-third embodiment, the polar aprotic solvent described in the sixty-first embodiment is tetrahydrofuran.

In a sixty-third embodiment, the solvent as described in the sixty-second embodiment, is a mixture further comprising water.

In a sixty-fourth embodiment, the ratio of solvent to water is about 99:1, about 95:5, about 90:10, about 80:20, or about 60:40 v/v. Alternatively, as part of the sixty-fourth, embodiment, the ratio of solvent to water as described in the is about 95:5 v/v.

In a sixty-fifth embodiment, provided herein is a method of forming a L-tartrate salt of Compound 1. In an alternative embodiment provided herein is a method of forming a L-tartrate salt of a compound having the structure:

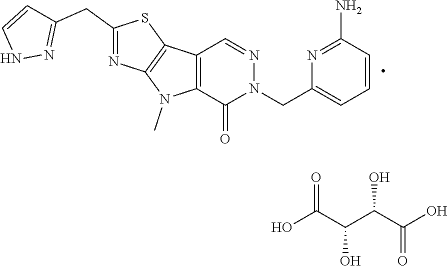 [SMILES: Cn1c2nc(Cc3cc[nH]n3)sc2c2cnn(Cc3cccc(N)n3)c(=O)c21 ; O=C(O)[C@@H](O)[C@H](O)C(=O)O]

the method comprising reacting Compound 1:

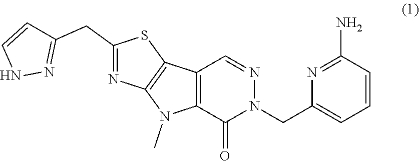 [SMILES: Cn1c2nc(Cc3cc[nH]n3)sc2c2cnn(Cc3cccc(N)n3)c(=O)c21]

with a solution comprising L-tartaric acid in a solution.

In a sixty-sixth embodiment, the solution comprising L-tartaric acid used in the method described above in the sixty-fifth embodiment comprises an alcoholic solution comprising water, e.g. a solution comprising L-tartaric acid, alcohol, and water.

In a sixty-seventh embodiment, the L-tartaric acid solution used in the method described above in the sixty-fifth embodiment is part of an alcoholic solution comprising a ratio of alcohol to water of about 99:1, about 95:5, about 90:10, about 80:20, or about 60:40 v/v. Alternatively, as part of an sixty-seventh embodiment, the L-tartaric acid solution used in the method described above in the sixty-fifth embodiment is part of an alcoholic solution comprising a ratio of alcohol to water of about 95:5 v/v.

In a sixty-eighth embodiment, the alcohol described in the sixty-sixth or sixty-seventh embodiment is selected from methanol, ethanol, n-propanol, isopropyl alcohol, n-butanol, or isobutanol, propylene glycol, ethylene glycol, t-amyl alcohol, diethylene glycol, n-pentanol, benzyl alcohol, or cyclohexanol or any combination thereof. Alternatively, as part of a sixty-eighth embodiment, the alcohol described in the sixty-sixth or sixty-seventh embodiment is ethanol.

In a sixty-ninth embodiment, provided herein is a method of forming a hydrochloride salt of Compound 1. In an alternative embodiment provided herein is a method of forming a hydrochloride salt of a compound having the structure:

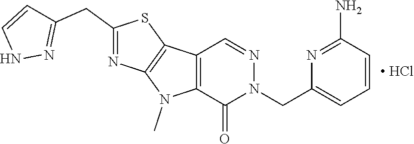 [SMILES: Cn1c2nc(Cc3cc[nH]n3)sc2c2cnn(Cc3cccc(N)n3)c(=O)c21 ; "· HCl"]

the method comprising reacting Compound 1:

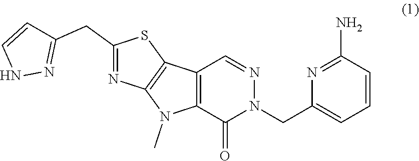 [SMILES: Cn1c2nc(Cc3cc[nH]n3)sc2c2cnn(Cc3cccc(N)n3)c(=O)c21]

with a solution comprising hydrochloric acid and at least one polar aprotic solvent.

In a seventieth embodiment, the polar aprotic solvent used in the method described above in the sixty-ninth embodiment is selected from acetone, acetonitrile, dichloromethane, dimethylformamide, dimethyl sulfoxide, pyridine, or tetrahydrofuran, or any combination thereof. Alternatively, as part of the seventieth embodiment, the polar aprotic solvent described in the sixty-ninth embodiment is tetrahydrofuran.

### Compositions and Administration

Provided herein are pharmaceutical compositions comprising one or more of the disclosed crystalline forms (e.g. crystalline Form A, B, C, or D) or an amorphous form of Compound 1 together with a pharmaceutically acceptable excipient. The amount of crystalline or amorphous form in a provided composition is an amount that is effective to measurably activate PKR in a subject.

Also provided herein are pharmaceutical compositions comprising one or more of the disclosed crystalline forms (e.g. crystalline Form C-1, D-1, D-2, E, F, G, H, I, J, or K) of Compound 1 together with a pharmaceutically acceptable excipient. The amount of crystalline form in a provided composition is an amount that is effective to measurably activate PKR in a subject.

As used herein, the term “composition” is used interchangeably with the term “pharmaceutical composition” and are comprised of the crystalline forms (e.g. crystalline Form A, B, C, or D) or amorphous forms of Compound 1, including salts, solvates, hydrates, anhydrous, and non-solvated forms, and zero, one, or more excipients as described herein.

As used herein, the term “composition” is used interchangeably with the term “pharmaceutical composition” and are comprised of the crystalline forms (e.g. crystalline Form C-1, D-1, D-2, E, F, G, H, I, J, or K) of Compound 1, including salts, solvates, hydrates, anhydrous, and non-solvated forms, and zero, one, or more excipients as described herein.

The pharmaceutical compositions described herein can be prepared by any method known in the art of pharmacology. In general, such preparatory methods include the steps of bringing one or more of the disclosed crystalline forms (e.g. crystalline Form A, B, C, or D) or an amorphous form of Compound 1 into association with at least one excipient and/or one or more other accessory ingredients, and then, if necessary and/or desirable, filling, shaping (by any number of methods) the mixture into a dosage form and/or packaging the product into a desired single- or multi-dose unit.

The pharmaceutical compositions described herein can be prepared by any method known in the art of pharmacology. In general, such preparatory methods include the steps of bringing one or more of the disclosed crystalline forms (e.g. crystalline Form C-1, D-1, D-2, E, F, G, H, I, J, or K) of Compound 1 into association with at least one excipient and/or one or more other accessory ingredients, and then, if necessary and/or desirable, filling, shaping (by any number of methods) the mixture into a dosage form and/or packaging the product into a desired single- or multi-dose unit.

In some embodiments, the pharmaceutical composition may be a pharmaceutical composition suitable for oral consumption or oral administration. Pharmaceutical compositions containing a compound of the disclosure suitable for oral administration can be presented as discrete dosage forms, such as capsules, cachets, or tablets, or as liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the crystalline form (e.g. crystalline Form A, B, C, or D) of the disclosure into association with an excipient, such as a carrier, which constitutes one or more necessary ingredients. Such dosage forms can also be prepared by any of the methods of pharmacy, but all methods include the step of bringing the crystalline form (e.g. crystalline Form A, B, C, or D) of the disclosure into association with an excipient, such as a carrier, which constitutes one or more necessary ingredients.

In some embodiments, the pharmaceutical composition may be a pharmaceutical composition suitable for oral consumption or oral administration. Pharmaceutical compositions containing a compound of the disclosure suitable for oral administration can be presented as discrete dosage forms, such as capsules, cachets, or tablets, or as liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the crystalline form (e.g. crystalline Form C-1, D-1, D-2, E, F, G, H, I, J, or K) of the disclosure into association with an excipient, such as a carrier, which constitutes one or more necessary ingredients. Such dosage forms can also be prepared by any of the methods of pharmacy, but all methods include the step of bringing the crystalline form (e.g. crystalline Form C-1, D-1, D-2, E, F, G, H, I, J, or K) of the disclosure into association with an excipient, such as a carrier, which constitutes one or more necessary ingredients.

In general, the compositions are prepared by uniformly and intimately admixing the crystalline form of the disclosure with liquid carriers or finely divided solid carriers or both, and then, if necessary, shaping the product into the desired presentation. For example, a tablet can be prepared by compression or molding, optionally with one or more accessory ingredients (excipients). Compressed tablets can be prepared by compressing in a suitable machine the active ingredient in a free-flowing form such as powder or granules, optionally mixed with one or more excipients such as, but not limited to, a binder, a lubricant, an inert diluent, and/or a surface active or dispersing agent. Molded tablets can be made by molding in a suitable machine a mixture of the powdered compound moistened with an inert liquid diluent.

The crystalline forms of the disclosure can be combined in an intimate admixture with one or more pharmaceutical excipients according to conventional pharmaceutical compounding techniques. The excipients can take a wide variety of forms depending on the form of preparation desired for administration. In preparing the compositions for an oral dosage form, any of the usual pharmaceutical media can be employed as excipients, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable oral dosage forms include powders, capsules, and tablets. If desired, tablets can be coated by standard aqueous or nonaqueous techniques. Carriers such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, silicified microcrystalline cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, silicified microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Examples of suitable fillers for use in the pharmaceutical compositions and dosage forms disclosed herein include, but are not limited to, talc, calcium carbonate (e.g., granules or powder), silicified microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 2 weight percent of the pharmaceutical composition.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Film coatings may be used, for example, Opadry® II Blue film coat [polyvinyl alcohol, titatnium dioxide, macrogol/polyethylene glycol, talc, FD&C blue #2/indigo carmine aluminum lake/E132].

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical, and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-1000 succinate, PEG-24 cholesterol, polyglyceryl-10-oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the disclosure and to minimize precipitation of the compound of the disclosure. This can be important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol (PEG), polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; polyethylene glycol 660 12-hydroxystearate, amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, glycofurol, transcutol, propylene glycol, and dimethyl isosorbide. Particularly preferred solubilizers include sorbitol, glycerol, triacetin, ethyl alcohol, PEG having an average molecular weight of about 100 to about 8000 g/mole, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of less than about 10%, less than about 25%, less than about 50%, about 100%, or up to less than about 200% by weight, based on the combined weight of the drug, and other excipients. If desired, very small amounts of solubilizer may also be used, such as less than about 5%, less than about 2%, less than about 1% or even less. Typically, the solubilizer may be present in an amount of less than about 1% to about 100%, more typically less than about 5% to less than about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

Compositions described herein may be administered orally, parenterally, by inhalation spray, topically, rectally, nasally, buccally, transmucosally, or in an ophthalmic preparation. The term “parenteral” as used herein includes subcutaneous, intravenous, intramuscular, intra-articular, intra-synovial, intrasternal, intrathecal, intrahepatic, intralesional and intracranial injection or infusion techniques. In one aspect, the pharmaceutical compositions provided herewith are orally administered in an orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

In one embodiment, the excipient is selected from one or more of mannitol, sorbitol, inositol, silicified microcrystalline cellulose, Croscarmellose Sodium, cross-linked poly(vinyl-pyrrolidone) (crospovidone), sodium carboxymethyl starch (sodium starch glycolate), pregelatinized starch (starch 1500), microcrystalline starch, Sodium Stearyl Fumarate, hydrogenated vegetable oil (e.g., peanut oil, cottonseed oil, sunflower oil, sesame oil, olive oil, corn oil, and soybean oil), zinc stearate, ethyl oleate, ethyl laureate, agar, polyvinyl alcohol, titatnium dioxide, macrogol/polyethylene glycol, sorbitan fatty acid esters, talc, FD&C blue #2/indigo carmine aluminum lake/E132.

In one embodiment, the excipient is selected from one or more of silicified microcrystalline cellulose, Croscarmellose Sodium, Sodium Stearyl Fumarate, polyvinyl alcohol, titatnium dioxide, macrogol/polyethylene glycol, talc, FD&C blue #2/indigo carmine aluminum lake/E132.

In one embodiment, the excipient is selected from one or more of silicified microcrystalline cellulose, croscarmellose Sodium, Sodium Stearyl Fumarate, and Opadry® II Blue film coat.

In one embodiment, the excipient is selected from one or more of mannitol, pregelatinized starch (starch 1500), hydrogenated vegetable oil, and Opadry® II Blue film coat.

In one embodiment, the excipient is selected from one or more of sorbitol, cross-sodium carboxymethyl starch (sodium starch glycolate), zinc stearate, and Opadry® II Blue film coat.

In one embodiment, the excipient is selected from one or more of silicified microcrystalline cellulose, Croscarmellose Sodium, Sodium Stearyl Fumarate, polyvinyl alcohol, titatnium dioxide, macrogol/polyethylene glycol, talc, FD&C blue #2/indigo carmine aluminum lake/E132, olyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol.

The amount of a crystalline or amorphous form of Compound 1 that may be combined with one or more excipients to produce a composition in a single dosage form will vary depending upon the subject to be treated and the particular mode of administration. For example, a specific dosage and treatment regimen for any particular subject will depend upon a variety of factors, including age, body weight, general health, sex, diet, time of administration, rate of excretion, drug combination, the judgment of the treating physician, and the severity of the particular disease being treated. The amount of a crystalline or amorphous form of Compound 1 in the composition will also depend upon the particular crystalline form (e.g., Form A, B, C, D, or E) in the composition. Alternatively, the amount of a crystalline or amorphous form of Compound 1 in the composition will also depend upon the particular crystalline form (e.g. crystalline Form C-1, D-1, D-2, F, G, H, I, J, or K) in the composition. As used herein, the dose refers to the amount of Compound 1 in a particular crystalline form. The amount of the particular crystalline form will be calculated based on the equivalence to the non-hydrated free-base (non-salt) form of Compound 1.

In one aspect, a disclosed crystalline form (e.g., crystalline Form A, B, C, D, or E) or amorphous form of Compound 1 is formulated for administration at a dose that is equivalent to about 0.5 mg to about 30 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 0.5 mg to about 30 mg of the non-hydrated free base (non-salt) form of Compound 1. In some embodiments, a disclosed crystalline form (e.g., crystalline Form A, B, C, D, or E) or amorphous form of Compound 1 is formulated for administration at a dose that is equivalent to about 0.5 mg to about 10 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 0.5 mg to about 10 mg of the non-hydrated free base (non-salt) form of Compound 1. In other embodiments a disclosed crystalline form (e.g., crystalline Form A, B, C, D, or E) or amorphous form of Compound 1 is formulated for administration at a dose that is equivalent to about 0.5 mg to about 7 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 0.5 mg to about 7 mg of the non-hydrated free base (non-salt) form of Compound 1. In other embodiments, a disclosed crystalline form (e.g., crystalline Form A, B, C, D, or E) or amorphous form of Compound 1 is formulated for administration at a dose that is equivalent to about 0.5 mg to about 5 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 0.5 mg to about 5 mg of the non-hydrated free base (non-salt) form of Compound 1. In other embodiments, a disclosed crystalline form (e.g., crystalline Form A, B, C, D, or E) or amorphous form of Compound 1 is formulated for administration at a dose that is equivalent to about 1 mg to about 5 mg of the non-hydrated free base (non-salt) form of Compound 1. In certain embodiments, the dose is oral dose. In a particular embodiment, a crystalline form (e.g., crystalline Form C-1, D-1, D-2, F, G, H, I, J, or K) is formulated for administration at a dose that is equivalent to about 1 mg to about 5 mg of the non-hydrated free base (non-salt) form of Compound 1. In certain embodiments, a disclosed crystalline form (e.g., crystalline Form A, B, C, D, or E) or amorphous form of Compound 1 is formulated for administration at a dose that is equivalent to about 1 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 1 mg of the non-hydrated free base (non-salt) form of Compound 1. In certain embodiments, a disclosed crystalline form (e.g., crystalline Form A, B, C, D, or E) or amorphous form of Compound 1 is formulated for administration at a dose that is equivalent to about 2 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 2 mg of the non-hydrated free base (non-salt) form of Compound 1. In certain embodiments, a disclosed crystalline form (e.g., crystalline Form A, B, C, D, or E) or amorphous form of Compound 1 is formulated for administration at a dose that is equivalent to about 5 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 5 mg of the non-hydrated free base (non-salt) form of Compound 1. In certain embodiments, a disclosed crystalline form (e.g., crystalline Form A, B, C, D, or E) or amorphous form of Compound 1 is formulated for administration at a dose that is equivalent to about 10 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 10 mg of the non-hydrated free base (non-salt) form of Compound 1. In certain embodiments a disclosed crystalline form (e.g., crystalline Form A, B, C, D, or E) or amorphous form of Compound 1 is formulated for administration at a dose that is equivalent to about 15 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 15 mg of the non-hydrated free base (non-salt) form of Compound 1. In other certain embodiments a disclosed crystalline form (e.g., crystalline Form A, B, C, D, or E) or amorphous form of Compound 1 is formulated for administration at a dose that is equivalent to about 20 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 20 mg of the non-hydrated free base (non-salt) form of Compound 1. In further embodiments a disclosed crystalline form (e.g., crystalline Form A, B, C, D, or E) or amorphous form of Compound 1 is formulated for administration at a dose that is equivalent to about 25 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 25 mg of the non-hydrated free base (non-salt) form of Compound 1. In still further embodiments a disclosed crystalline form (e.g., crystalline Form A, B, C, D, or E) or amorphous form of Compound 1 is formulated for administration at a dose that is equivalent to about 30 mg of the non-hydrated free base (non-salt) form of Compound 1. In a particular embodiment, a crystalline form (e.g., crystalline Form C-1, D-1, D-2, F, G, H, I, J, or K) is formulated for administration at a dose that is equivalent to about 30 mg of the non-hydrated free base (non-salt) form of Compound 1.

In some embodiments, crystalline Form A, B, C, D, or E is present in the tablet composition in an amount equivalent to about 1 to about 20 mg of Compound 1. In a particular embodiment, crystalline Form C-1, D-1, D-2, F, G, H, I, J, or K is present in the tablet composition in an amount equivalent to about 1 to about 20 mg of Compound 1. In some embodiments, crystalline Form A, B, C, D, or E is present in the tablet composition in an amount equivalent to about 0.5 to about 10 mg of Compound 1. In a particular embodiment, crystalline Form C-1, D-1, D-2, F, G, H, I, J, or K is present in the tablet composition in an amount equivalent to about 0.5 to about 10 mg of Compound 1. In some embodiments, crystalline Form A, B, C, D, or E is present in the tablet composition in an amount equivalent to about 0.5 to about 6 mg of Compound 1. In a particular embodiment, crystalline Form C-1, D-1, D-2, F, G, H, I, J, or K is present in the tablet composition in an amount equivalent to about 0.5 to about 6 mg of Compound 1. In some embodiments, crystalline Form A, B, C, D, or E is present in the tablet composition in an amount equivalent to about 0.5 to about 10 mg of Compound 1. In a particular embodiment, crystalline Form C-1, D-1, D-2, F, G, H, I, J, or K is present in the tablet composition in an amount equivalent to about 0.5 to about 10 mg of Compound 1. In some embodiments, crystalline Form A, B, C, D, or E is present in the tablet composition in an amount equivalent to about 0.5 mg of Compound 1. In a particular embodiment, crystalline Form C-1, D-1, D-2, F, G, H, I, J, or K is present in the tablet composition in an amount equivalent to about 0.5 mg of Compound 1. In some embodiments, crystalline Form A, B, C, D, or E is present in the tablet composition in an amount equivalent to about 1 to about 5 mg of Compound 1. In a particular embodiment, crystalline Form C-1, D-1, D-2, F, G, H, I, J, or K is present in the tablet composition in an amount equivalent to about 1 to about 5 mg of Compound 1. In some embodiments, crystalline Form A, B, C, D, or E is present in the tablet composition in an amount equivalent to about 1 mg of Compound 1. In a particular embodiment, crystalline Form C-1, D-1, D-2, F, G, H, I, J, or K I is present in the tablet composition in an amount equivalent to about 1 mg of Compound 1. In some embodiments, crystalline Form A, B, C, D, or E is present in the tablet composition in an amount equivalent to about 2 mg of Compound 1. In a particular embodiment, crystalline Form C-1, D-1, D-2, F, G, H, I, J, or K is present in the tablet composition in an amount equivalent to about 2 mg of Compound 1. In some embodiments, crystalline Form A, B, C, D, or E is present in the tablet composition in an amount equivalent to about 5 mg of Compound 1. In a particular embodiment, crystalline Form C-1, D-1, D-2, F, G, H, I, J, or K is present in the tablet composition in an amount equivalent to about 1 mg of Compound 1. In still other embodiments, crystalline Form A, B, C, D, or E is present in the tablet composition in an amount equivalent to about 7 mg of Compound 1. In a particular embodiment, crystalline Form C-1, D-1, D-2, F, G, H, I, J, or K is present in the tablet composition in an amount equivalent to about 7 mg of Compound 1. In still further embodiments, crystalline Form A, B, C, D, or E is present in the tablet composition in an amount equivalent to about 10 mg of Compound 1. In a particular embodiment, crystalline Form C-1, D-1, D-2, F, G, H, I, J, or K is present in the tablet composition in an amount equivalent to about 10 mg of Compound 1.

As used herein, the amount of crystalline Form A, B, C, D, or E (or amorphous form of Compound 1) is based on the equivalence to the non-hydrated free-base form of Compound 1. Alternatively, as used herein, the amount of crystalline Form C-1, D-1, D-2, F, G, H, I, J, or K (or amorphous form of Compound 1) is based on the equivalence to the non-hydrated free-base form of Compound 1. For example, “crystalline Form A is present in the composition in an amount equivalent to about 1.0 mg of Compound 1” means that about 1.18 mg of crystalline Form A is present in the composition and is equivalent to about 1.0 mg of the non-hydrated free base (non-salt) form of Compound 1.

In one aspect, the tablet composition comprises about 2% w/w (±1%) of a crystalline or amorphous form of Compound 1 and about 98% w/w (±2%) comprising one or more excipients. In the foregoing tablet composition any of crystalline forms A, B, C, D, and E as described herein may be used. Alternatively, in the foregoing tablet composition any of crystalline forms C-1, D-1, D-2, F, G, H, I, J, or K as described herein may be used.

In one aspect, the tablet composition comprises about 5% w/w (±1%) of a crystalline (any of crystalline Form A, B, C, D, or E, as described herein) or amorphous form of Compound 1 and about 95% w/w (±2%) comprising one or more excipients.

In one aspect, the tablet composition comprises about 5% w/w (±1%) of a crystalline (any of crystalline Form C-1, D-1, D-2, F, G, H, I, J, or K, as described herein) or amorphous form of Compound 1 and about 95% w/w (±2%) comprising one or more excipients.

In one aspect, the tablet composition comprises about 7% w/w (±1%) of a crystalline (any of crystalline Form A, B, C, D, or E, as described herein) or amorphous form of Compound 1 and about 93% w/w (±2%) comprising one or more excipients.

In one aspect, the tablet composition comprises about 7% w/w (±1%) of a crystalline (any of crystalline Form C-1, D-1, D-2, F, G, H, I, J, or K, as described herein) or amorphous form of Compound 1 and about 93% w/w (±2%) comprising one or more excipients.

In one aspect, the tablet composition comprises about 10% w/w (±1%) of a crystalline (any of crystalline Form A, B, C, D, or E as described herein) or amorphous form of Compound 1 and about 90% w/w (±2%) comprising one or more excipients.

In one aspect, the tablet composition comprises about 10% w/w (±1%) of a crystalline (any of crystalline Form C-1, D-1, D-2, F, G, H, I, J, or K as described herein) or amorphous form of Compound 1 and about 90% w/w (±2%) comprising one or more excipients.

In one aspect, the tablet composition comprises about 12% w/w (±1%) of a crystalline (any of crystalline Form A, B, C, D, or E as described herein) or amorphous form of Compound 1 and about 88% w/w (±2%) comprising one or more excipients.

In one aspect, the tablet composition comprises about 12% w/w (±1%) of a crystalline (any of crystalline Form C-1, D-1, D-2, F, G, H, I, J, or K as described herein) or amorphous form of Compound 1 and about 88% w/w (±2%) comprising one or more excipients.

In one aspect, the tablet composition comprises about 15% w/w (±1%) of a crystalline (any of crystalline Form A, B, C, D, or E as described herein) or amorphous form of Compound 1 and about 85% w/w (±2%) comprising one or more excipients.

In one aspect, the tablet composition comprises about 15% w/w (±1%) of a crystalline (any of crystalline Form C-1, D-1, D-2, F, G, H, I, J, or K as described herein) or amorphous form of Compound 1 and about 85% w/w (±2%) comprising one or more excipients.

In one aspect, the tablet composition comprises about 18% w/w (±1%) of a crystalline (any of crystalline Form A, B, C, D, or E as described herein) or amorphous form of Compound 1 and about 82% w/w (±2%) comprising one or more excipients.

In one aspect, the tablet composition comprises about 18% w/w (±1%) of a crystalline (any of crystalline Form C-1, D-1, D-2, F, G, H, I, J, or K as described herein) or amorphous form of Compound 1 and about 82% w/w (±2%) comprising one or more excipients.

In one aspect, the tablet composition comprises about 20% w/w (±1%) of a crystalline (any of crystalline Form A, B, C, D, or E as described herein) or amorphous form of Compound 1 and about 80% w/w (±2%) comprising one or more excipients.

In one aspect, the tablet composition comprises about 20% w/w (±1%) of a crystalline (any of crystalline Form C-1, D-1, D-2, F, G, H, I, J, or K as described herein) or amorphous form of Compound 1 and about 80% w/w (±2%) comprising one or more excipients.

In one aspect, the tablet composition comprises about 25% w/w (±1%) of a crystalline (any of crystalline Form A, B, C, D, or E as described herein) or amorphous form of Compound 1 and about 75% w/w (±2%) comprising one or more excipients.

In one aspect, the tablet composition comprises about 25% w/w (±1%) of a crystalline (any of crystalline Form C-1, D-1, D-2, F, G, H, I, J, or K as described herein) or amorphous form of Compound 1 and about 75% w/w (±2%) comprising one or more excipients.

In certain embodiments, provided is a pharmaceutical composition comprising any of the crystalline forms Form A, B, C, D, or E or an amorphous form of Compound 1 as described herein and a pharmaceutically acceptable excipient that has been processed to generate particles of a consistent size (“milled powder”). In other embodiments, provided is a pharmaceutical composition comprising crystalline Form C-1, D-1, D-2, F, G, H, I, J, or K, as described herein and a pharmaceutically acceptable excipient that has been processed to generate particles of a consistent size (“milled powder”). In certain embodiments, processing the milled powder comprises milling for an amount of time suitable to bring about a desired particle size. In some embodiments, the particle size of the milled powder is less than about 200 μm. In some embodiments, the particle size of the milled powder is less than about 100 μm. In some embodiments, the particle size of the milled powder is less than about 75 μm. In some embodiments, the particle size of the milled powder is less than about 50 μm. In some embodiments, the particle size of the milled powder is less than about 45 μm. In some embodiments, the particle size of the milled powder is less than about 40 μm. In some embodiments, the particle size of the milled powder is less than about 35 μm. In some embodiments, the particle size of the milled powder is less than about 30 μm. In some embodiments, the particle size of the milled powder is less than about 25 μm. In some embodiments, the particle size of the milled powder is less than about 20 μm. In some embodiments, the particle size of the milled powder is less than about 15 μm. In some embodiments, the particle size of the milled powder is less than about 10 μm. In some embodiments, the particle size of the milled powder is less than about 5 μm. In some embodiments, the particle size of the milled powder is less than about 1 μm. In some embodiments, the particle size of the milled powder is less than about 500 nm. In some embodiments, the particle size of the milled powder ranges from about 500 nm to about 200 μm. In some embodiments, the particle size of the milled powder ranges from about 500 nm to about 100 μm. In some embodiments, the particle size of the milled powder ranges from about 500 nm to about 75 μm. In some embodiments, the particle size of the milled powder ranges from about 500 nm to about 50 μm. In some embodiments, the particle size of the milled powder ranges from about 500 nm to about 45 μm. In some embodiments, the particle size of the milled powder ranges from about 500 nm to about 40 μm. In some embodiments, the particle size of the milled powder ranges from about 500 nm to about 35 μm. In some embodiments, the particle size of the milled powder ranges from about 1 μm to about 45 μm. In some embodiments, the particle size of the milled powder ranges from about 1 μm to about 40 μm. In some embodiments, the particle size of the milled powder ranges from about 1 μm to about 35 μm. In some embodiments, the particle size of the milled powder is about 45 μm. In some embodiments, the particle size of the milled powder is about 40 μm. In some embodiments, the particle size of the milled powder is about 35 μm. In some embodiments, the particle size of the milled powder is about 30 μm. In some embodiments, the particle size of the milled powder is about 25 μm. In some embodiments, the particle size of the milled powder is about 5 μm. In some embodiments, the particle size of the milled powder is about 4 μm. In some embodiments, the particle size of the milled powder is about 3 μm. In some embodiments, the particle size of the milled powder is about 2 μm. In some embodiments, the particle size of the milled powder is about 1 μm. The term “about,” as used herein with respect to particle size, means +/−5 μm.

In some embodiments, at least 90% of a representative sample of the milled powder has a particle size of less than about 100, about 80, about 70, about 60, about 50, about 40, about 30, about 20, or about 10 μm. In some embodiments, at least about 90% of a representative sample of the milled powder has a particle size of less than about 60 μm.

In some embodiments, the pharmaceutical composition comprises amorphous Compound 1. In some embodiments, the pharmaceutical composition comprises amorphous Compound 1 and at least one excipient. In some embodiments, the pharmaceutical composition comprises a solid dispersion. In still other embodiments, the pharmaceutical composition comprises amorphous Compound 1 in a spray dried dispersion.

In some embodiments, the solid dispersion or pharmaceutical composition containing the solid dispersion comprises Compound 1 or a salt thereof and one or more polymer(s). In other embodiments, the solid dispersion is a spray dried dispersion. In some embodiments, the solid dispersion comprises Compound 1 or a salt thereof, one or more polymer(s), and one or more surfactant(s). In some embodiments, the solid dispersion or pharmaceutical composition containing the solid dispersion comprises Compound 1 or a salt thereof and at least one polymer. In some embodiments, the solid dispersion or pharmaceutical composition containing the solid dispersion comprises Compound 1 or a salt thereof, at least one polymer, and at least one surfactant.

In certain embodiments, the non-salt (free form) of Compound 1 is used in the solid dispersion or pharmaceutical composition containing the solid dispersion. In other embodiments, a pharmaceutically acceptable salt of Compound 1 is used in the solid dispersion or pharmaceutical composition containing the solid dispersion. In certain embodiments, the amorphous form of Compound 1 is used in the solid dispersion or pharmaceutical composition containing the solid dispersion. In other embodiments, the solid dispersion or pharmaceutical composition containing the solid dispersion comprises the monohydrate free form of Compound 1. In other embodiments, the solid dispersion or pharmaceutical composition containing the solid dispersion comprises non-hydrated free form of Compound 1. In further embodiments, the pharmaceutically acceptable salt of Compound 1 used in the solid dispersion or pharmaceutical composition containing the solid dispersion is the phosphate salt of Compound 1. In yet other embodiments, the pharmaceutically acceptable salt of Compound 1 used in the solid dispersion or pharmaceutical composition containing the solid dispersion is the hemi-hydrate hemi-sulfate salt of Compound 1. In still further embodiments, the pharmaceutically acceptable salt of Compound 1 used in the solid dispersion or pharmaceutical composition containing the solid dispersion is the DL-tartrate salt of Compound 1. In even further embodiments, the pharmaceutically acceptable salt of Compound 1 used in the solid dispersion or pharmaceutical composition containing the solid dispersion is the hydrochloride salt of Compound 1. In further embodiments, the pharmaceutically acceptable salt of Compound 1 used in the solid dispersion or pharmaceutical composition containing the solid dispersion is the L-tartrate salt of Compound 1.

In some embodiments, the solid dispersion comprises at least one polymer that is a water-soluble polymer. In other embodiments, the solid dispersion comprises at least one polymer that is a cellulosic polymer. In further embodiments, the solid dispersion comprises at least one polymer that is a cellulose ether, cellulose ester, cellulose ω-carboxyester, cellulose phthalate, cellulose succinate, or mixtures thereof. In yet other embodiments, the solid dispersion comprises at least one polymer that is methylcellulose (MC); ethylcellulose (EC); hydroxyethylcellulose (HEC); hydroxypropyl methyl cellulose (HPMC) such as HPMC 606 or HPMC E5; hydroxypropyl cellulose (HPC); carboxymethyl ethyl cellulose (CMEC); hydroxypropyl methyl cellulose acetosuccinate (HPMCAS) such as HPMCAS-LG, HPMCAS-MG, HPMCAS-HG, HPMCAS/SLS, HPMCAS AS-MF, HPMCAS-HF; hydroxypropyl methyl cellulose phthalate (HPMCP); cellulose acetate phthalate (CAP); cellulose acetate groups having at least a half of cellulose acetate in hydrolyzed form; polyvinylpyrrolidone such as PVP K-12, PVPVA, PVP K30, PVP K 29/32, or PVPVA 64; polyoxyethylene-polyoxypropylene copolymers; polyvinylacetate (PVAc); poly(2-vinyl pyridine) (P2VP), TPGS, copovidone; cellulose acetate (CA); cellulose acetate butyrate (CAB); 5-carboxypentyl hydroxypropyl cellulose (CHC); polyacrylic acid (PAA); carboxymethylcellulose derivatives such as carboxymethyl cellulose (CMC) or carboxymethyl cellulose acetate butyrate (CMCAB); hydroxypropylmethylphthalate (HPMP); hydroxypropylmethylphthalate acetate succinate (HPMPAS); Poly(methylacrylic acid-co-ethyl acrylate) such as EUDRAGIT L100; Eudragit EPO; Eudragit E-100; cellulose acetate adipate (CAAdP); cellulose acetate suberate (CASub); methylcellulose adipate (MCAd); cellulose acetate butyrate sebacate (CAB Seb); cellulose acetate butyrate suberate (CAB Sub); cellulose acetate sebacate (CASeb); cellulose acetate phthalate (CAPhth); cellulose succinate (CS); cellulose acetate butyrate suberate (CABSu); HPCPen106-AA-H-Hydroxypropyl pent-4-enyl cellulose; HPC-SSL; HP-3-CD; or mixtures thereof. In further embodiments, the solid dispersion comprises one or more polymers selected from hydroxypropyl methyl cellulose (HPMC), hydroxypropyl methylcellulose acetate succinate (HPMCAS), hydroxypropyl methyl cellulose phthalate (HPMCP), hydroxypropyl cellulose (HPC), ethylcellulose, cellulose acetate phthalate, and polyvinylpyrrolidone (PVP), and mixtures thereof. In still further embodiments one or more polymers in the solid dispersion is a cellulose-based polymer such as HPMC, HPMCAS, HPC, and ethylcellulose. In other embodiments, at least one polymer in the solid dispersion is HPMCAS. In still other embodiments at least one polymer in the solid dispersion is HPMC. In yet further embodiments at least one polymer in the solid dispersion is PVP. In still further embodiments at least one polymer in the solid dispersion is ethylcellulose. In additional embodiments at least one polymer in the solid dispersion is copovidone.

In some embodiments, the polymer is (or the one or more polymers are) present in the solid dispersion in an amount of about 10% w/w to 90% w/w (e.g., about 20% w/w to about 80% w/w; about 30% w/w to about 70% w/w; about 40% w/w to about 60% w/w; or between about 15% w/w and about 35% w/w). In some embodiments, the polymer is (or the one or more polymers are) present in the solid dispersion in an amount of from about 10% w/w to about 80% w/w, for example from about 30% w/w to about 75% w/w, or from about 40% w/w to about 65% w/w, or from about 45% w/w to about 55% w/w, for example, about 46% w/w, about 47% w/w, about 48% w/w, about 49% w/w, about 50% w/w, about 51% w/w, about 52% w/w, about 53% w/w, or about 54% w/w. In some embodiments, the polymer is (or the one or more polymers are) present in the solid dispersion in an amount of about 48% w/w, about 48.5% w/w, about 49% w/w, about 49.5% w/w, about 50% w/w, about 50.5% w/w, about 51% w/w, about 51.5% w/w, about 52% w/w, or about 52.5% w/w.

In some embodiments, the solid state form of Compound 1, is present in the solid dispersion in an amount of from about 10% w/w and 90% w/w (equivalent to the amount of non-hydrated free form Compound 1) (e.g., about 20% w/w to about 80% w/w; about 30% w/w to about 70% w/w; about 40% w/w to about 60% w/w; or about 15% w/w to about 35% w/w). In some embodiments, the solid state form of Compound 1, is present in the solid dispersion in an amount of from about 10% w/w to about 90% w/w, for example from about 20% w/w to about 80% w/w, or from about 30% w/w to about 70% w/w. In other embodiments, the solid state form of Compound 1 is present in the solid dispersion in an amount of from about, for example, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 31% w/w, about 32% w/w, about 33% w/w, about 34% w/w, about 35% w/w, about 36% w/w, about 37% w/w, about 38% w/w, about 39% w/w, or about 40% w/w.

In some embodiments, the solid dispersion further comprises a surfactant. In some embodiments, the surfactant is selected from sodium lauryl sulfate (SLS), vitamin E or a derivative thereof (e.g., vitamin E TPGS), docusate sodium, sodium dodecyl sulfate, polysorbates (such as Tween 20 and Tween 80), poloxamers (such as Poloxamer 335 and Poloxamer 407), glyceryl monooleate, Span 65, Span 25, Capryol 90, pluronic copolymers (e.g., Pluronic F108, Pluronic P-123), and mixtures thereof. In some embodiments, the surfactant is SLS. In other embodiments, the surfactant is vitamin E or a derivative thereof (e.g., vitamin E TPGS).

In some embodiments, the surfactant is present in the solid dispersion in an amount of from about 0.1% w/w to about 10% w/w, for example from about 0.5% w/w to about 2% w/w, or from about 1% w/w to about 3% w/w, from about 1% w/w to about 4% w/w, or from about 1% w/w to about 5% w/w. In some embodiments, the surfactant is present in the solid dispersion in an amount of about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, or about 1% w/w. In some embodiments, the surfactant is present in the solid dispersion in an amount of about 0.5% w/w, about 1% w/w, about 1.5% w/w, about 2% w/w, about 2.5% w/w, about 3% w/w, about 3.5% w/w, about 4% w/w, about 4.5% w/w, or about 5% w/w.

In another aspect, the disclosure relates to processes for preparing a solid dispersion using any of the solid state forms disclosed herein. In some embodiments, the process comprises spray-drying a mixture prepared using a solid state form of Compound 1 as described herein, a polymer, and an appropriate solvent or solvent mixture. In other embodiments, the solid dispersion prepared according to the processes disclosed herein comprises Compound 1 (or a salt of Compound 1) in substantially amorphous form. In one embodiment, the process comprises combining a solid state form of Compound 1 (or a salt of Compound 1) as described herein with a polymer and a solvent to form a mixture that is an emulsion, solution, or suspension; and spray-drying the mixture to produce the solid dispersion. In certain aspects, the at least one polymer utilized is described above. In some embodiments, the solvent is methylene chloride, acetone, methanol, ethanol, chloroform, tetrahydrofuran (THF), or a mixture thereof. In other embodiments, the solvent is methylene chloride and methanol.

In some embodiments, a solid state form of Compound 1 as described herein may be used as the starting material in a process to prepare the solid dispersion. In some embodiments, the solid state form used as a starting material in the process to prepare the solid dispersion is one of the crystalline forms described herein. In some embodiments the solid dispersion is prepared by spray drying.

As used herein, the term “solid state form of Compound 1” refers to a crystalline salt form or a crystalline non salt form of Compound 1 (i.e.. crystalline Form A, B, C, D, or E) or an amorphous form of Compound 1.

As used herein, the term “solid state form of Compound 1” refers to a crystalline salt form or a crystalline non salt form of Compound 1 (i.e.. crystalline Form C-1, D-1, D-2, F, G, H, I, J, or K) or an amorphous form of Compound 1.

Spray drying involves atomization of a liquid solution containing, e.g., a solid and a solvent or solvent mixture, and removal of the solvent or solvent mixture. Atomization may be done, for example, through a two-fluid or pressure or electrosonic nozzle or on a rotating disk. Removal of the solvent or solvent mixture may require a subsequent drying step, such as tray drying, fluid bed drying (e.g., from about room temperature to about 100° C.), vacuum drying, microwave drying, rotary drum drying or biconical vacuum drying (e.g., from about room temperature to about 200° C.). Techniques and methods for spray-drying may be found in Perry's Chemical Engineering Handbook, 6th Ed., R. H. Perry, D. W. Green & J. O. Maloney, eds., McGraw-Hill Book Co. (1984); and Marshall “Atomization and Spray-Drying” 50, Chem. Eng. Prog. Monogr. Series 2 (1954).

### Methods of Treatment and Uses of Compounds and Compositions

In one embodiment, provided is a method for treating a disease, condition or disorder as described herein comprising administering crystalline Form A, B, C, D, or E or an amorphous form of Compound 1 as described herein or a pharmaceutical composition comprising crystalline Form A, B, C, D, or E or an amorphous form of Compound 1 as described herein and a pharmaceutically acceptable excipient.

In another embodiment, provided is a method for treating a disease, condition or disorder as described herein comprising administering crystalline Form C-1, D-1, D-2, F, G, H, I, J, or K as described herein or a pharmaceutical composition comprising crystalline Form C-1, D-1, D-2, F, G, H, I, J, or K as described herein and a pharmaceutically acceptable excipient.

In one embodiment provided is a method for increasing the lifetime of red blood cells (RBCs) in a subject in need thereof comprising administering an effective amount of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient.

In another embodiment provided is a method for increasing the lifetime of red blood cells (RBCs) in a subject in need thereof comprising administering an effective amount of (1) crystalline Form C-1, D-1, D-2, F, G, H, I, J, or K as described herein; or (2) a pharmaceutically acceptable composition comprising a crystalline Form C-1, D-1, D-2, F, G, H, I, J, or K as described herein and a pharmaceutically acceptable excipient.

In one embodiment provided is a method for reducing 2,3-diphosphoglycerate levels in the blood of a subject in need thereof comprising administering an effective amount of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient.

In one embodiment provided is a method for reducing 2,3-diphosphoglycerate levels in the blood of a subject in need thereof comprising administering an effective amount of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient.

In one embodiment provided is a method for increasing the level of hemoglobin (Hb) in a subject in need thereof comprising administering an effective amount of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient.

In one embodiment provided is a method for increasing the level of hemoglobin (Hb) in a subject in need thereof comprising administering an effective amount of (1) crystalline Form C-1, D-1, D-2, F, G, H, I, J, or K as described herein; or (2) a pharmaceutically acceptable composition comprising a crystalline Form C-1, D-1, D-2, F, G, H, I, J, or K as described herein and a pharmaceutically acceptable excipient.

In one embodiment provided is a method for treating sickle cell disease comprising administering to a subject in need thereof an effective amount of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient.

In one embodiment provided is a method for treating sickle cell disease comprising administering to a subject in need thereof an effective amount of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient.

In one embodiment provided is a method of treating acquired pyruvate kinase deficiency (PKD) in a subject comprising administering to the subject in need thereof an effective amount of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient.

In one embodiment provided is a method of treating acquired pyruvate kinase deficiency (PKD) in a subject comprising administering to the subject in need thereof an effective amount of (1) crystalline Form C-1, D-1, D-2, F, G, H, I, J, or Kas described herein; or (2) a pharmaceutically acceptable composition comprising crystalline Form C-1, D-1, D-2, F, G, H, I, J, or Kas described herein and a pharmaceutically acceptable excipient.

In one embodiment provided is a method of treating anemia in a subject comprising administering to the subject in need thereof an effective amount of (1) crystalline Form A, B, C, D, or E or an amorphous form of Compound 1 as described herein; or (2) a pharmaceutically acceptable composition comprising a crystalline Form A, B, C, D, or E or an amorphous form of Compound 1 as described herein and a pharmaceutically acceptable excipient. In certain embodiments, the anemia is hemolytic anemia. In certain embodiments, the hemolytic anemia is associated with MDS (e.g., very low risk MDS, low risk MDS, lower risk MDS, and/or intermediate risk MDS). In certain embodiments, the hemolytic anemia is acquired PKD associated with MDS (e.g., very low risk MDS, low risk MDS, lower risk MDS, and/or intermediate risk MDS). In certain embodiments is provided a method of treating anemia associated with MDS (e.g., very low risk MDS, low risk MDS, lower risk MDS, and/or intermediate risk MDS) in a subject comprising administering to the subject in need thereof an effective amount of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient.

In one embodiment provided is a method of treating anemia in a subject comprising administering to the subject in need thereof an effective amount of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient. In certain embodiments, the anemia is hemolytic anemia. In certain embodiments, the hemolytic anemia is associated with MDS (e.g., very low risk MDS, low risk MDS, lower risk MDS and/or intermediate risk MDS). In certain embodiments, the hemolytic anemia is acquired PKD associated with MDS (e.g., very low risk MDS, low risk MDS, lower risk MDS and/or intermediate risk MDS). In certain embodiments is provided a method of treating anemia associated with MDS (e.g., very low risk MDS, low risk MDS, lower risk MDS and/or intermediate risk MDS) in a subject comprising administering to the subject in need thereof an effective amount of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient.

As used herein, the term “anemia” refers to a deficiency of red blood cells (RBCs) and/or hemoglobin. As used herein, anemia includes all types of clinical anemia, for example (but not limited to): microcytic anemia, iron deficiency anemia, hemoglobinopathies, heme synthesis defect, globin synthesis defect, sideroblastic defect, normocytic anemia, anemia of chronic disease, aplastic anemia, hemolytic anemia, macrocytic anemia, megaloblastic anemia, pernicious anemia, dimorphic anemia, anemia of prematurity, Fanconi anemia, hereditary spherocytosis, sickle cell disease, warm autoimmune hemolytic anemia, cold agglutinin hemolytic anemia, osteopetrosis, thalassemia, and myelodysplastic syndrome. In some embodiments anemia refers to hemolytic anemia. In other embodiments anemia refers to hemolytic anemia associated with acquired PKD in patients with MDS (very low risk MDS, low risk MDS, lower risk MDS and/or intermediate risk MDS).

In certain embodiments, anemia can be diagnosed on a complete blood count. In certain embodiments, anemia can be diagnosed based on the measurement of one or more markers of hemolysis (e.g., RBC count, hemoglobin, reticulocytes, schistocytes, lactate Dehydrogenase (LDH), haptoglobin, bilirubin, and ferritin) and/or hemosiderinuria mean corpuscular volume (MCV) and/or red cell distribution width (RDW).

In one embodiment provided is a method for treating hemolytic anemia in a subject comprising administering to a subject in need thereof an effective amount of (1) crystalline Form A, B, C, D, or E or an amorphous form of Compound 1 as described herein; or (2) a pharmaceutically acceptable composition comprising a crystalline Form A, B, C, D, or E or an amorphous form of Compound 1 as described herein and a pharmaceutically acceptable excipient.

In one embodiment provided is a method for treating hemolytic anemia in a subject comprising administering to a subject in need thereof an effective amount of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for increasing the lifetime of red blood cells (RBCs) in a subject in need thereof.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for increasing the lifetime of red blood cells (RBCs) in a subject in need thereof.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for reducing 2,3-diphosphoglycerate levels in the blood of a subject in need thereof.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for reducing 2,3-diphosphoglycerate levels in the blood of a subject in need thereof.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for the preparation of a medicament for treating anemia in a subject in need thereof. In certain embodiments, the anemia is hemolytic anemia. In certain embodiments, the hemolytic anemia is a congenital and/or hereditary form of hemolytic anemia. In certain embodiments, the hemolytic anemia is acquired hemolytic anemia. In certain embodiments, the hemolytic anemia is anemia associated with MDS (very low risk MDS, low risk MDS, lower risk MDS and/or intermediate risk MDS). In other certain embodiments, the hemolytic anemia is associated with acquired PKD in a subject suffering from MDS (very low risk MDS, low risk MDS, lower risk MDS and/or intermediate risk MDS).

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for the preparation of a medicament for treating anemia in a subject in need thereof. In certain embodiments, the anemia is hemolytic anemia. In certain embodiments, the hemolytic anemia is a congenital and/or hereditary form of hemolytic anemia. In certain embodiments, the hemolytic anemia is acquired hemolytic anemia. In certain embodiments, the hemolytic anemia is anemia associated with MDS (very low MDS, low risk MDS, lower risk MDS and/or intermediate risk MDS). In other certain embodiments, the hemolytic anemia is associated with acquired PKD in a subject suffering from MDS (very low MDS, low risk MDS, lower risk MDS and/or intermediate risk MDS).

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein, and a pharmaceutically acceptable excipient for the preparation of a medicament for treating hemolytic anemia in a subject in need thereof.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein, and a pharmaceutically acceptable excipient for the preparation of a medicament for treating hemolytic anemia in a subject in need thereof.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for the preparation of a medicament for treating sickle cell disease in a subject in need thereof.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for the preparation of a medicament for treating sickle cell disease in a subject in need thereof.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for the preparation of a medicament for treating acquired pyruvate kinase deficiency (PKD) in a subject in need thereof.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for the preparation of a medicament for treating acquired pyruvate kinase deficiency (PKD) in a subject in need thereof.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for the preparation of a medicament for treating acquired hemolytic anemia.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for the preparation of a medicament for treating acquired hemolytic anemia.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for the preparation of a medicament for activating mutant or wild-type PKR in red blood cells in a subject in need thereof.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for the preparation of a medicament for activating mutant or wild-type PKR in red blood cells in a subject in need thereof.

In one embodiment provided is a use of (1) crystalline Form A, B, C, D, or E or an amorphous form of Compound 1 as described herein; or (2) a pharmaceutically acceptable composition comprising crystalline Form A, B, C, D, or E or an amorphous form of Compound 1 as described herein and a pharmaceutically acceptable excipient for the preparation of a medicament for treating anemia associated with MDS (very low risk MDS, low risk MDS, lower risk MDS and/or intermediate risk MDS) in a subject in need thereof.

In one embodiment provided is a use of (1) crystalline Form C-1, D-1, D-2, F, G, H, I, J, or K as described herein; or (2) a pharmaceutically acceptable composition comprising crystalline Form C-1, D-1, D-2, F, G, H, I, J, or K as described herein and a pharmaceutically acceptable excipient for the preparation of a medicament for treating anemia associated with MDS (very low risk MDS, low risk MDS, lower risk MDS and/or intermediate risk MDS) in a subject in need thereof.

### EXEMPLIFICATION

As depicted in the Examples below, the crystalline forms may be prepared according to the following general procedures.

Typical abbreviations used are outlined below.

| | |
|---|---|
| Name | Abbreviation |

Solvents

| | |
|---|---|
| n-propanol | n-PrOH |
| Acetonitrile | ACN |
| Dichloromethane | DCM |
| Ethanol | EtOH |
| Ethyl Acetate | EtOAc |
| Methanol | MeOH |
| N,N-Dimethylformamide | DMF |
| tert-Butyl Methyl Ether | MtBE |
| Tetrahydrofuran | THF |
| Trifluoroacetic Acid | TFA |
| Trifluoroethanol | TFE |

Units

| | |
|---|---|
| Celsius | C |
| Degrees | ° |
| Equivalents | eq. |
| Gram | g |
| Hour | hr |
| Kelvin | K |
| Liters | L |
| Milligrams | mg |
| Milliliters | mL |
| Minute | min |
| Second | sec |
| volume | vol. |
| Watt | W |
| weight | wt. |

A Rigaku SmartLab X-Ray Diffractometer was configured in Bragg-Brentano reflection geometry equipped with a beam stop and knife edge to reduce incident beam and air scatter. Typical parameters for XRPD are listed below.

Parameters for Reflection Mode

| | |
|---|---|
| Geometry | Bragg-Brentano |
| Tube Anode | Cu |
| Tube Type | Long Fine Focus |
| Tube Voltage (kV) | 40 |
| Tube Current (mA) | 44 |
| Detector | D/tex Ultra 250 (XR1 or XR3) HyPix-3000 |
| | (XR4) |
| Monochromator | Ni foil Cu Kβ Filter |
| Incident Slit (°) | 1/3 |
| Receiving Slit 1 (mm) | 18 |
| Receiving Slit 2 (mm) | Open |
| Start Angle 2θ (°) | 2 |
| End Angle 2θ (°) | 40 |
| Step Size (°) | 0.02 |
| Scan Speed (°/min) | 6 |
| Spinning (rpm) | 11 |
| Sample Holder | Low-background Si |

Differential scanning calorimetry analyses were carried out using a TA Instruments Q2500 Discovery Series instrument. The instrument temperature calibration was performed using indium. The DSC cell was kept under a nitrogen purge of −50 mL per minute during each analysis. The sample was placed in a standard, crimped, aluminum pan and was heated from approximately 25° C. to 300° C. at a rate of 10° C. per minute.

Thermogravimetric analysis was carried out using a TA Instruments Q5500 Discovery Series instrument. The instrument balance was calibrated using class M weights and the temperature calibration was performed using alumel. The nitrogen purge was −40 mL per minute at the balance and −60 mL per minute at the furnace. Each sample was placed into a pre-tared platinum pan and heated from approximately 25° C. to 300° C. at a rate of 10° C. per minute.

Dynamic Vapor Sorption (DVS) was carried out using a TA Instruments Q5000 Dynamic Vapor Sorption analyzer. The instrument was calibrated with standard weights and a sodium bromide standard for humidity. Approximately 10-25 mg of sample was loaded into a metal-coated quartz pan for analysis. The sample was analyzed at 25° C. with a maximum equilibration time of one hour in 10% relative humidity (RH) steps from 5 to 95% RH (adsorption cycle) and from 95 to 5% RH (desorption cycle). The movement from one step to the next occurred either after satisfying the equilibrium criterion of 0.01% weight change or, if the equilibrium criterion was not met, after one hour. The percent weight change values were calculated using Microsoft Excel®.

High Pressure Liquid Chromatography analyses for samples at pH 1.2 through 3 were carried out on an Agilent HPLC 1260 Infinity II series equipped with a UV-DAD detector. HPLC analyses for samples at pH 4.5 and greater were carried out on an Agilent 1100 series instrument equipped with a UV detector. A reference standard was prepared and injected with every sequence. Prior to HPLC analysis, samples prepared in buffers pH 1.2 through 3 were diluted 1:10 and samples prepared in buffers 4.5 and greater were diluted 1:1. Dilutions were carried out using the corresponding. Typical parameters for HPLC are listed below.

| Column | Waters Sunfire C18, 5 μm, 150 × 4.6 mm P# | | |
|---|---|---|---|
| | 186002554 | | |
| Column Temperature | 30 °C | | |
| Detection | UV at 254 nm | | |
| Injection Volume | 2 μL | | |
| Flow Rate | 1 mL/min | | |
| Mobile Phase A | 0.05% TFA in Water | | |
| Mobile Phase B | 0.05% TFA in ACN | | |
| | Time | % Mobile | % Mobile |
| Gradient | (minutes) | Phase A | Phase B |
| | 0.0 | 90 | 10 |
| | 3.0 | 80 | 20 |
| | 10.0 | 50 | 50 |
| | 13.0 | 5 | 95 |
| | 13.1 | 5 | 95 |
| | 18 | 90 | 10 |
| | 30.0 | 90 | 10 |

| | |
|---|---|
| Run Time | 30 minutes |
| Needle Wash | 0.1% TFA in MeOH:Water (50:50) % v/v |
| Diluent | 0.1% TFA in MeOH:Water (50:50) % v/v |

### Synthesis of Materials

### Compound 1

Compound 1, can be prepared following the procedures described in WO 2019/035865, the entirety of which is incorporated by reference.

### Hemi-Hydrate Hemi-Sulfate Salt Formation (Form A)

Salt formation was conducted by slurrying Compound 1, in n-PrOH/H₂O (8:2 v/v). The slurry was then heated to 80° C. and stirred for 15 minutes. Full dissolution was observed after 15 minutes. Sulfuric acid (98%, 0.55 eq.) was added slowly to the solution dropwise. Spontaneous nucleation occurred in less than 5 minutes. Slurry was then stirred at 80° C. for 1 hour. After, the slurry was cooled to 25° C. over the course of 1 hour. The slurry was filtered, washed 3 times with two volumes of n-PrOH and dried in vacuum oven overnight at 50° C. The slurry was filtered and then analyzed by XRPD.

As discussed in greater detail in the Examples, crystalline Form A of a hemi-hydrate hemi-sulfate salt of Compound 1 was found to have a variety of favorable physicochemical properties, including high crystallinity, stability in multiple solvent systems (e.g. especially containing water), relatively small particle size (e.g. below 20 μm under microscope so as to potentially avoid the subsequent micronization), and stability in humidity (e.g. at least 20% RH or at least a water activity of 0.2), and demonstrate favorable plasma concentration-time profiles and pharmacokinetic parameters.

### Phosphate Salt Formation (Form B)

Salt formation was conducted by adding 451.6 mg of Compound 1 hydrate to 18.1 mL n-PrOH/H₂O (8:2 v/v) in a 20 mL scintillation vial. The vial was heated to 80° C. and 82.4 μL phosphoric acid solution (85%) was added after 15 minutes. A seed crystal of the free base form was added to the vial and the system became a viscous slurry within 1-2 minutes. The system was stirred at 80° C. for 45 minutes and left to cool to room temperature and stir overnight. The slurry was filtered and the wet cake was washed three times with two volumes of n-PrOH. The wet cake was dried in a vacuum oven at 50° C. for 5 hours.

### DL-Tartrate Salt Formation (Form C)

Salt formation was conducted by adding 39.5 mg of Compound 1, 7 mL of 95:5 THF:H₂O, and 15.1 mg of tartaric acid to a 2-dram vial. The slurry was sonicated, resulting in a clear solution. The solution was left at room temperature overnight, and remained clear the next day. The sample was then placed in a refrigerator at 5° C. overnight and remained clear the next day. The sample was then placed in a freezer at −15° C. for 4 days. Crystals were observed to form in this time. The slurry was centrifuged, the mother liquor decanted, and the solids allowed to air dry at room temperature.

### DL-Tartrate Salt Formation (Form F)

DL-Tartrate Salt Form F can be obtained from a slurry of material in a variety of solvents including THF (slurry at ambient temperature), methyl ethyl ketone (slurry at 50 C), isopropyl alcohol and 2-Me THF.

### L-Tartrate Salt Formation (Form I)

To a glass flask was added Compound 1 (1.0 equiv) and EtOH/water (95:5 v/v 177V) and heated to 60° C. to obtain a clear solution. L-tartrate (1.0 equiv.) was added and stirred for 1 hour. The solution was allowed to cool to 40° C. over 2 hours and then stirred for an additional 4 hours. The solution was allowed to cool to Room Temperature over 2 hours and then stirred overnight. The solution was cooled to 5° C. over two hours and stirred overnight. The solution was further cool to −15° C. over two hours and stirred overnight. The resulting slurry was filtered and dried under vacuum at room temperature to obtain an off-white solid (3.0 g: 71.2% yield).

### Hydrochloride Salt Formation (Form D)

Salt formation was conducted by adding 10.6 mg of Compound 1, 0.25 mL of THF, and 3.2 μL of hydrochloric acid (aqueous, 37%). The resulting slurry was centrifuged, the mother liquor decanted, and the solids allowed to air dry at room temperature.

### Hydrochloride Salt Formation (Form D-1)

To a glass flask, was added Compound 1 (1.0 eq.) and THF (24 V) to obtain a slurry. Concentrated HCl (1.42 eq.) was added dropwise and stirred for 3 days at RT (10-20° C.). The resulting slurry was filtered and dried under vacuum at room temperature to obtain off-white solids (1.8 g, yield: 95.6%).

### Hydrochloride Salt Formation (Form D-2)

To a glass flask was added Compound 1 (1.0 eq.) and THF (12.5 V) to obtain a slurry. Concentrated HCl (1.0 eq.) was added dropwise and stirred for 2 days at RT (10-20° C.). The resulting mixture was heated to 50° C. and stirred for 1 day. The resulting slurry was filtered and dried under vacuum at room temperature to obtain off-white solid. The solid was mixed with acetone/H₂O (12.5 V) to obtain a slurry and stirred for 1 day at 45° C. To this solution was added concentrated HCl (1.0 eq.) dropwise at 55° C. and stirred for 2 days. The solid was filtered and dried the solids under vacuum at room temperature to obtain off-white solids (1.58 g, yield: 82.3%).

### Monohydrate Free Base Formation (Form E)

Formation of the monohydrate free base was conducted by wet-milling Compound 1.

In an alterative method for making Form E, Compound 1 (1.0 wt) was suspended in n-PrOH (13 vol) and water (2.5 vol) and then heated to 80C. After dissolution, the mixture was cooled to 65C, seed (0.005 wt) was added, and the suspension was stirred for 2 h. The suspension was cooled to 20C over 3 h and held for 2 h. The suspension was filtered, rinsed with 10:1 heptane:n-PrOH (1 vol) and then with heptane (2 vol), and dried under vacuum at 30C for 20 h.

### Spray Dried Dispersion Formulations

### Form E:

A spray dried dispersion was prepared by dissolving 6 g of Form E and 11 g of polymer hypromellose acetate succinate medium grade (HPMCAS-MG) (35/65 w/w) or polyvinylpyrrolidone/vinyl acetate copolymer (PVPVA) (35/65 w/w) were dissolved in 450 mL of DCM:MeOH (66/34 w/w). The mixture was stirred at 40° C. until homogenous. The solution was filtered and spray dried in a lab scale Buchi spray dryer. The spray dried material was secondarily dried in a vacuum oven at 40° C.

### Compound 1:

A spray dried dispersion of Compound 1 to HPMCAS (15/85) was prepared according to the Table below:

| Parameter | Conditions |
|---|---|
| Formulation | 15/85 Compound |
| | 1/HPMCAS-H |
| Target batch size (g) | 67 |
| Solids loading in solution | 3.3 |
| (wt %) | |
| Solvent system | 80/20 THF/Water |
| Atomizer | Pressure swirl: Schlick 2.0 |
| Atomization pressure (psig) | 120 |
| Solution flow (g/min) | 43 |
| Drying gas flow (g/min) | 500 |
| Dryer inlet temperature (°C) | 147 |
| Dryer outlet temperature | 46 |
| (°C) | |
| Dry yield (%) | 89 |
| Secondary drying | Vacuum dried for 22 hours |
| | at 40 °C |

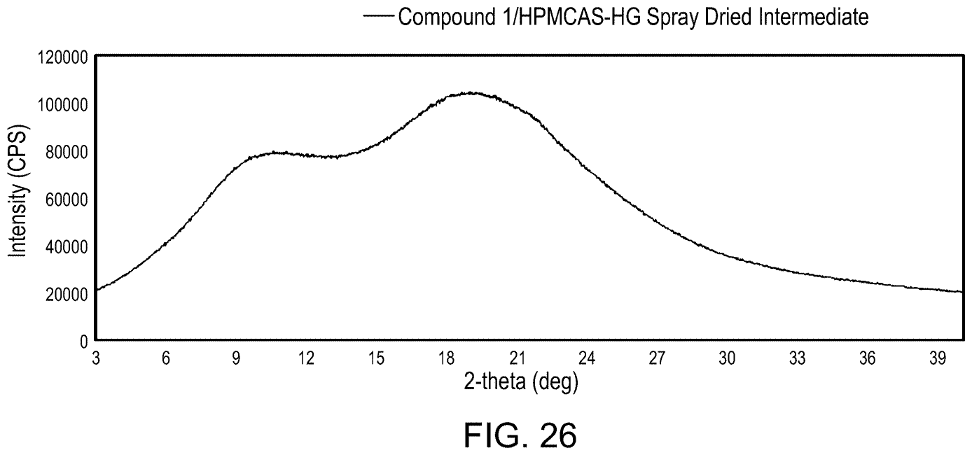

The solution was prepared at 0.5 wt % Compound 1 in solution with 80/20 THF/water, with Form E dissolving completely prior to spray drying. XRPD pattern of the spray dried material is shown in FIG. 26.

### Tablet Formulations:

The tablets described herein (i.e., the tablets comprising the spray dried dispersions of Compound 1; which can be prepared using any one of crystalline hemi-hydrate hemi-sulfate salt Form A; crystalline hemi-hydrate hemi-sulfate salt Form H; crystalline phosphate salt Form B; crystalline DL-tartrate salt form C; crystalline DL-tartrate salt form F; crystalline DL-tartrate salt form G; crystalline L-tartrate salt form I; crystalline hydrochloride salt Form D (i.e., D, D-1, and D-2); crystalline hydrochloride salt Form J; crystalline hydrochloride salt Form K; and crystalline free base monohydrate Form E) can be formed using techniques familiar to those possessing skill in the art. Other tablet formulations comprising crystalline hemi-hydrate hemi-sulfate salt Form A; crystalline hemi-hydrate hemi-sulfate salt Form H; crystalline phosphate salt Form B; crystalline DL-tartrate salt form C; crystalline DL-tartrate salt form C-1, crystalline DL-tartrate salt form F; crystalline DL-tartrate salt form G; crystalline L-tartrate salt form I; crystalline hydrochloride salt Form D (i.e., D, D-1, and D-2); crystalline hydrochloride salt Form J; crystalline hydrochloride salt Form K; and crystalline free base monohydrate Form E can be prepared using techniques familiar to those possessing skill in the art.

### Solubility Experiments

### Sample Preparations

Solubility samples were prepared by weighing about 10 mg of each salt and adding 1 mL of media and vortexed briefly to obtain suspensions. For low pH media (pH 1 and simulated gastric fluid), the solid dissolved instantly and therefore 100 uL increments of the media was added to estimate the solubility. For such instances, the solubility is calculated as greater than (>).

The sample vials were capped and wrapped in aluminum foil, and placed on a shaker at 37° C. The shaker was set at about 100 rpm and vials were removed at specified time points. The vials were observed, and the supernatants were transferred to Eppendorf tubes and centrifuged at 3K for 10 minutes. The clear supernatants were transferred to HPLC vials. The samples were diluted prior to HPLC analysis using the dilution media by 2× or used without dilution. pH was measured of the 24 hour timepoint after centrifugation using a micro pH probe.

### Preparation of Buffer Solutions

pH 2 buffer USP: Mixed 50 mL of 0.2M potassium chloride and 13 mL 0.2M HCl, diluted to 200 mL with water.
pH 4.5 buffer USP: 5.44 g of potassium dihydrogen phosphate was diluted to 1000 mL with water.
pH 6.8 buffer USP: Mixed 50 mL of 0.2M potassium phosphate and 22.4 mL 0.2M sodium hydroxide. Diluted with water to 200 mL.
SGF: 2.0109 g of NaCl was dissolved in water, added 7 mL of conc. HCl and diluted to 1 L.
FeSSIF: 4.0321 g of NaOH and 11.8726 g of NaCl were dissolved in water, added 8.65 mL of glacial acetic acid, adjusted the pH to 5.0 and diluted to 1 L.
FaSSIF: 0.4247 g of NaOH, 3.9548 g of sodium phosphate monobasic monohydrate and 6.1900 g of NaCl were dissolved in water, adjusted the pH to 6.5 and diluted to 1 L.

### pH Solubility

pH solubility was determined in buffers ranging from pH 1 to pH 6.8 at 2 hours and 24 hours. At each time point, the solution was analyzed by HPLC and residue was vacuum dried and analyzed by XRPD. The pH of the solution was measured at 24 hours.

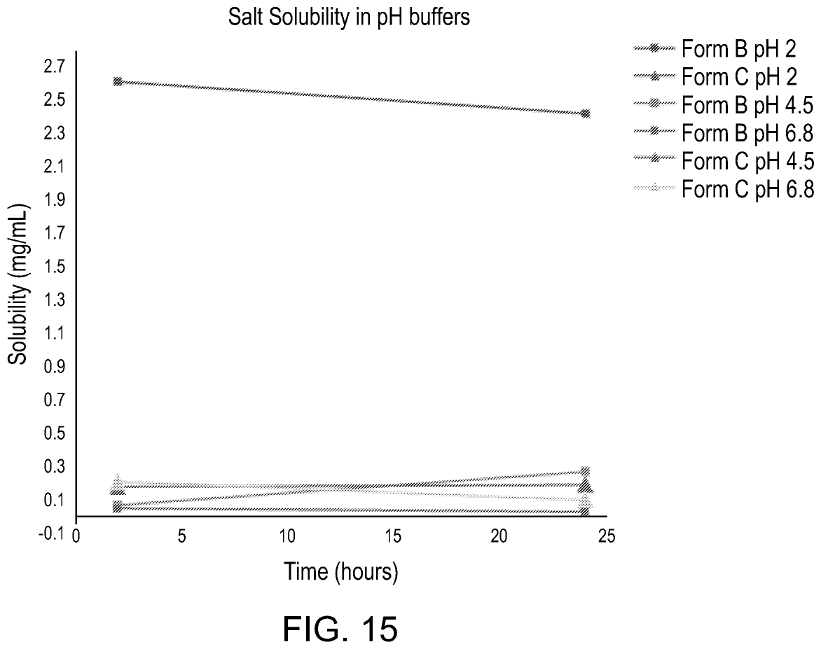

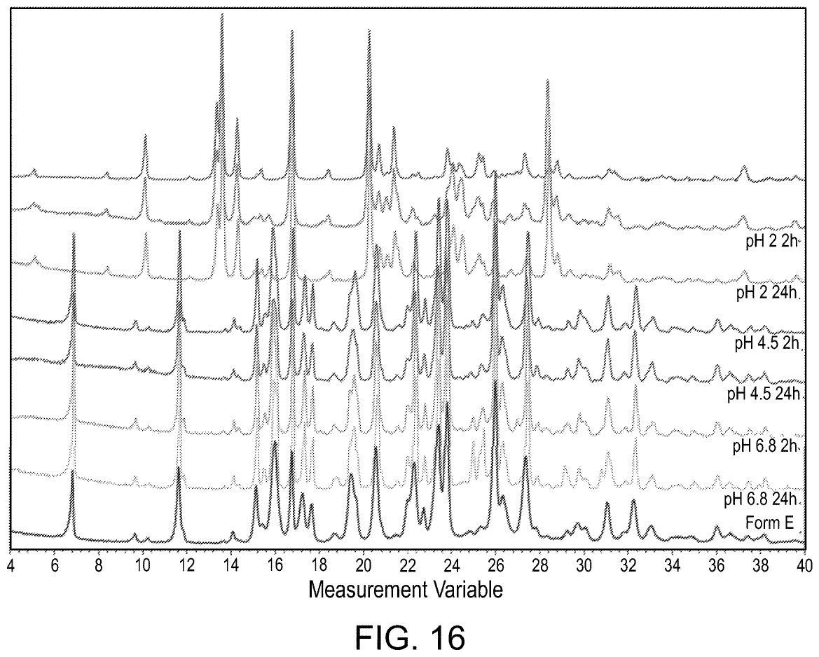

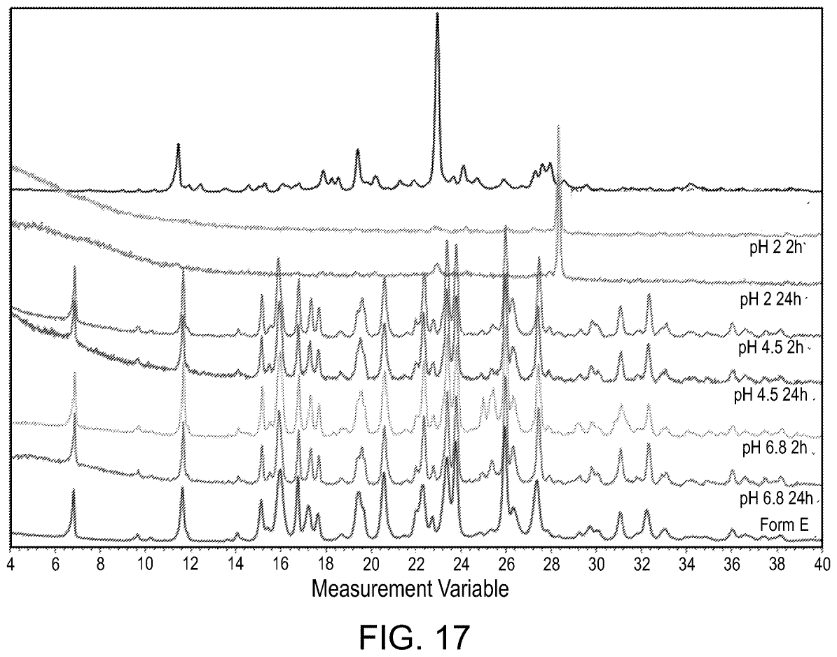

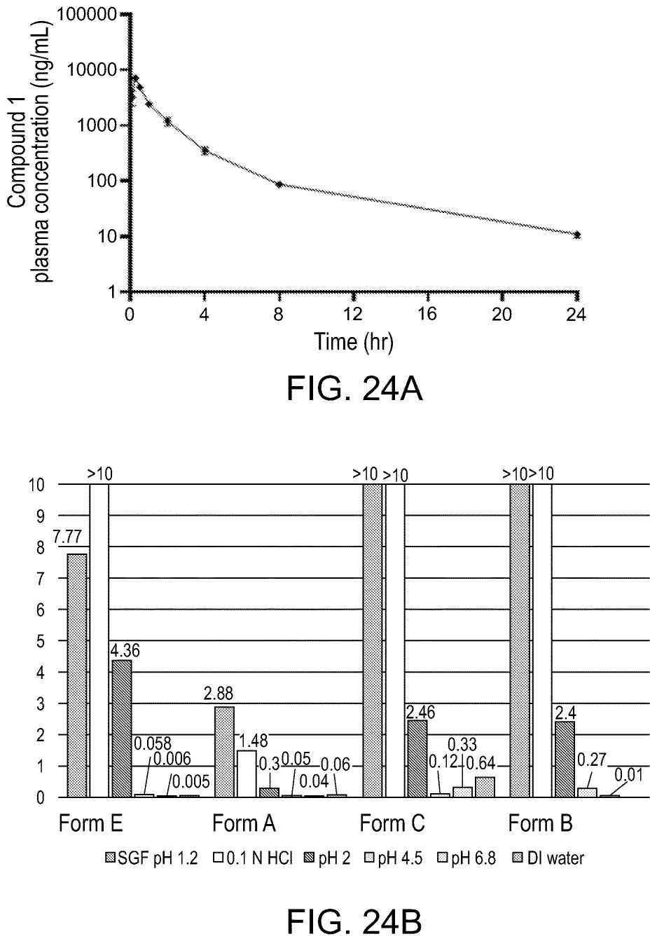

Overall, Form C exhibited highest solubility, with Form B exhibiting similar level of solubility. In 0.1N HCl, both Form B and From C dissolved instantly, and therefore the solubility was estimated by adding 100 uL aliquots to about 10 mg of weighed solid. XRPD analysis showed salt disproportionation at pH 4.5 and higher. These results are summarized in FIGS. 15-17, FIG. 24B, and Tables 1-3 below:

### Table 1: PH Solubility Results for Form B

TABLE 1
pH solubility results for Form B

| | | | |
|---|---|---|---|
| Media | Time | solubility (mg/mL) | pH (24 h) |

| 0.1 N HCl | — | | >16.7 mg/mL | 1.44 |
|---|---|---|---|---|
| pH 2 (USP) | 2 | h | 2.59 | 2.26 |
| | 24 | h | 2.40 | |
| pH 4.5 (USP | 2 | h | 0.07 | 4.01 |
| | 24 | h | 0.27 | |
| pH 6.8 (USP) | 2 | h | 0.03 | 5.09 |
| | 24 | h | 0.01 | |

### Table 2. pH Solubility Results for Form C

TABLE 2
pH solubility results for Form C

| | | | |
|---|---|---|---|
| Media | Time | solubility (mg/mL) | pH (24 h) |

| 0.1 N HCl | — | | >10.89 mg/mL | 1.27 |
|---|---|---|---|---|
| pH 2 (USP | 2 | h | 3.81 | 2.52 |
| | 24 | h | 2.46 | |
| pH 4.5 (USP) | 2 | h | 0.15 | 3.92 |
| | 24 | h | 0.12 | |
| pH 6.8 (USP) | 2 | h | 0.37 | 3.54 |
| | 24 | h | 0.33 | |

### Table 3. pH Solubility Results for Form A

TABLE 3
pH solubility results for Form A

| | | | |
|---|---|---|---|
| Media | Time | solubility (mg/mL) | pH (24 h) |

| | | | | |
|---|---|---|---|---|
| 0.1 N HCl | 2 | h | 1.97 | — |
| | 24 | h | 1.48 | |
| pH 2 (USP) | 2 | h | 0.40 | 2.06 |
| | 24 | h | 0.30 | |
| pH 4.5 (USP) | 2 | h | 0.06 | 4.32 |
| | 24 | h | 0.05 | |
| pH 6.8 (USP) | 2 | h | 0.04 | 5.63 |
| | 24 | h | 0.07 | |

### Kinetic Solubility

Kinetic solubility was determined in biorelevant media. At each time point, the solution was analyzed by HPLC. The solid residue was analyzed by XRPD at selected time points. The pH of the solution was measured at 24 hours.

Overall, Form C exhibited highest solubility in all media tested. In simulated gastric fluid (SGF), both Form B and Form C dissolved instantly, and therefore the solubility was evaluated by adding 100 uL aliquots to about 10 mg of weighed solid. The solubility in the table is given as greater than “>”, calculated based on the total volume of media added to fully dissolve the solid.

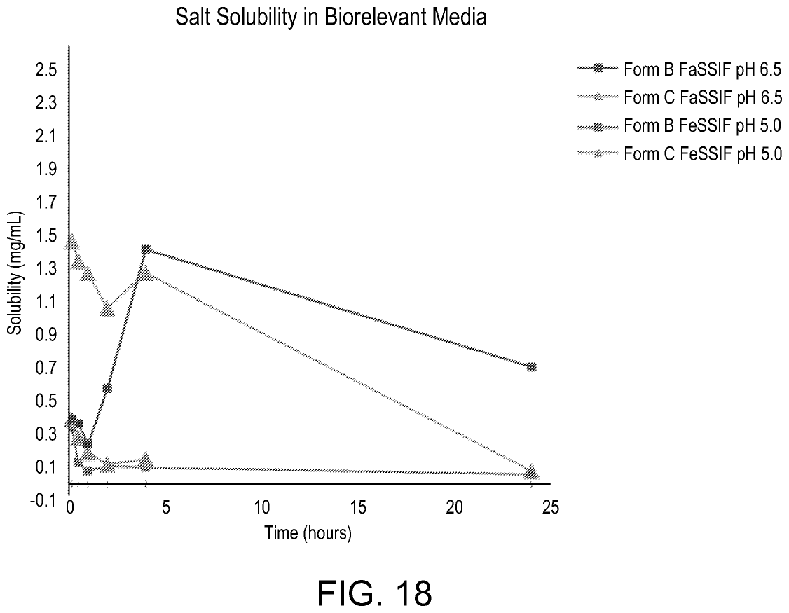

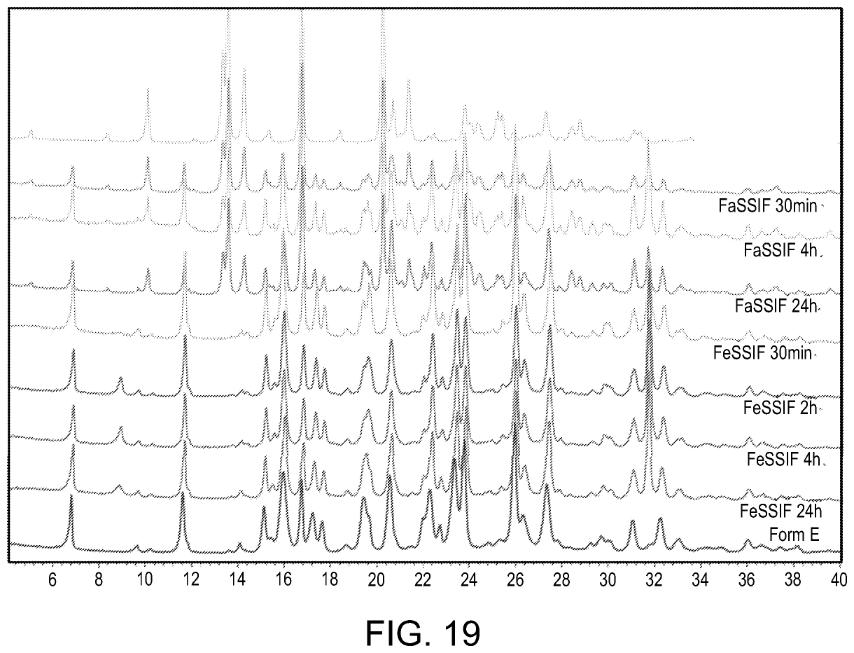

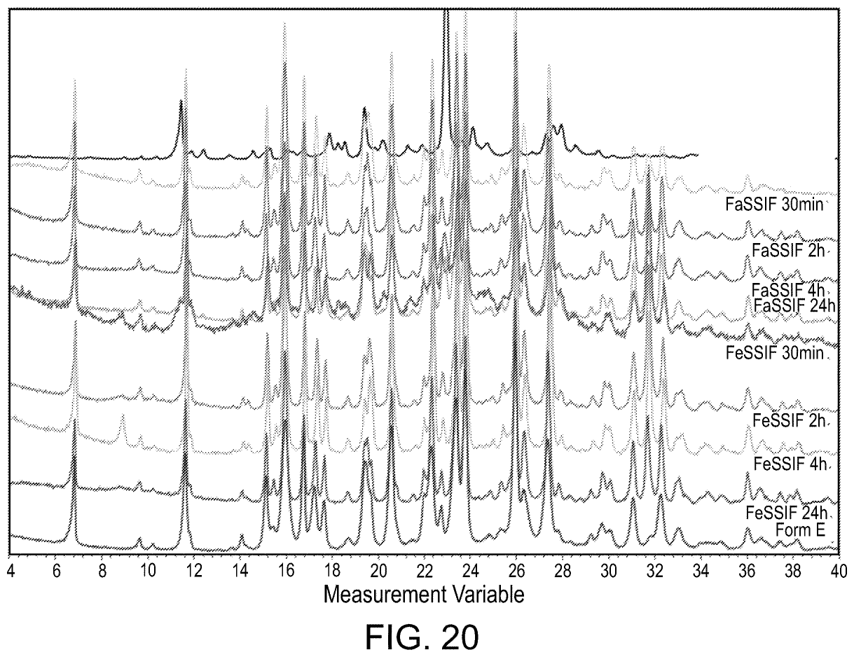

The XRPD analysis results showed salt disproportionation, except for Form B in fasted state simulated intestinal fluid (FaSSIF) where some salt remained. These results are summarized in FIGS. 18-20 and Tables 4-6 below:

### Table 4: Kinetic Solubility Results for Form B

TABLE 4
Kinetic solubility results for Form B

| | | | |
|---|---|---|---|
| Media | Time | solubility (mg/mL) | pH (24 h) |

| SGF | — | | >10 mg/mL | 1.0 |
|---|---|---|---|---|
| pH 1.2 | | | | |
| FaSSIF | 10 | min | 0.38 (suspect) | 3.39 |
| pH 6.5 | 30 | min | 0.36 (suspect) | |
| | 1 | h | 0.24 | |
| | 2 | h | 0.57 | |
| | 4 | h | 1.40 (suspect) | |
| | 24 | h | 0.70 | |
| FeSSIF | 10 | min | 0.34 | 4.79 |
| pH 5.0 | 30 | min | 0.13 | |
| | 1 | h | 0.08 | |
| | 2 | h | 0.11 | |
| | 4 | h | 0.10 | |
| | 24 | h | 0.06 | |

### Table 5: Kinetic Solubility Results for Form C

TABLE 5
Kinetic solubility results for Form C

| | | | |
|---|---|---|---|
| Media | Time | solubility (mg/mL) | pH (24 h) |

| SGF | — | | >11.1 mg/mL | 1.13 |
|---|---|---|---|---|
| pH 1.2 | | | | |
| FaSSIF | 10 | min | 0.65 | 4.60 |
| pH 5.0 | 30 | min | 0.31 | |
| | 1 | h | 0.25 | |
| | 2 | h | 0.20 | |
| | 4 | h | 0.10 | |
| | 24 | h | 0.12 | |
| FeSSIF | 10 | min | 0.53 | 3.35 |
| pH 6.5 | 30 | min | 0.86 | |
| | 1 | h | 0.91 | |
| | 2 | h | 0.99 | |
| | 4 | h | 0.81 | |
| | 24 | h | 0.02 | |

### Table 6: Kinetic Solubility Results for Form A

TABLE 6
Kinetic solubility results for Form A

| | | | |
|---|---|---|---|
| Media | Time | solubility (mg/mL) | pH (24 h) |

| | | | | |
|---|---|---|---|---|
| SGF | 10 | min | 2.85 | 0.87 |
| pH 1.2 | 30 | min | 4.96 | |
| | 1 | h | 2.84 | |
| | 2 | h | 3.24 | |
| | 4 | h | 3.77 | |
| | 24 | h | 2.88 | |
| FaSSIF | 10 | min | 0.12 | 3.91 |
| pH 6.5 | 30 | min | 0.12 | |
| | 1 | h | 0.11 | |
| | 2 | h | 0.16 | |
| | 4 | h | 0.17 | |
| | 24 | h | 0.18 | |
| FeSSIF | 10 | min | 0.55 | 5.74 |
| pH 5.0 | 30 | min | 0.10 | |
| | 1 | h | 0.23 | |
| | 2 | h | 0.07 | |
| | 4 | h | 0.07 | |
| | 24 | h | 0.03 | |

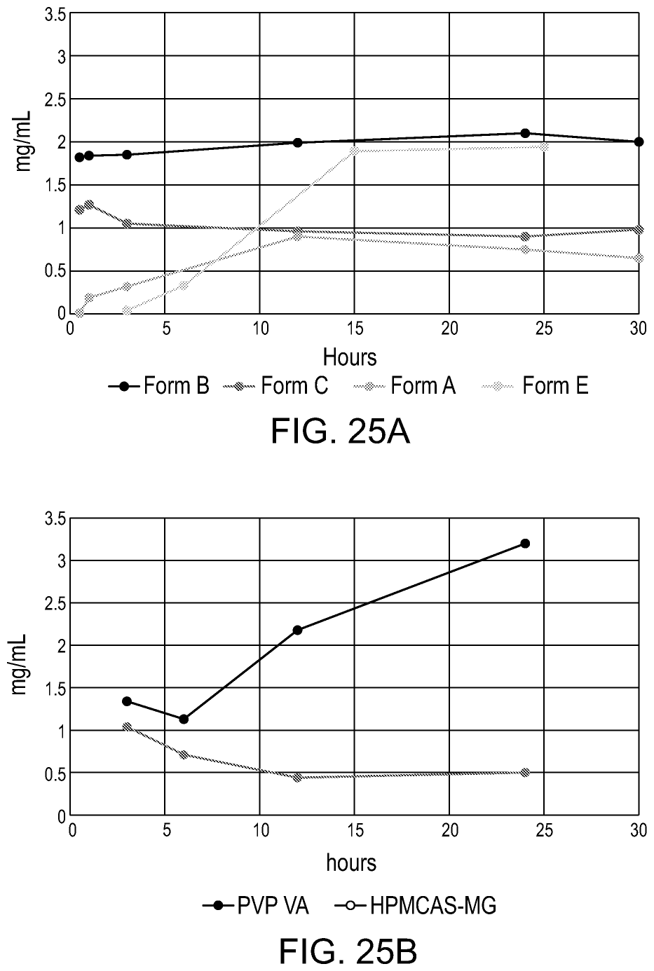

The solubility of 10 mg of Forms A, B, C, and E in a solution of 1 mL of 0.5% methyl cellulose and 0.2% Tween 80 in deionized water are shown in FIG. 25A.

### Pharmacokinetics of Crystalline Forms A, B, C, and E

### Study Design

Twenty-one (21) male Sprague Dawley rats (purchased from Shanghai Laboratory Animal Center Co., Ltd., Shanghai, China) were randomized into 7 groups (3 animals per group). The animals fasted overnight, the forms of Compound 1 shown in Table 7 were administered by oral gavage, and the animals were fed 4 hours post-dose. Each form of Compound 1 was administered in the formulation as shown in Table 7.

### Table 7: Formulations Used in Studies

TABLE 7
Formulations used in studies

| | | | Dose | Dose | Dose |
|---|---|---|---|---|---|
| | Form of | | Level | Conc. | Volume |
| Group | Compound 1 | Formulation | (mg/kg) | (mg/mL) | (mL/kg) |
| 1 | Free Base | 10% HP-B-CD in 50 | 10 | 1 | 10 |
| | Monohydrate Form E | mM citrate buffer | | | |
| | (wet milled) | | | | |
| 2 | Free Base | | 10 | 1 | 10 |
| | monohydrate Form E | | | | |
| | (Unmilled) | | | | |
| 3 | Phosphate Salt | 0.2% Tween 80, | 10 | 1 | 10 |
| | Form B | 0.5% | | | |
| 4 | DL-Tartrate Salt | microcrystalline | 10 | 1 | 10 |
| | Form C | cellulose | | | |
| 5 | Hemi-hydrate Hemi | | 10 | 1 | 10 |
| | Sulfate Salt | | | | |
| | Form A | | | | |
| 6 | SDD with PVPVA | 1% w/w HPMCAS- | 10 | 1 | 10 |
| | and 35% drug | MF, 1% w/w PVP | | | |
| | loading | K30, 2% w/w TPGS, | | | |
| 7 | SDD with HPMC-AS | 0.1% w/w | 10 | 1 | 10 |
| | and 35% drug loading | Simethicone | | | |

HP-β-CD: Hydroxypropyl-beta-cyclodextrin
HPMCAS-MF: Hydroxypropylmethylcellulose acetate succinate
PVP K30: Polyvinylpyrrolidone
TPGS: D-alpha Tocopheryl polyethylene glycol succinate

### Blood Collection

Blood was serially collected from each animal at 0 (pre-dose), 0.083, 0.25, 0.5, 1, 2, 4, 8, and 24 h post-dose. For each collection, the animal was restrained manually, and approximately 150 μL of blood sample was collected via the tail vein into K₂EDTA tubes. Plasma samples were stored at approximately −70° C. until analysis.

To enable the calculation of absolute bioavailability, Compound 1 was formulated as a solution in 10% N-methyl-2-pyrrolidone, 10% Solutol HS 15, and 80% saline and administered at 1 mg/kg as intravenous (IV) bolus dose to a separate group of male Sprague Dawley rats, and blood samples were collected at similar timepoints to those shown above. Further, plasma samples were obtained from the collected samples and stored at approximately −70° C. until analysis.

### Sample Preparation and Analysis

The concentration of Compound 1 in the plasma samples was determined by LC-MS/MS analysis.

A 20 μL aliquot of each plasma sample was mixed with 200 μL of acetonitrile containing glipizide as an internal standard (40 ng/mL). The resulting mixture was vortexed at 1500 rpm for 2 min and centrifuged at 5800 rpm for 10 min. A 1 μL sample from the supernatant was injected into LC-MS/MS.

LC-MS/MS analysis was conducted on a UPLC/MS-MS (API 6500+) system under the conditions outlined in Table 8

### Table 8: Conditions for LC-MS/MS Analysis

TABLE 8
conditions for LC-MS/MS analysis

| | |
|---|---|
| Column | ACQUITY UPLC BEH C18 (2.1 × 50 mm, 1.7 μM) |
| Mobile phase A | Water containing 0.1% Formic Acid |
| Mobile phase B | Methanol containing 0.1% Formic Acid |
| Flow rate | 0.6 mL/min |
| Analysis time | 2 min |
| Gradient | Analysis was conducted over a 2-min gradient elution |
| Program | program employing Mobile Phase A (98-5%) and |
| | Mobile Phase B (2-95%) |

### Results

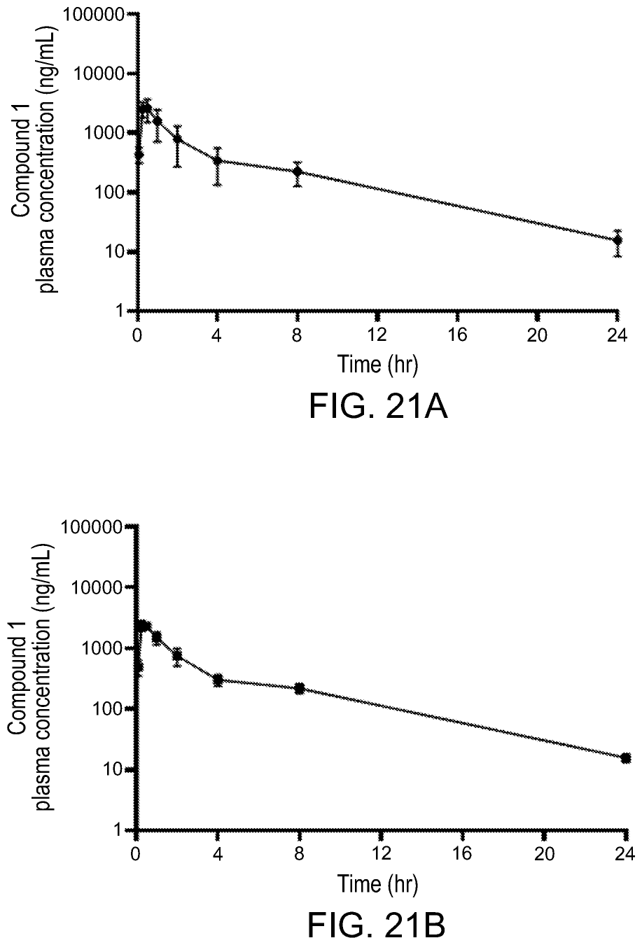

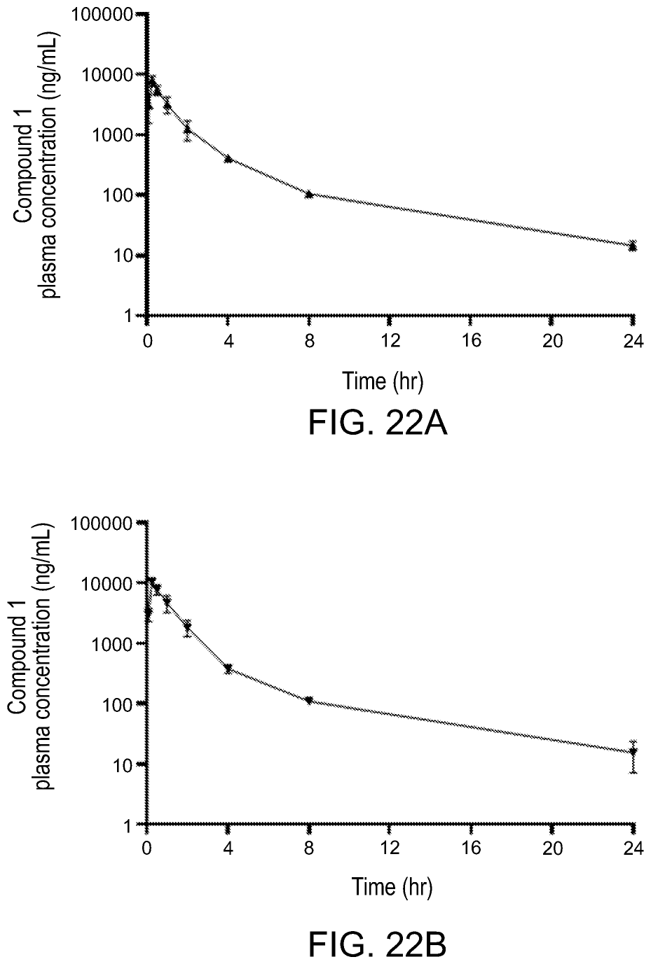

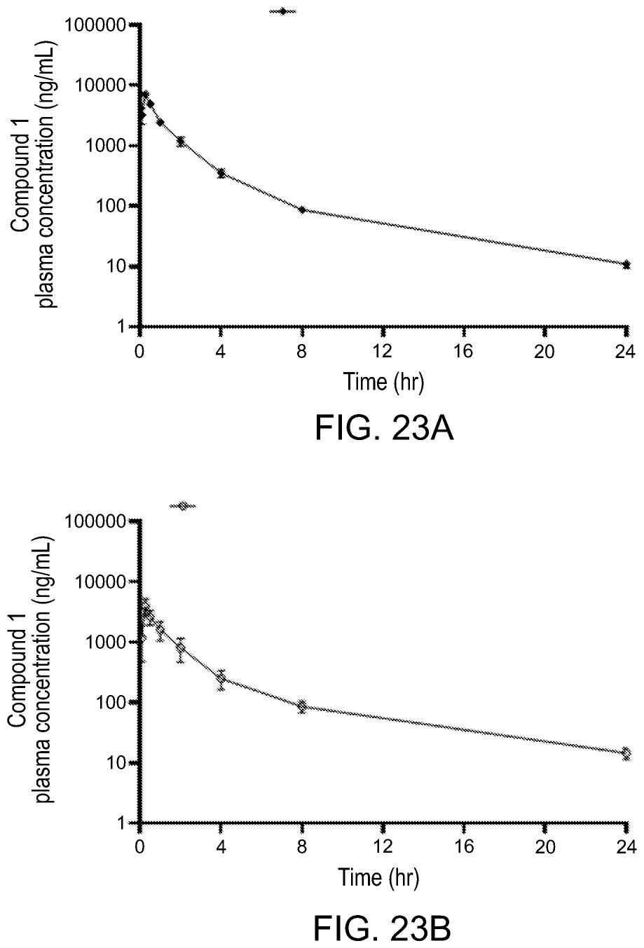

The mean plasma concentration-time profiles of Compound 1 in each of the 7 study groups are shown in FIGS. 21A-24A. The AUCinf and absolute bioavailability (% F) of each form of Compound 1 are reported in Table 9.

Historical data for the IV administration of Compound 1 is provided in Table 9. The absolute bioavailability of each form of Compound 1 was determined by dividing the AUCinf of the form in question by the AUCinf obtained historical data by IV administration of Compound 1. The measured absolute bioavailability of the phosphate salt, DL-tartrate, and hemi-hydrate hemi sulfate salt, as reported in Table 9, was higher than the measured bioavailability of the other forms of Compound 1.

### Table 9. Absolute Bioavailability of Compound 1 Solid Forms in Male Sprague Dawley Rats

TABLE 9
Absolute Bioavailability of Compound 1
Solid Forms in male Sprague Dawley rats

| | | Dose | AUC_{inf} | |
|---|---|---|---|---|
| | | Level | (ng · | Absolute |
| Group | Form of Compound 1 | (mg/kg) | hr/mL) | % F |
| | IV administration (historical) | 1 | 1560 | 100 |
| 1 | Free Base Hydrate Form E (wet | 10 | 7378 | 47 |
| | milled) | | | |
| 2 | Free Base Hydrate Form E | 10 | 6959 | 45 |
| | (Unmilled) | | | |
| 3 | Form B | 10 | 10846 | 70 |
| 4 | Form C | 10 | 14205 | 91 |
| 5 | Form A | 10 | 9426 | 60 |
| 6 | SDD with PVPVA and 35% | 10 | 6214 | 40 |
| | drug loading | | | |
| 7 | SDD with HPMC-AS and 35% | 10 | 8163 | 52 |
| | drug loading | | | |

### Stability Study

No degradation was observed in the stability study of Form A in the following three conditions: one month under 25±2° C./60±5% RH, one month under 40±2° C./75±5% RH, and one month at 40±2° C./75±5% RH

No degradation was observed for the spray dried dispersion formulation in a solution of 1% w/w hypromellose acetate succinate medium grade; 1% w/w polyvinylpyrrolidone; 2% w/w tocofersolan; and 0.1% Simethicone up to 24 hours. See FIG. 25B.

### Characterization Summary

### Form A

The XRPD for Form A is shown by FIG. 1 and the peak listings are shown in Table 10.

### Table 10

TABLE 10

| Peak Position (°2θ) | Relative Intensity |
|---|---|
| 4.54 | 3 |
| 5.66 | 4 |
| 7.76 | 2 |
| 9.18 | 4 |
| 9.76 | 100 |
| 10.98 | 2 |
| 11.34 | 17 |
| 11.86 | 1 |
| 13.30 | 4 |
| 13.56 | 55 |
| 13.76 | 7 |
| 14.22 | 4 |
| 15.28 | 3 |
| 15.64 | 4 |
| 16.10 | 2 |
| 17.00 | 3 |
| 17.30 | 6 |
| 17.54 | 4 |
| 18.12 | 4 |
| 18.40 | 74 |
| 18.56 | 7 |
| 18.70 | 11 |
| 19.22 | 2 |
| 19.48 | 10 |
| 19.66 | 6 |
| 20.00 | 15 |
| 21.06 | 16 |
| 21.06 | 16 |
| 22.08 | 7 |
| 22.78 | 15 |
| 23.06 | 3 |
| 23.30 | 21 |
| 23.90 | 10 |
| 24.38 | 6 |
| 24.58 | 4 |
| 24.90 | 19 |
| 25.28 | 5 |
| 25.52 | 6 |
| 26.20 | 7 |
| 27.06 | 3 |
| 27.30 | 13 |
| 27.74 | 13 |
| 27.96 | 4 |
| 28.36 | 8 |
| 28.56 | 13 |
| 29.20 | 2 |
| 29.54 | 4 |
| 29.94 | 3 |
| 30.26 | 5 |
| 31.56 | 3 |
| 32.64 | 7 |
| 33.04 | 2 |
| 33.36 | 2 |
| 33.92 | 3 |
| 35.60 | 3 |
| 36.72 | 2 |
| 37.00 | 4 |
| 37.64 | 2 |
| 37.88 | 7 |
| 38.46 | 3 |

TGA of Form A showed about 2.1% weight loss up to 120° C. See FIG. 2. Two thermal events were observed in DSC thermogram with the first peak at 117.3° C. and the second peak at 270.3° C. See FIG. 2. Dynamic vapor sorption of salt Form A at 25° C. showed that the solid picks up about 0.08% moisture from 5% to 95% relative humidity. See FIG. 3. Form A shows about 2% water content via Karl Fischer analysis.

### Form B

The XRPD for Form B is shown by FIG. 4 and the peak listings are shown in Table 11.

### Table 11

TABLE 11

| Angle, 2-θ | d spacing (A°) | Rel. Int. |
|---|---|---|
| 5.09 | 17.331 | 23 |
| 8.39 | 10.526 | 7 |
| 10.15 | 8.710 | 85 |
| 13.37 | 6.616 | 34 |
| 13.61 | 6.502 | 86 |
| 14.30 | 6.188 | 35 |
| 15.24 | 5.808 | 5 |
| 15.37 | 5.761 | 7 |
| 16.79 | 5.277 | 94 |
| 18.45 | 4.805 | 8 |
| 20.29 | 4.373 | 100 |
| 20.73 | 4.281 | 12 |
| 21.41 | 4.147 | 47 |
| 23.87 | 3.725 | 22 |
| 24.40 | 3.646 | 9 |
| 25.28 | 3.520 | 10 |
| 25.47 | 3.495 | 19 |
| 27.38 | 3.254 | 5 |
| 28.85 | 3.092 | 9 |

TGA of Form B showed about 0.3% weight loss up to 125° C. See FIG. 5. One thermal event was observed in the DSC thermogram at about 247.0° C. See FIG. 5. Dynamic vapor sorption of salt Form B at 25° C. showed that the solid picks up about 0.9% moisture from 5% to 95% relative humidity. See FIG. 6. Form B shows about 4.4% water content via Karl Fischer analysis.

### Form C

The XRPD for Form C is shown by FIG. 7 and the peak listings are shown in Table 12.

### Table 12

TABLE 12

| Angle, 2-θ | d spacing (A°) | Rel. Int. |
|---|---|---|
| 8.13 | 10.87 | 42 |
| 10.30 | 8.58 | 5 |
| 12.22 | 7.24 | 7 |
| 12.56 | 7.04 | 6 |
| 13.32 | 6.64 | 61 |
| 13.78 | 6.42 | 6 |
| 14.97 | 5.91 | 64 |
| 16.32 | 5.43 | 10 |
| 16.68 | 5.31 | 8 |
| 17.24 | 5.14 | 6 |
| 17.55 | 5.05 | 23 |
| 18.18 | 4.88 | 8 |
| 19.47 | 4.56 | 7 |
| 20.43 | 4.34 | 78 |
| 20.60 | 4.31 | 100 |
| 21.09 | 4.21 | 7 |
| 21.42 | 4.15 | 18 |
| 22.34 | 3.98 | 18 |
| 22.56 | 3.94 | 39 |
| 23.06 | 3.85 | 10 |
| 24.12 | 3.69 | 9 |
| 24.44 | 3.64 | 7 |
| 24.63 | 3.61 | 25 |
| 25.28 | 3.52 | 65 |
| 25.62 | 3.47 | 29 |
| 26.60 | 3.35 | 8 |
| 28.00 | 3.18 | 5 |
| 28.49 | 3.13 | 6 |
| 30.16 | 2.96 | 5 |
| 31.81 | 2.81 | 5 |
| 32.97 | 2.71 | 7 |
| 33.91 | 2.64 | 6 |
| 34.10 | 2.63 | 5 |

TGA of Form C showed about 18.2% weight loss up to 270° C. See FIG. 8. One thermal event was observed in the DSC thermogram at about 224.2° C. See FIG. 8. Dynamic vapor sorption of salt Form C at 25° C. showed that the solid picks up about 0.06% moisture from 5% to 95% relative humidity. See FIG. 9. Form C shows about 0.11% water content via Karl Fischer analysis.

### Form D

The XRPD for Form D is shown by FIG. 10 and the peak listings are shown in Table 13.

### Table 13

TABLE 13

| Angle, 2-θ | d spacing (A°) | Rel. Int. |
|---|---|---|
| 9.47 | 9.33 | 21 |
| 10.65 | 8.30 | 66 |
| 13.58 | 6.51 | 14 |
| 14.43 | 6.13 | 65 |
| 17.18 | 5.16 | 11 |
| 18.02 | 4.92 | 5 |
| 18.69 | 4.74 | 13 |
| 19.13 | 4.64 | 8 |
| 20.95 | 4.24 | 9 |
| 22.47 | 3.95 | 11 |
| 22.80 | 3.90 | 19 |
| 23.46 | 3.79 | 27 |
| 23.83 | 3.73 | 7 |
| 24.10 | 3.69 | 21 |
| 24.62 | 3.61 | 44 |
| 24.84 | 3.58 | 41 |
| 25.19 | 3.53 | 100 |
| 25.79 | 3.45 | 5 |
| 26.14 | 3.41 | 12 |
| 26.76 | 3.33 | 18 |
| 27.00 | 3.30 | 35 |
| 28.68 | 3.11 | 7 |
| 29.00 | 3.08 | 14 |
| 29.51 | 3.02 | 21 |
| 29.81 | 2.99 | 13 |
| 30.13 | 2.96 | 21 |
| 30.54 | 2.92 | 10 |
| 30.82 | 2.90 | 7 |
| 31.08 | 2.88 | 5 |
| 31.56 | 2.83 | 9 |
| 32.55 | 2.75 | 9 |
| 37.43 | 2.40 | 7 |

TGA of Form D showed about 8.4% weight loss up to 180° C. See FIG. 11. Two thermal events were observed in the DSC thermogram the first at about 120.6° C. and the second at about 190.32° C. See FIG. 11. Form D shows about 2.4% to about 7.4% water content via Karl Fischer analysis.

### Form E

The XRPD for Form E is shown by FIG. 12 and the peak listings are shown in Table 14.

### Table 14

TABLE 14

| Angle, 2-θ | d spacing (A°) | Rel. Int. |
|---|---|---|
| 6.82 | 12.96 | 43 |
| 9.65 | 9.16 | 6 |
| 11.62 | 7.61 | 48 |
| 14.08 | 6.29 | 8 |
| 15.13 | 5.85 | 36 |
| 15.43 | 5.74 | 11 |
| 15.96 | 5.55 | 64 |
| 16.75 | 5.29 | 58 |
| 17.22 | 5.14 | 31 |
| 17.64 | 5.02 | 24 |
| 18.67 | 4.75 | 5 |
| 19.44 | 4.56 | 42 |
| 19.44 | 4.56 | 42 |
| 20.56 | 4.32 | 59 |
| 22.00 | 4.04 | 25 |
| 22.32 | 3.98 | 48 |
| 22.74 | 3.91 | 18 |
| 23.40 | 3.80 | 71 |
| 23.78 | 3.74 | 86 |
| 25.36 | 3.51 | 6 |
| 25.95 | 3.43 | 100 |
| 26.30 | 3.39 | 27 |
| 27.36 | 3.26 | 52 |
| 27.82 | 3.20 | 7 |
| 29.24 | 3.05 | 6 |
| 29.71 | 3.00 | 11 |
| 30.02 | 2.97 | 8 |
| 31.04 | 2.88 | 24 |
| 31.77 | 2.81 | 5 |
| 32.25 | 2.77 | 26 |
| 33.03 | 2.71 | 9 |
| 36.03 | 2.49 | 10 |
| 36.60 | 2.45 | 5 |
| 38.17 | 2.36 | 5 |

TGA of Form E showed about 4.4% weight loss up to 100° C. See FIG. 13. Four thermal events were observed in the DSC thermogram the first at about 112.3° C., the second at about 156.1° C., the third at about 160.4° C., and the fourth at 219.6° C. See FIG. 13. Dynamic vapor sorption of salt Form E at 25° C. showed that the solid picks up about 1.3% moisture from 5% to 95% relative humidity. See FIG. 14. Form E shows about 5.7% water content via Karl Fischer analysis.

### Form (D-1)

The XRPD for Form (D-1) is shown by FIG. 27 and the peak listings are shown in Table 15.

### Table 15

TABLE 15

| Angle, 2-θ | Rel. Int. |
|---|---|
| 8.1 | 12 |
| 9.5 | 37 |
| 10.7 | 83 |
| 14.5 | 62 |
| 15.0 | 35 |
| 15.6 | 35 |
| 21.0 | 36 |
| 24.4 | 55 |
| 25.2 | 100 |

TGA of Form D-1 showed about 2.1% weight loss up to 115° C., about 5.2% weight loss up to 206° C., and about 7.4% weight loss up to 290° C. See FIG. 28. Four thermal events were observed in the DSC thermogram the first at about 194.4° C., the second at about 208.9° C., the third at about 235.8° C., and the fourth at 276.6° C. See FIG. 29. Dynamic vapor sorption of salt Form D-1 at 25° C. showed that the solid picks up about 2.8% moisture from 5% to 95% relative humidity. See FIG. 30. Form D-1 shows about 1.5% water content via Karl Fischer analysis.

### Form D-2

The XRPD for Form D-2 is shown by FIG. 31 and the peak listings are shown in Table 16.

### Table 16

TABLE 16

| Angle, 2-θ | Rel. Int. |
|---|---|
| 8.1 | 10 |
| 9.5 | 9 |
| 10.7 | 100 |
| 14.5 | 60 |
| 24.2 | 13 |
| 25.2 | 11 |

TGA of Form D-2 showed about 2.2% weight loss up to 138° C., about 7.5% weight loss up to 206° C., and about 7.1% weight loss up to 290° C. See FIG. 32. Two thermal events were observed in the DSC thermogram the first at about 208.6° C. and the second at about 277.8° C. See FIG. 33. Dynamic vapor sorption of salt Form D-2 at 25° C. showed that the solid picks up about 1.1% moisture from 2% to 95% relative humidity. See FIG. 34. Form G shows about 7.36% water content via Karl Fischer analysis.

### Form I

The XRPD for Form I is shown by FIG. 35 and the peak listings are shown in Table 17.

### Table 17

TABLE 17

| Angle, 2-θ | Rel. Int. |
|---|---|
| 8.0 | 100 |
| 10.8 | 53 |
| 13.8 | 40 |
| 15.0 | 36 |
| 16.0 | 35 |
| 16.7 | 40 |
| 25.2 | 56 |

TGA of Form I showed about 4.3% weight loss up to 141° C. and about 19.4% weight loss up to 297° C. See FIG. 36. Two thermal events were observed in the DSC thermogram the first at about 65.7° C. and the second at about 180.7° C. See FIG. 37. Dynamic vapor sorption of salt Form I at 25° C. showed that the solid picks up about 10.5% moisture from 2% to 95% relative humidity. See FIG. 38. Form I shows about 1.1% to about 3.3% water content via Karl Fischer analysis.

### Form H

The XRPD for Form H is shown by FIG. 39 and the peak listings are shown in Table 18.

### Table 18

TABLE 18

| Angle, 2-θ | Rel. Int. |
|---|---|
| 4.7 | 37 |
| 6.9 | 100 |
| 7.9 | 37 |
| 10.1 | 67 |
| 11.5 | 16 |
| 12.1 | 20 |
| 14.9 | 39 |
| 15.6 | 35 |
| 16.6 | 62 |
| 17.2 | 33 |
| 17.6 | 37 |
| 19.2 | 50 |
| 19.7 | 66 |
| 21.3 | 11 |
| 22.0 | 23 |
| 23.1 | 41 |
| 23.4 | 32 |
| 24.1 | 24 |
| 24.5 | 74 |
| 25.1 | 53 |
| 25.3 | 63 |
| 26.5 | 87 |
| 27.6 | 40 |
| 28.2 | 46 |
| 29.1 | 15 |
| 30.0 | 14 |
| 30.5 | 15 |
| 33.0 | 26 |

TGA of Form H showed a weight loss of 4.2±0.5% up to 100° C.±2° C. and 7.7±0.5% from 200° C.±2° C. See FIG. 41. Two thermal events were observed in DSC thermogram the first at about 126° C. and the second at about 206° C. See FIG. 40.

### Form F

The XRPD for Form F is shown by FIG. 42 and the peak listings are shown in Table 19.

### Table 19

TABLE 19

| Angle, 2-θ | d-spacing [Å] | Height [cts] |
|---|---|---|
| 4.803 | 18.39879 | 95.38 |
| 9.6415 | 9.17359 | 149.53 |
| 10.3254 | 8.56748 | 69.04 |
| 11.3339 | 7.80728 | 298.9 |
| 12.3273 | 7.18029 | 75.35 |
| 14.5135 | 6.10326 | 99.62 |
| 15.1267 | 5.85719 | 28.41 |
| 16.255 | 5.45308 | 36.77 |
| 16.707 | 5.30655 | 46.62 |
| 17.7704 | 4.99133 | 84.85 |
| 18.2069 | 4.87264 | 109.55 |
| 18.4813 | 4.80091 | 87.18 |
| 19.3366 | 4.59043 | 270.95 |
| 19.8434 | 4.47434 | 33.12 |
| 20.2015 | 4.39582 | 179.95 |
| 21.2997 | 4.17158 | 36 |
| 21.8275 | 4.07191 | 85.94 |
| 22.8402 | 3.89359 | 560.83 |
| 23.9822 | 3.71071 | 90.07 |
| 27.5361 | 3.23934 | 90.95 |

TGA of Form F showed a weight loss of 0.03±0.01% up to 200° C.±2° C. See FIG. 43. One thermal event was observed in DSC thermogram at about 217° C. See FIG. 43.

### Form G

The XRPD for Form G is shown by FIG. 44 and the peak listings are shown in Table 20.

### Table 20

TABLE 20

| Angle, 2-θ | d-spacing [Å] | Height [cts] |
|---|---|---|
| 5.705 | 15.49164 | 69.63 |
| 7.1884 | 12.29776 | 237.91 |
| 7.7306 | 11.43636 | 622.59 |
| 11.2797 | 7.84471 | 49.5 |
| 16.7522 | 5.29234 | 86.6 |
| 18.3075 | 4.84609 | 146.87 |
| 20.0557 | 4.42745 | 13.25 |

TGA of Form G showed a weight loss of 3.428±0.01% up to 110° C.±2° C. and a weight loss of 2.797±0.01% up to 170° C.±2° C. See FIG. 45. Three thermal events were observed in DSC thermogram the first at about 63° C., the second at about 138° C., and the third at about 202° C. See FIG. 45.

### Form J

The XRPD for Form J is shown by FIG. 46 and the peak listings are shown in Table 21.

### Table 21

TABLE 21

| Angle, 2-θ | d-spacing [Å] | Height [cts] | Rel. Int. [%] |
|---|---|---|---|
| 5.1321 | 17.2053 | 749.64 | 31.06 |
| 7.7804 | 11.35387 | 60.03 | 2.49 |
| 8.9391 | 9.88461 | 40.9 | 1.69 |
| 10.6589 | 8.29325 | 323.1 | 13.39 |
| 12.6731 | 6.97933 | 49.06 | 2.03 |
| 14.7212 | 6.01263 | 151.99 | 6.3 |
| 14.9518 | 5.9204 | 1286.21 | 53.29 |
| 15.6107 | 5.67196 | 2099.53 | 86.99 |
| 16.6506 | 5.32 | 612.95 | 25.4 |
| 17.165 | 5.16171 | 64.25 | 2.66 |
| 18.0128 | 4.92063 | 411.49 | 17.05 |
| 19.2007 | 4.61879 | 141.85 | 5.88 |
| 19.7146 | 4.49954 | 449.09 | 18.61 |
| 20.4175 | 4.3462 | 172.84 | 7.16 |
| 20.5818 | 4.31188 | 2010.62 | 83.31 |
| 20.7 | 4.28751 | 103.13 | 4.27 |
| 21.0716 | 4.21274 | 1415.23 | 58.64 |
| 21.4472 | 4.13981 | 467.11 | 19.35 |
| 21.6853 | 4.09489 | 385.94 | 15.99 |
| 21.855 | 4.06346 | 222.4 | 9.21 |
| 22.004 | 4.0363 | 278.02 | 11.52 |
| 22.7809 | 3.90037 | 394.74 | 16.36 |
| 22.931 | 3.87517 | 126.5 | 5.24 |
| 24.1411 | 3.68359 | 2413.45 | 100 |
| 24.2881 | 3.66164 | 1171.44 | 48.54 |
| 24.7069 | 3.60051 | 1918.53 | 79.49 |
| 25.394 | 3.50462 | 444.69 | 18.43 |
| 25.7437 | 3.45781 | 404.09 | 16.74 |
| 26.3294 | 3.38219 | 273.95 | 11.35 |
| 27.1802 | 3.27823 | 137.82 | 5.71 |
| 27.4242 | 3.24961 | 340.23 | 14.1 |
| 28.8252 | 3.09478 | 196.92 | 8.16 |
| 29.2573 | 3.05005 | 186.97 | 7.75 |
| 29.3837 | 3.03721 | 771.96 | 31.99 |
| 29.5099 | 3.02452 | 512.48 | 21.23 |
| 29.6735 | 3.00821 | 108.45 | 4.49 |
| 29.8487 | 2.99095 | 868.33 | 35.98 |
| 30.3475 | 2.94291 | 252.27 | 10.45 |
| 30.7593 | 2.90444 | 96.05 | 3.98 |
| 31.2081 | 2.8637 | 400.86 | 16.61 |
| 32.0865 | 2.78728 | 41.61 | 1.72 |
| 33.397 | 2.68084 | 160.7 | 6.66 |
| 34.4119 | 2.60406 | 111.15 | 4.61 |
| 35.9692 | 2.49481 | 48.32 | 2 |
| 36.5006 | 2.45969 | 32.24 | 1.34 |
| 37.8346 | 2.37598 | 34.21 | 1.42 |
| 39.453 | 2.28216 | 164.04 | 6.8 |

TGA of Form J showed a weight loss of 0.92±0.05% up to 250° C.±2° C. See FIG. 47. Two thermal events were observed in the DSC thermogram the first at about 76° C. and the second at about 229° C. See FIG. 47.

### Form K

The XRPD for Form K is shown by FIG. 48 and the peak listings are shown in Table 22.

### Table 22

TABLE 22

| Angle, 2-θ | d-spacing [Å] | Height [cts] | Rel. Int. [%] |
|---|---|---|---|
| 7.1909 | 12.29341 | 1251.6 | 100 |
| 9.5397 | 9.27128 | 59.6 | 4.76 |
| 10.5594 | 8.37811 | 56.8 | 4.54 |
| 11.9221 | 7.42338 | 15.38 | 1.23 |
| 12.5314 | 7.06379 | 34.07 | 2.72 |
| 13.9578 | 6.34496 | 132.33 | 10.57 |
| 14.3143 | 6.18772 | 54.48 | 4.35 |
| 14.8965 | 5.94717 | 10.26 | 0.82 |
| 15.1958 | 5.8307 | 8.43 | 0.67 |
| 16.3006 | 5.43794 | 31.42 | 2.51 |
| 17.3742 | 5.10425 | 62.99 | 5.03 |
| 19.2591 | 4.60874 | 24.46 | 1.95 |
| 20.1401 | 4.40908 | 54.46 | 4.35 |
| 20.7519 | 4.28045 | 14.12 | 1.13 |
| 22.3103 | 3.98486 | 30.97 | 2.47 |
| 22.9374 | 3.87732 | 25.77 | 2.06 |
| 23.8442 | 3.73187 | 38.36 | 3.07 |
| 25.1572 | 3.54 | 32.13 | 2.57 |
| 25.9736 | 3.43055 | 289.08 | 23.1 |
| 26.9841 | 3.30434 | 231.57 | 18.5 |
| 29.1532 | 3.06324 | 8.39 | 0.67 |
| 30.5785 | 2.92362 | 19.08 | 1.52 |
| 35.9155 | 2.50048 | 7.19 | 0.57 |
| 38.4238 | 2.34283 | 7.21 | 0.58 |
| 39.7718 | 2.26648 | 24.26 | 1.94 |

TGA of Form K showed a weight loss of 10.06±0.05% up to 180° C.±2° C. See FIG. 49. Three thermal events were observed in the DSC thermogram the first at about 55° C., the second at about 234° C., and the third at about 284° C. See FIG. 49.

### Form C-1

The XRPD for Form C-1 is shown by FIG. 50 and the peak listings are shown in Table 23.

### Table 23

TABLE 23

| Angle, 2-θ | Height [cts] |
|---|---|
| 8.174 | 115.309 |
| 12.590 | 38.2786 |
| 13.315 | 263.964 |
| 13.782 | 37.8077 |
| 14.993 | 328.056 |
| 16.356 | 33.3363 |
| 16.667 | 42.7000 |
| 17.234 | 38.5167 |
| 17.553 | 150.485 |
| 18.172 | 51.8405 |
| 19.458 | 41.2461 |
| 20.605 | 738.406 |
| 21.089 | 51.8355 |
| 21.453 | 90.0186 |
| 22.326 | 171.235 |
| 22.557 | 255.012 |
| 23.032 | 94.2370 |
| 23.716 | 30.9270 |
| 24.138 | 53.6594 |
| 24.445 | 65.5132 |
| 24.647 | 126.915 |
| 25.268 | 858.825 |
| 25.597 | 359.693 |
| 26.599 | 82.5584 |
| 27.985 | 31.5329 |
| 28.448 | 29.6709 |
| 31.779 | 51.0699 |
| 32.603 | 17.1365 |
| 36.646 | 44.7609 |
| 37.998 | 23.8395 |

TGA of Form I showed about 17.4% weight loss up to 275° C. See FIG. 52. One thermal event was observed in the DSC thermogram the first at about 218.76° C. See FIG. 52. Dynamic vapor sorption of salt Form. See FIG. 53.

While a number of embodiments have been described, the scope of this disclosure is to be defined by the appended claims, and not by the specific embodiments that have been represented by way of example. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A crystalline form of a hemi-sulfate salt of Compound 1 having the structural formula:

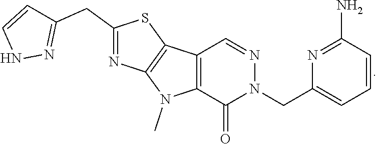 [SMILES: Cn1c2nc(Cc3cc[nH]n3)sc2c2cnn(Cc3cccc(N)n3)c(=O)c21]

2. The crystalline form of claim 1, wherein the hemi-sulfate salt of Compound 1 is a solvate.

3. The crystalline form of claim 1 or 2, wherein the hemi-sulfate salt of Compound 1 is a hydrate.

4. The crystalline form of any one of claims 1-3, wherein the hemi-sulfate salt of Compound 1 is a hemi-hydrate having the structural formula:

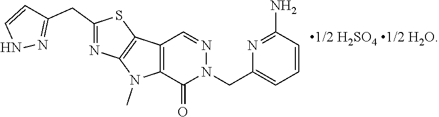 [SMILES: Cn1c2nc(Cc3cc[nH]n3)sc2c2cnn(Cc3cccc(N)n3)c(=O)c21]

5. The crystalline form of any one of claims 1-4, wherein the hemi-sulfate, hemi-hydrate salt of Compound 1 has a moisture content of about 1% to about 3% as measured by Karl-Fischer titration.

6. The crystalline form of any one of claims 1-5, wherein the crystalline form is crystalline Form A characterized by at least two x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°).

7. The crystalline form of any one of claims 1-6, wherein the crystalline form is crystalline Form A characterized by at least three x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°).

8. The crystalline form of any one of claims 1-7, wherein the crystalline form is crystalline Form A characterized by at least four x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°).

9. The crystalline form of any one of claims 1-8, wherein the crystalline form is crystalline Form A characterized by at least five x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°).

10. The crystalline form of any one of claims 1-9, wherein the crystalline form is crystalline Form A characterized by x-ray powder diffraction peaks at 2Θ angles selected from 9.8°(±0.2°), 11.3°(±0.2°), 13.6°(±0.2°), 18.4°(±0.2°), 22.8°(±0.2°), 23.3°(±0.2°), and 28.6°(±0.2°).

11. The crystalline form of any one of claims 1-10, wherein the crystalline form is crystalline Form A characterized by an x-ray powder diffraction pattern substantially the same as depicted in FIG. 1.

12. The crystalline form of any one of claims 1-5, wherein the crystalline form is crystalline Form H characterized by at least two x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°).

13. The crystalline form of any one of claims 1-5 and 12, wherein the crystalline form is crystalline Form H characterized by at least three x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°).

14. The crystalline form of any one of claims 1-5 and 12-13, wherein the crystalline form is crystalline Form H characterized by at least four x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°).

15. The crystalline form of any one of claims 1-5 and 12-14, wherein the crystalline form is crystalline Form H characterized by at least five x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.2°).

16. The crystalline form of any one of claims 1-5 and 12-15, wherein the crystalline form is crystalline Form H characterized by x-ray powder diffraction peaks at 2Θ angles selected from 6.9°(±0.2°), 10.1°(±0.2°), 16.6°(±0.2°), 19.2°(±0.2°), 19.7°(±0.2°), 24.5°(±0.2°), and 26.5°(±0.20).

17. The crystalline form of any one of claims 1-5 and 12-16, wherein the crystalline form is crystalline Form H characterized by an x-ray powder diffraction pattern substantially the same as depicted in FIG. 39.

18. A crystalline form of a phosphate salt of Compound 1 having the structural formula:

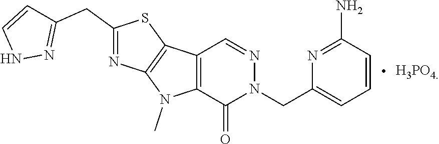

19. The crystalline form of claim 18, wherein the crystalline form is crystalline Form B characterized by at least two x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.20).

20. The crystalline form of claim 18 or 19, wherein the crystalline form is crystalline Form B characterized by at least three x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°).

21. The crystalline form of any one of claims 18-20, wherein the crystalline form is crystalline Form B characterized by at least four x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°).

22. The crystalline form of any one of claims 18-21, wherein the crystalline form is crystalline Form B characterized by at least five x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°).

23. The crystalline form of any one of claims 18-22, wherein the crystalline form is crystalline Form B characterized by at least six x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°).

24. The crystalline form of any one of claims 18-23, wherein the crystalline form is crystalline Form B characterized by x-ray powder diffraction peaks at 2Θ angles selected from 10.2°(±0.2°), 13.4°(±0.2°), 13.6°(±0.2°), 14.3°(±0.2°), 16.8°(±0.2°), 20.3°(±0.2°), and 21.4°(±0.2°).

25. The crystalline form of any one of claims 18-24, wherein the crystalline form is crystalline Form B characterized by an x-ray powder diffraction pattern substantially the same as depicted in FIG. 4.

26. A crystalline form of a tartrate salt of Compound 1 having the structural formula:

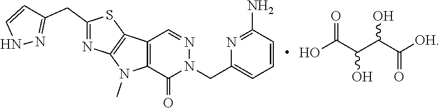 [SMILES: Cn1c2nc(Cc3cc[nH]n3)sc2c2cnn(Cc3cccc(N)n3)c(=O)c21 ; O=C(O)C(O)C(O)C(=O)O]

27. The crystalline form of claim 26, wherein the crystalline form is crystalline Form C characterized by at least two x-ray powder diffraction peaks at 2Θ angles selected from 8.1°(±0.2°), 13.3°(±0.2°), 15.0°(±0.2°), 20.4°(±0.2°), 20.6°(±0.2°), 22.6°(±0.2°), and 25.3°(±0.20).

28. The crystalline form of claim 26 or claim 27 wherein the crystalline form is crystalline Form C characterized by at least three x-ray powder diffraction peaks at 2Θ angles selected from 8.1°(±0.2°), 13.3°(±0.2°), 15.0°(±0.2°), 20.4°(±0.2°), 20.6°(±0.2°), 22.6°(±0.2°), and 25.3°(±0.2°).

29. The crystalline form of any one of claims 26-28, wherein the crystalline form is crystalline Form C characterized by at least four x-ray powder diffraction peaks at 2Θ angles selected from 8.1°(±0.2°), 13.3°(±0.2°), 15.0°(±0.2°), 20.4°(±0.2°), 20.6°(±0.2°), 22.6°(±0.2°), and 25.3°(±0.2°).

30. The crystalline form of any one of claims 26-29, wherein the crystalline form is crystalline Form C characterized by at least five x-ray powder diffraction peaks at 2Θ angles selected from 8.1°(±0.2°), 13.3°(±0.2°), 15.0°(±0.2°), 20.4°(±0.2°), 20.6°(±0.2°), 22.6°(±0.2°), and 25.3°(±0.2°).

31. The crystalline form of any one of claims 26-30, wherein the crystalline form is crystalline Form C characterized by at least six x-ray powder diffraction peaks at 2Θ angles selected from 8.1°(±0.2°), 13.3°(±0.2°), 15.0°(±0.2°), 20.4°(±0.2°), 20.6°(±0.2°), 22.6°(±0.2°), and 25.3°(±0.2°).

32. The crystalline form of any one of claims 26-31, wherein the crystalline form is crystalline Form C characterized by x-ray powder diffraction peaks at 2Θ angles selected from 8.1°(±0.2°), 13.3°(±0.2°), 15.0°(±0.2°), 20.4°(±0.2°), 20.6°(±0.2°), 22.6°(±0.2°), and 25.3°(±0.2°).

33. The crystalline form of any one of claims 26-32, wherein the crystalline form is crystalline Form C characterized by an x-ray powder diffraction pattern substantially the same as depicted in FIG. 7.

34. The crystalline form of claim 26, wherein the crystalline form is crystalline Form F characterized by at least two x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.20).

35. The crystalline form of claim 26 or claim 34 wherein the crystalline form is crystalline Form F characterized by at least three x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°).

36. The crystalline form of any one of claims 26 or 34-35, wherein the crystalline form is crystalline Form F characterized by at least four x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°).

37. The crystalline form of any one of claims 26 or 34-36, wherein the crystalline form is crystalline Form F characterized by at least five x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°).

38. The crystalline form of any one of claims 26 or 34-37, wherein the crystalline form is crystalline Form F characterized by at least six x-ray powder diffraction peaks at 2Θ angles selected from 4.8°(±0.2°), 9.6°(±0.2°), 11.3°(±0.2°), 14.5°(±0.2°), 18.2°(±0.2°), 19.3°(±0.2°), 20.2°(±0.2°), and 22.8°(±0.2°).

39. The crystalline form of any one of claims 26 or 34-38, wherein the crystalline form is crystalline Form F characterized by x-ray powder diffraction peaks at 2Θ angles selected from 4.8°(±0.20), 9.60(±0.20), 11.3°(±0.20), 14.5°(±0.20), 18.2°(±0.20), 19.3°(±0.20), 20.2°(±0.2°), and 22.8°(±0.2°).

40. The crystalline form of any one of claims 26 or 34-39, wherein the crystalline form is crystalline Form F characterized by an x-ray powder diffraction pattern substantially the same as depicted in FIG. 42.

41. The crystalline form of claim 26, wherein the crystalline form is crystalline Form G characterized by at least two x-ray powder diffraction peaks at 2Θ angles selected from 5.7°(±0.2°), 7.2°(±0.2°), 7.7°(±0.2°), 11.3°(±0.2°), 16.8°(±0.2°), 18.3°(±0.2°), and 20.1°(±0.2°).

42. The crystalline form of claim 26 or claim 41 wherein the crystalline form is crystalline Form G characterized by at least three x-ray powder diffraction peaks at 2Θ angles selected from 5.7°(±0.2°), 7.2°(±0.2°), 7.7°(±0.2°), 11.3°(±0.2°), 16.8°(±0.2°), 18.3°(±0.2°), and 20.1°(±0.2°).

43. The crystalline form of any one of claims 26 or 41-42, wherein the crystalline form is crystalline Form G characterized by at least four x-ray powder diffraction peaks at 2Θ angles selected from 5.7°(±0.2°), 7.2°(±0.2°), 7.7°(±0.2°), 11.3°(±0.2°), 16.8°(±0.2°), 18.3°(±0.2°), and 20.1°(±0.2°).

44. The crystalline form of any one of claims 26 or 41-43, wherein the crystalline form is crystalline Form G characterized by at least five x-ray powder diffraction peaks at 2Θ angles selected from 5.7°(±0.2°), 7.2°(±0.2°), 7.7°(±0.2°), 11.3°(±0.2°), 16.8°(±0.2°), 18.3°(±0.2°), and 20.1°(±0.2°).

45. The crystalline form of any one of claims 26 or 41-44, wherein the crystalline form is crystalline Form G characterized by at least six x-ray powder diffraction peaks at 2Θ angles selected from 5.7°(±0.2°), 7.2°(±0.2°), 7.7°(±0.2°), 11.3°(±0.2°), 16.8°(±0.2°), 18.3°(±0.2°), and 20.1°(±0.2°).

46. The crystalline form of any one of claims 26 or 41-44, wherein the crystalline form is crystalline Form G characterized by x-ray powder diffraction peaks at 2Θ angles selected from 5.70(±0.20), 7.20(±0.20), 7.70(±0.20), 11.3°(±0.20), 16.8°(±0.20), 18.3°(±0.20), and 20.1°(±0.20).

47. The crystalline form of any one of claims 26 or 41-46, wherein the crystalline form is crystalline Form G characterized by an x-ray powder diffraction pattern substantially the same as depicted in FIG. 44.

48. A crystalline form of a tartrate salt of Compound 1 having the structural formula:

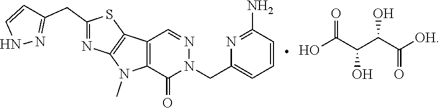 [SMILES: Cn1c2nc(Cc3cc[nH]n3)sc2c2cnn(Cc3cccc(N)n3)c(=O)c21 ; O=C(O)[C@@H](O)[C@H](O)C(=O)O]

49. The crystalline form of claim 48, wherein the crystalline form is crystalline Form I characterized by at least two x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.20).

50. The crystalline form of claim 48 or claim 49, wherein the crystalline form is crystalline Form I characterized by at least three x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°).

51. The crystalline form of any one of claims 48-50, wherein the crystalline form is crystalline Form I characterized by at least four x-ray powder diffraction peaks at 2Θ angles selected from 8.0°(±0.2°), 10.8°(±0.2°), 13.8(±0.2°), 15.0°(±0.2°), 16.0°(±0.2°), 16.7°(±0.2°), and 25.2°(±0.2°).

52. The crystalline form of any one of claims 48-51, wherein the crystalline form is crystalline Form I characterized by at least five x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.2°).

53. The crystalline form of any one of claims 48-52, wherein the crystalline form is crystalline Form I characterized by x-ray powder diffraction peaks at 2Θ angles selected from 8.0°(±0.2°), 10.8°(±0.2°), 13.8°(±0.2°), 15.0°(±0.2°), 16.0°(±0.2°), 16.7°(±0.2°), and 25.2°(±0.20).

54. The crystalline form of any one of claims 48-53, wherein the crystalline form is crystalline Form H characterized by an x-ray powder diffraction pattern substantially the same as depicted in FIG. 35.

55. A crystalline form of a hydrochloride salt of Compound 1 having the structural formula:

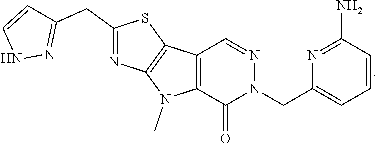 [SMILES: Cn1c2nc(Cc3cc[nH]n3)sc2c2cnn(Cc3cccc(N)n3)c(=O)c21]

56. The crystalline form of claim 55, wherein the crystalline form is crystalline Form D characterized by at least two x-ray powder diffraction peaks at 2Θ angles selected from 10.6°(±0.2°), 14.4°(±0.2°), 24.6°(±0.2°), 24.8°(±0.2°), 25.2°(±0.2°), and 27.0°(±0.2°).

57. The crystalline form of any one of claim 55-56, wherein the crystalline form is crystalline Form D characterized by at least three x-ray powder diffraction peaks at 2Θ angles selected from 10.6°(±0.2°), 14.4°(±0.2°), 24.6°(±0.2°), 24.8°(±0.2°), 25.2°(±0.2°), and 27.0°(±0.20).

58. The crystalline form of any one of claims 55-57, wherein the crystalline form is crystalline Form D characterized by at least four x-ray powder diffraction peaks at 2Θ angles selected from 10.6°(±0.2°), 14.4°(±0.2°), 24.6°(±0.2°), 24.8°(±0.2°), 25.2°(±0.2°), and 27.0°(±0.20).

59. The crystalline form of any one of claims 55-58, wherein the crystalline form is crystalline Form D characterized by at least five x-ray powder diffraction peaks at 2Θ angles selected from 10.6°(±0.2°), 14.4°(±0.2°), 24.6°(±0.2°), 24.8°(±0.2°), 25.2°(±0.2°), and 27.0°(±0.20).

60. The crystalline form of any one of claims 55-59, wherein the crystalline form is crystalline Form D characterized by x-ray powder diffraction peaks at 2Θ angles selected from 10.6°(±0.2°), 14.4°(±0.2°), 24.6°(±0.2°), 24.8°(±0.2°), 25.2°(±0.2°), and 27.0°(±0.20).

61. The crystalline form of any one of claims 55-60, wherein the crystalline form is crystalline Form D characterized by an x-ray powder diffraction pattern substantially the same as depicted in FIG. 10 or FIG. 27 or FIG. 31.

62. The crystalline form of claim 55, wherein the crystalline form is crystalline Form J characterized by at least two x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.20).

63. The crystalline form of claim 55 or claim 62, wherein the crystalline form is crystalline Form J characterized by at least three x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°).

64. The crystalline form of any one of claims 55 or 62-63, wherein the crystalline form is crystalline Form J characterized by at least four x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°).

65. The crystalline form of any one of claims 55 or 62-64 wherein the crystalline form is crystalline Form J characterized by at least five x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.2°).

66. The crystalline form of any one of claims 55 or 62-65, wherein the crystalline form is crystalline Form J characterized by x-ray powder diffraction peaks at 2Θ angles selected from 15.0°(±0.2°), 15.6°(±0.2°), 20.4(±0.2°), 20.6°(±0.2°), 21.1°(±0.2°), 24.1°(±0.2°), and 24.7°(±0.20).

67. The crystalline form of any one of claims 55 or 62-66, wherein the crystalline form is crystalline Form J characterized by an x-ray powder diffraction pattern substantially the same as depicted in FIG. 46.

68. The crystalline form of claim 55, wherein the crystalline form is crystalline Form K characterized by at least two x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°).

69. The crystalline form of claim 55 or claim 68, wherein the crystalline form is crystalline Form K characterized by at least three x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°).

70. The crystalline form of any one of claims 55 or 68-69, wherein the crystalline form is crystalline Form K characterized by at least four x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°).

71. The crystalline form of any one of claims 55 or 68-70 wherein the crystalline form is crystalline Form K characterized by at least five x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°).

72. The crystalline form of any one of claims 55 or 68-71, wherein the crystalline form is crystalline Form K characterized by x-ray powder diffraction peaks at 2Θ angles selected from 7.2°(±0.2°), 9.5°(±0.2°), 10.6(±0.2°), 14.0°(±0.2°), 17.4°(±0.2°), 26.0°(±0.2°), and 27.0°(±0.2°).

73. The crystalline form of any one of claims 55 or 68-72, wherein the crystalline form is crystalline Form K characterized by an x-ray powder diffraction pattern substantially the same as depicted in FIG. 48.

74. A crystalline free base of Compound 1 having the structural formula:

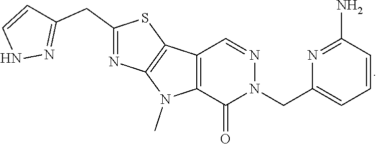 [SMILES: Cn1c2nc(Cc3cc[nH]n3)sc2c2cnn(Cc3cccc(N)n3)c(=O)c21]

75. The crystalline form of claim 74, wherein the crystalline form is crystalline Form E characterized by at least two x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°).

76. The crystalline form of claim 74 or 75, wherein the crystalline form is crystalline Form E characterized by at least three x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°).

77. The crystalline form of any one of claims 74-76, wherein the crystalline form is crystalline Form E characterized by at least four x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°).

78. The crystalline form of any one of claims 74-77, wherein the crystalline form is crystalline Form E characterized by at least five x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°).

79. The crystalline form of any one of claims 74-78, wherein the crystalline form is crystalline Form E characterized by at least six x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°).

80. The crystalline form of any one of claims 74-79, wherein the crystalline form is crystalline Form E characterized by x-ray powder diffraction peaks at 2Θ angles selected from 11.6°(±0.2°), 16.0°(±0.2°), 16.8°(±0.2°), 20.6°(±0.2°), 23.8°(±0.2°), 26.0°(±0.2°), and 27.4°(±0.2°).

81. The crystalline form of any one of claims 74-80 wherein the crystalline form is crystalline Form E characterized by an x-ray powder diffraction pattern substantially the same as depicted in FIG. 12.

82. A method for making a solid dispersion comprising:
(a) dissolving a crystalline form of Compound 1 of any one of claims 1 to 81 and a polymer in a solvent to form a feed solution; and
(b) removing the solvent to form the solid dispersion.

83. The solid dispersion of claim 82, wherein the crystalline form of Compound 1 used in the preparation is the crystalline hemi-hydrate hemi-sulfate salt, the crystalline free base, the crystalline hydrochloride salt, or the crystalline phosphate salt as defined in any one of claims 1-25 and 55-81.

84. The method of either claim 82 or 83 wherein the solid dispersion is a spray dried dispersion.

85. The method of any one of claims 82-84, wherein the solid dispersion comprises a polymer selected from hypromellose acetate succinate based amorphous solid, medium grad (HPMCAS-MG), and polyvinylpyrrolidone/vinyl acetate copolymer (PVPVA).

86. The method of any one of claims 82-85, wherein the solvent is methanol, ethanol, n-propanol, isopropanol, butanol, acetone, methyl ethyl ketone, methyl isobutyl ketone, ethyl acetate, propyl acetate, tetrahydrofuran (THF), acetonitrile (ACN), dichloromethane, toluene, or a mixture thereof.

87. The method of any one of claims 82-86, wherein the solvent is a methanol/dichloromethane mixture.

88. The method of any one of claims 82-87, wherein the solid dispersion is formulated as a tablet.

89. The method of claim 87 or 88, wherein the tablet further comprises hypromellose acetate succinate based amorphous solid, medium grad, polyvinylpyrrolidone, tocofersolan, and simethicone.

90. A pharmaceutical composition comprising a crystalline form of Compound 1 according to any one of claims 1-81 and a pharmaceutically acceptable excipient.

91. A pharmaceutical composition comprising amorphous Compound 1 and a pharmaceutically acceptable excipient.

92. A method for reducing 2,3-diphosphoglycerate levels in blood of a subject in need thereof comprising contacting the blood of the subject with an effective amount of (1) a crystalline form of Compound 1 according to any one of claims 1-81; or (2) the pharmaceutical composition according to claim 90 or 91.

93. A method for treating anemia in a subject in need thereof comprising administering to the subject (1) a crystalline form of Compound 1 according to any one of claims 1-81; or (2) the pharmaceutical composition according to claim 90 or 91.

94. The method of claim 93, wherein the anemia is hemolytic anemia associated with acquired Pyruvate Kinase Deficiency (PKD).

95. The method of claim 93, wherein the anemia is associated with very low risk MDS, low risk MDS, lower risk MDS and/or intermediate risk MDS (myelodysplastic syndrome).

96. A method for treating hemolytic anemia in a subject in need thereof comprising administering to the subject (1) a crystalline form of Compound 1 according to any one of claims 1-81; or (2) the pharmaceutical composition according to claim 90 or 91.

97. The method of claim 96, wherein the hemolytic anemia is hereditary or congenital hemolytic anemia, acquired hemolytic anemia, or anemia associated with very low risk MDS, low risk MDS, lower risk MDS and/or intermediate risk MDS.

98. The method of claim 95 or 96 wherein the subject is administered 2, 5, 10, 15, 20, 25, or 30 mg of Compound 1 per day.

99. A method for treating sickle cell disease in a subject in need thereof comprising administering to the subject (1) a crystalline form of Compound 1 according to any one of claims 1-81; or (2) the pharmaceutical composition according to claim 90 or 91.

100. A method of increasing the amount of hemoglobin in a subject in need thereof comprising administering to the subject (1) a crystalline form of Compound 1 according to any one of claims 1-81; or (2) the pharmaceutical composition according to claim 90 or 91.

101. A method of forming a crystalline hemi-sulfate salt of any one of claims 1-17, the method comprising reacting a Compound 1: 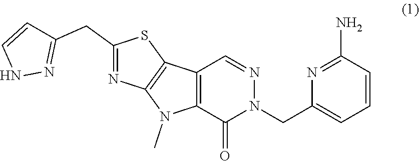 [SMILES: Cn1c2nc(Cc3cc[nH]n3)sc2c2cnn(Cc3cccc(N)n3)c(=O)c21]
with H₂SO₄ in an alcoholic solution.

102. The method of claim 101, wherein the alcoholic solution further comprises water.

103. The method of claim 102, wherein the ratio of alcohol to water is about 8:2 v/v.

104. The method of any one of claims 101-103, wherein the alcohol is methanol, ethanol, n-propanol, isopropyl alcohol, n-butanol, or isobutanol, or any combination thereof.

105. The method of any one of claims 101-104, wherein the alcohol is n-propanol.

106. A method of forming a crystalline Phosphate salt of any one of claims 18-25, the method comprising reacting a Compound 1: 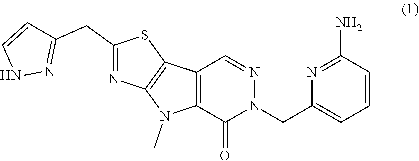 [SMILES: Cn1c2nc(Cc3cc[nH]n3)sc2c2cnn(Cc3cccc(N)n3)c(=O)c21]
with H₃PO₄ in a polar aprotic solution.

107. The method of claim 106, wherein the polar aprotic solvent is acetone, acetonitrile, dichloromethane, dimethylformamide, dimethyl sulfoxide, pyridine, or tetrahydrofuran, or any combination thereof.

108. The method of claim 106 or 107, wherein the polar aprotic solvent is dimethylsulfoxide.

109. The method of any one of claims 106-108, wherein the method further comprises a second step of adding an antisolvent.

110. The method of claim 109, wherein the antisolvent is an alcoholic solution.

111. The method of claim 110, wherein the alcoholic solution further comprises water.

112. The method of claim 111, wherein the ratio of alcohol to water is about 8:2 v/v.

113. The method of any one of claims 110-112, wherein the alcohol is methanol, ethanol, n-propanol, isopropyl alcohol, n-butanol, or isobutanol, or any combination thereof.

114. The method of any one of claims 110-113, wherein the alcohol is ethanol.

115. A method of forming a crystalline DL-tartrate salt of any one of claims 26-47, the method comprising reacting a Compound 1: 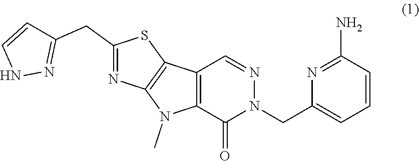 [SMILES: Cn1c2nc(Cc3cc[nH]n3)sc2c2cnn(Cc3cccc(N)n3)c(=O)c21]
with a solution comprising DL-tartaric acid and at least one polar aprotic solvent.

116. The method of claim 115, wherein the polar aprotic solvent is selected from acetone, acetonitrile, dichloromethane, dimethylformamide, dimethyl sulfoxide, pyridine, or tetrahydrofuran, or any combination thereof.

117. The method of claim 115 or claim 116, wherein the polar aprotic solvent is tetrahydrofuran.

118. The method of any one of claims 115-117, wherein the solvent further comprises water.

119. The method of claim 118, wherein the ratio of solvent to water is about 99:1, about 95:5, about 90:10, about 80:20, or about 60:40 v/v.

120. The method of claim 119, wherein the ratio of solvent to water is about 95:5 v/v.

121. A method of forming a crystalline L-tartrate salt of any one of claims 48-54, the method comprising reacting a Compound 1: 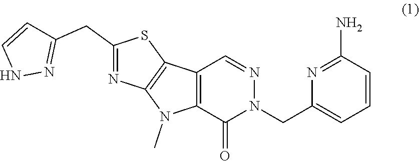 [SMILES: Cn1c2nc(Cc3cc[nH]n3)sc2c2cnn(Cc3cccc(N)n3)c(=O)c21]
with a solution comprising L-tartaric acid in a solution.

122. The method of claim 121, wherein solution comprises L-tartaric acid, water, and an alcohol.

123. The method of claim 122, wherein the solution comprises a ratio of alcohol to water of about 99:1, about 95:5, about 90:10, about 80:20, or about 60:40 v/v.

124. The method of claim 122 and 123, wherein the solution comprises a ratio of alcohol to water of about 95:5 v/v.

125. The method of any one of claims 122-124, wherein the alcohol is selected from methanol, ethanol, n-propanol, isopropyl alcohol, n-butanol, or isobutanol, propylene glycol, ethylene glycol, t-amyl alcohol, diethylene glycol, n-pentanol, benzyl alcohol, or cyclohexanol or any combination thereof.

126. The method of claim 125, wherein the alcohol is ethanol.

127. A method of forming a crystalline hydrochloride salt of any one of claims 55-73, the method comprising reacting a Compound 1: 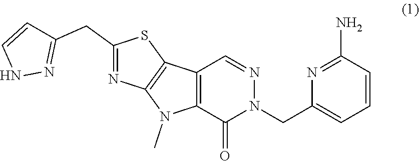 [SMILES: Cn1c2nc(Cc3cc[nH]n3)sc2c2cnn(Cc3cccc(N)n3)c(=O)c21]
with a solution comprising hydrochloric acid and at least one polar aprotic solvent.

128. The method of claim 127, wherein the polar aprotic solvent is selected from acetone, acetonitrile, dichloromethane, dimethylformamide, dimethyl sulfoxide, pyridine, or tetrahydrofuran, or any combination thereof.

129. The method of claim 127 or claim 128, wherein the polar aprotic solvent is tetrahydrofuran.

130. A method for treating anemia associated with acquired pyruvate kinase deficiency in a subject with very low risk MDS, low risk MDS, lower risk MDS, and/or intermediate risk MDS comprising administering to the subject (1) a crystalline form of Compound 1 according to any one of claims 1-81; or (2) the pharmaceutical composition according to claim 90 or 91.

131. The method of claim 130 wherein the anemia is hemolytic anemia.

132. The method of claim 130 or claim 131 wherein the subject is administered 2, 5, 10, 15, 20, 25, or 30 mg of Compound 1 per day.

133. The method of claim 99 wherein the subject is administered 2, 5, 10, 15, 20, 25, or 30 mg of Compound 1 per day.
